(19) United States
(12) Patent Application Publication
(10) Pub. No.: US 2025/0295715 A1
(43) Pub. Date: Sep. 25, 2025

(54) POSTBIOTIC-BASED COMPOSITION FOR USE IN THE TREATMENT OF SKIN DISEASES

(71) Applicant: POSTBIOTICA S.R.L., Milan (IT)

(72) Inventors: Maria RESCIGNO, Milan (IT); Giuseppe PENNA, Milan (IT)

(73) Assignee: POSTBIOTICA S.R.L., Milan (IT)

(21) Appl. No.: 19/213,533

(22) Filed: May 20, 2025

Related U.S. Application Data
(63) Continuation of application No. 16/965,469, filed on Jul. 28, 2020, now Pat. No. 12,324,820.

(30) Foreign Application Priority Data
Feb. 2, 2018 (IT) 102018000002369

(51) Int. Cl. A61K 35/747; A61K 8/99; A61K 35/00; A61P 17/06; A61P 17/10; A61P 17/12

(52) U.S. Cl. CPC A61K 35/747; A61K 8/99; A61P 17/06; A61P 17/10; A61P 17/12; A61K 2035/115

(57) ABSTRACT

The present invention refers to a composition including fermented supernatant of Lactobacillus casei or paracasei species. The species is characterized by having in its DNA genome at least one DNA sequence essentially identical to one of the sequences selected from the group consisting of: SEQ ID NO: 1 to 5, and proper diluents and/or carriers and/or excipients. The composition can be used in the treatment and/or prevention of skin and/or dermatological diseases related to inflammation, allergic reactions, contact hypersensitivity reactions and intolerance, excessive sebaceous secretion, exfoliation or microbial dysbiosis, preferably of the epidermis.

### CROSS-REFERENCE TO RELATED APPLICATIONS

This application is a Continuation of U.S. patent application Ser. No. 16/965,469, filed Jul. 28, 2020, which is 371 of PCT/EP2019/052665, filed Feb. 4, 2019, which claims the benefit of Italian Patent Application No. 102018000002369, filed Feb. 2, 2018, the entire content of each of which is incorporated by reference herein.

### REFERENCE TO A SEQUENCE LISTING SUBMITTED ELECTRONICALLY VIA PATENT CENTER

This application contains references to amino acid sequences and/or nucleic acid sequences which have been submitted concurrently herewith as the sequence listing text file entitled: 128-1227-C SEQLIST.xml; size: 81,562 bytes; and date of creation: May 13, 2025. The aforementioned sequence listing is hereby incorporated by reference in its entirety pursuant to 37 C.F.R. § 1.52(e)(5).

### FIELD OF INVENTION

The invention refers to a fermented product obtained from Lactobacillus casei or paracasei species, able to significantly reduce, in a dose dependent manner, IL-6 and IL-8 cytokines, chemotactic factor CCL2/MCP1 and long isoform “lfTSLP” (inflammatory) levels and increase the level of short isoform “sfTSLP” (homeostatic), in eukaryotic cells exposed to inflammatory stimuli, in particular epidermal cells, more in particular human keratinocytes. Said fermented product finds application in a variety of dermatological and cosmetic applications.

### BACKGROUND OF THE INVENTION

The skin is considered the organ with the largest surface of our body. It covers a protective, sensorial, secretory, thermoregulatory, absorption, permeability and aesthetic action. In utero, the skin is considered sterile and only after birth, the environmental microbial communities begin colonization of the stratum corneum, and evolving into a complex microbial ecosystem, which is actively involved in the formation and in the maintenance of cutaneous homeostasis and of a correct inflammatory response (Dominguez-Bello et al., 2010) (Capone et al., 2011). The complexes of microbes present on the skin surface, whether resident or transient, are called skin microbiota (Cho and Blaser, 2012). The interaction between host and microbiota is fundamental for the development and maintenance of the symbiotic status. The bioactive molecules derived from the bacteria metabolism during the fermentation process, called postbiotics, constitute the molecular signals underlying this interaction. In some pathological conditions the dysbiosis of the cutaneous microbiota, can contribute to immunological homeostasis breaking thus amplifying the symptoms of the disease (Kong et al., 2012) (Naik et al., 2012). Modifications of the cutaneous microbiota have been associated with the rising of allergies and chronic inflammatory epidermal diseases (Ehlers et al., 2010). The immune response of the skin, in particular the production of cytokines by epidermal keratinocytes, is influenced by cutaneous microbiota, as shown by the significant increase in “germ free” mice, devoid of microbiota, of the cytokine Thymic Stromal Lymphopoietin (TSLP) (Yockey et al., 2013). The TSLP protein is an immunological mediator, involved both in multiple immunological diseases (Ziegler, 2012) (Siracusa et al., 2011) and in the maintenance of immunological tolerance (Watanabe et al., 2005) (Hanabuchi et al., 2010). The identification of two distinct forms of TSLP, the long (lfTSLP) and short (sfTSLP) form and their functional characterization have shed light on the opposite biological functions of the molecules. The lfTSLP exerts a pathogenic role and is induced by inflammatory stimuli while the sfTSLP is released constitutively and exerts an anti-inflammatory and homeostatic role (Fornasa et al., 2015). The imbalance of the 2 isoforms, favoring the inflammatory form lfTSLP has been identified in skin diseases such as atopic dermatitis (Fornasa et al., 2015). The release of inflammatory mediators, such as IL-6, IL8, CCL2/MCP1 and inflammatory TSLP (lfTSLP), by skin keratinocytes, is the basis for multiple skin diseases (Jang et al., 2013) (Wilson et al., 2013) (Schaper et al., 2016) (Volpe et al., 2014). In particular, human epidermal keratinocytes express the inflammatory form of TSLP (lfTSLP), only in response to inflammatory stimuli such as polyinosinic:polycytidylic acid (poly I:C) or invasive bacteria such as Salmonella typhimurium, while constitutively release the homeostatic form of TSLP (sfTSLP), which is reduced by pathogenic bacteria (Salmonella typhimurium) and increased by anti-inflammatory agents such as vitamin D3 (Fornasa et al., 2015). Furthermore, the sfTSLP form exerts an antibacterial activity, through the modulation of the microbiota, favoring the skin protective action (Bjerkan et al., 2015).

### Probiotics and Postbiotics

Probiotics are defined as live microorganisms that exert beneficial effects on the host when administered in adequate amounts. Probiotics are in general isolated from fecal samples of healthy individuals, mostly from breast-fed infants. The microbiota may belong to either symbiotic or pathobiont classes of microorganisms and may have divergent immunomodulatory properties. Different strains, even among the same species, may have opposing effects, as has been shown in a number of studies (Kaci et al., 2011; Van Hemert et al., 2010). Furthermore, recent data suggest that certain beneficial effects observed after the administration of probiotics may be mediated by molecules or factors produced and secreted by the bacteria into the gut lumen, henceforth herein called postbiotics (or “fermented product” or “fermented supernatant”). In the context of the present invention, for postbiotic is intended any factor resulting from the metabolic activity of a probiotic or any released molecule capable of conferring beneficial effects to the host in a direct or indirect way.

### Lactobacillus paracasei Supernatant

The putative therapeutic use of strains of Lactobacillus paracasei strain CNCM I-1390 (Budapest Treaty deposit), redeposited by IEO—Istituto Europeo di Oncologia S.r.l., Via dei Filodrammatici 10, 20121 Milano, on Jul. 26, 2017, according to Budapest Treaty, with CNCM (Collection Nationale de Cultures de Microorganismes, Institut Pasteur, 25,28 rue du Docteur Roux 75724 Paris CEDEX 15, FR) with the no. I-5220 (hereinafter also named as B21060), in particular of the fermentation supernatant thereof as an anti-inflammatory in intestinal diseases, is described in WO 2011/009848 A2.

It is still felt the need of a postbiotic, able to prevent or treat dermatological diseases.

### DESCRIPTION OF THE INVENTION

Lactobacillus casei and Lactobacillus paracasei have been reclassified as Lacticaseibacillus casei and Lacticaseibacillus paracasei, respectively. See “A taxonomic note on the genus Lactobacillus” in Zheng et al., Int. J. Syst. Evol. Microbiol. 2020; 70:2782-2858 (DOI 10.1099/ijsem.0.004107).

In this specification and claims:
Lacticaseibacillus casei and Lactobacillus casei will also be referred to L. casei;
Lacticaseibacillus paracasei and Lactobacillus paracasei will also be referred to L. paracasei;
Lacticaseibacillus casei, Lactobacillus casei, and L. casei are used interchangeably; and
Lacticaseibacillus paracasei, Lactobacillus paracasei, and L. casei are used interchangeably.

The inventors have found that the metabolic products or the supernatant (herein both and/or together defined as “fermented product”) by Lactobacillus paracasei strain CNCM I-5220, of Institute Pasteur collection, is able to modulate keratinocyte activation, favouring the inflammatory response, the epidermal and cutaneous microbiota homeostasis. The present invention relates to the use of a formulation containing the postbiotic generated by bacterial strain Lactobacillus paracasei CNCM I-5220 fermentation, in pathological epidermal manifestation such as psoriasis, atopic and contact dermatitis, eczema, rosacea, acne, surgical scars and sunburn, itching, cold sores and dry skin (senile xerosis, asteatosis).

It is therefore object of the present invention a composition comprising a fermented supernatant (or fractions thereof) of Lactobacillus casei or paracasei species, said species being characterized by comprising in its DNA genome at least one DNA sequence essentially identical to one of the sequences selected from the group consisting of: SEQ ID No: 1 to 5, and proper diluents and/or carriers and/or excipients for use in the treatment and/or prevention of skin and/or dermatological diseases related to inflammation, allergic reactions, contact hypersensitivity reactions and intolerance, excessive sebaceous secretion, exfoliation or microbial dysbiosis (modification of the microbiota composition), preferably of the epidermis.

Said pathology is preferably selected from the group consisting of: psoriasis, contact and atopic dermatitis, eczema, rosacea, acne, surgical scars and sunburn, itching, cold sores, dry skin (e.g. xerosis senile, asteatosis), greasy or itchy dandruff, trichodynia, seborrheic dermatitis.

Said fermented supernatant is preferably obtainable by a method characterized by the fermentation of said Lactobacillus casei or paracasei species in a minimum solution additioned with a lactate salt, preferably sodium lactate, calcium lactate, potassium lactate.

Preferably, said minimum solution is a solution which does not contain carbon and/or nitrogen sources or micromolar concentration of minerals (e.g. iron, sulfur etc.), more preferably it is saline, phosphate buffer, H2O, a minimum isotonic solution or a hypotonic solution.

Preferably, said method comprises two fermentations of said Lactobacillus casei or paracasei species into a minimum solution, at least one of which is carried out into a minimum solution additioned with a lactate salt, preferably sodium lactate, calcium lactate, potassium lactate.

In a preferred embodiment of the invention said second fermentation is carried out at a temperature of 0° C.-10° C., preferably of 4° C.

Preferably, said fermented supernatant is a dry powder, e.g. obtained by lyophilization, freeze-drying, granulation, spray drying.

Preferably, the fermented supernatant comprises:
a) after a first fermentation into a minimum solution:
oleic acid and/or decanoic acid and/or benzopropanoic acid and/or citric acid, preferably in the following concentration:
oleic acid: 3-9 mg/L, preferably 4-8 mg/L, more preferably about 7.98 mg/L; and/or
decanoic acid: 1-3 mg/L, preferably about 1.30 mg/L; and/or
benzopropanoic acid: 1-4 mg/L, preferably 2-3 mg/L; more preferably about 2.69 mg/L and/or
citric acid: 10-15 mg/L, preferably 12-14 mg/L; more preferably about 13.05 mg/L. and/or
b) after a second fermentation into a minimum solution additioned with a lactate salt, preferably sodium lactate:
7 hexadecenoic acid and/or tridecanoic acid and/or vaccenic acid and/or oleic acid, preferably in the following concentration:
7 hexadecenoic acid: 2-6 mg/L; 5-6 mg/L; about 5.12 mg/L; and/or
tridecanoic acid: 2-6 mg/L; 5-6 mg/L; about 5.42 mg/L; and/or
vaccenic acid: 10-15 mg/L; preferably 12-14 mg/L; more preferably about 12.23 mg/L and/or
oleic acid: 3-5 mg/L, preferably 3-4 mg/L, more preferably about 3.21 mg/L.

More preferably, the fermented supernatant comprises after a first fermentation into a minimum solution:
oleic acid, decanoic acid, benzopropanoic acid, citric acid, in the following concentration:

| | mg/L |
|---|---|
| oleic acid | 7.98 |
| decanoic acid | 1.30 |
| benzopropanoic acid | 2.69 |
| citric acid | 13.05 |

More preferably, the fermented supernatant comprises after a second fermentation into a minimum solution additioned with a lactate salt, preferably sodium lactate: 7 hexadecenoic acid and tridecanoic acid and vaccenic acid and oleic acid, in the following concentration:

| | mg/L |
|---|---|
| 7 hexadecenoic acid | 5.12 |
| tridecanoic acid | 5.42 |
| vaccenic acid | 12.23 |
| oleic acid | 3.21 |

Preferably, the fermented supernatants, when analyzed by MALDI TOF mass spectrometry, are characterized by the peptide signal profile of Table 2A.

Preferably, the fermented supernatant after two fermentations of said Lactobacillus casei or paracasei species into a minimum solution, at least one of which is carried out into a minimum solution additioned with a lactate salt (such as sodium lactate), when analyzed by MALDI TOF/TOF mass spectrometry, is characterized by the peptide signal profile of Table 2B.

Preferably, the fermented supernatant after two fermentations of said Lactobacillus casei or paracasei species into a minimum solution, at least one of which is carried out into a minimum solution additioned with sodium lactate, is characterized by comprising at least one of the peptides of SEQ ID NO: 19-41.

Said fermented supernatant is preferably obtainable by a method comprising the following steps:
a) growing an inoculum of Lactobacillus strain as defined above in a suitable culture medium, at a temperature ranging from 4 to 40° C., preferably of 37° C., to obtain a biomass and allowing fermentation of said biomass into a minimum solution to proceed for 12 to 36 hours, preferably for about 24 hours, to get a fermented biomass;
b) centrifuging said fermented biomass to get a pellet of fermented biomass and a first fermented product;
c) incubating said pellet of fermented biomass into a minimum solution for 12 to 36 hours, preferably for about 24 hours, at a temperature ranging from 4 to 40° C., preferably ranging from 4 to 20° C., more preferably of 4° C., to obtain a further fermented biomass;
d) separating said further fermented biomass from a second fermented product by centrifugation.

Preferably, one of the minimum solutions from step a) and c) comprises a lactate salt, such as sodium lactate, preferably the minimum solution of step c) comprises a lactate salt, such as sodium lactate.

First and/or second fermented product obtainable from step b) or d) respectively are used in the invention and collectively will be defined as “fermented product”. They may be used as active ingredients for the composition and formulation accordingly to the invention, either individually or combined. In a preferred embodiment of the invention, the fermented supernatant is the product obtainable from step d), more preferably obtainable by a method wherein only the minimum solution of step c) comprises a lactate salt, such as sodium lactate. The minimal solution of step a) and c) is for example a saline solution, phosphate buffer, H₂O, etc.

In the present invention the above species is preferably characterized by comprising in its DNA genome DNA sequences essentially identical to SEQ ID No 1-5.

Preferably, the Lactobacillus species is Lactobacillus paracasei, more preferably a strain characterized by comprising in its DNA genome at least one DNA sequence essentially identical to sequences SEQ ID No 6-18. Preferably said strain comprises in its DNA genome DNA sequences essentially identical to SEQ ID No 6-18.

Preferably, the Lactobacillus paracasei is the strain deposited according to Budapest Treaty with no. CNCM I-5220.

The strain B21060 (or CNCM I-5220) was deposited under the Budapest Treaty at Collection Nationale de Cultures de Microorganismes (CNCM), with number CNCM I-5220 on 26 Jul. 2017 (deposit information:

Microorganism Deposit Accession No.: CNCM I-5220;

Depositary Institution name: Collection nationale de cultures de microorganismes (CNCM));

Depositary Institution address: Institut Pasteur, 25 Rue du docteur Roux, 75724 Parise Cedex 15, France;

Deposit Date: 26 Jul. 2017

Name and Address of Depositor: IEO—Istituto Europeo di Oncologia S.r.l., Via Filodrammatici 10, 20121 Milano, Italy).

In a preferred embodiment, the composition further comprises at least one adjuvant and/or other therapeutic agents.

Preferably, the composition according to the invention is in a formulation for topical administration, preferably selected from the group consisting of: cream, emulsion, dispersion, gel, ointment, lotion, serum.

In the composition as above defined, the fermented product is preferably present at 0.1-1% w/w. A further object of the invention is the cosmetic use of the composition as defined above, preferably to prevent and/or reduce the signs of skin aging, skin imperfections, hyperhidrosis and for hair care. The composition for use according to the invention is preferably a pharmaceutical composition. The composition according to the present invention may further comprises at least one adjuvant and/or other therapeutic agents.

Preferably, the above defined species are characterized by at least one of the gene selected from the group consisting of SEQ ID NO. 1-5 and/or by at least one of the gene selected from the group consisting of SEQ ID NO. 6-8 and/or by at least one of the genome DNA sequences selected from the group consisting of SEQ ID NO:9-18. Preferably, the above defined species are characterized by the genes having the sequences essentially identical to SEQ ID NO. 1-5. Preferably, the above defined species are characterized by the genes having the sequences essentially identical to SEQ ID NO. 6-8. Preferably, the above defined species are characterized by genome DNA sequences essentially identical to SEQ ID NO:9-18. In the context of the present invention, for postbiotic is intended the supernatant or any factor resulting from the metabolic activity of a probiotic or any released molecule capable of conferring beneficial effects to the host in a direct or indirect way. Therefore, the term “postbiotic”, “fermented product” or “fermented supernatant” in the context of the present invention may comprise also any factor resulting from the metabolic activity of a probiotic or any released molecule capable of conferring beneficial effects to the host in a direct or indirect way. In the context of the present invention the term “fermented product” and “fermented supernatant” are exchangeable. In the context of the present invention the fermented supernatant also comprises any factor resulting from the metabolic activity of a probiotic or any released molecule capable of conferring beneficial effects to the host in a direct or indirect way. The preferred supernatant of the invention is a postbiotic product obtained by the fermentation of L. paracasei CNCM I-5220 and does not contain food and live bacteria. Said preferred embodiment is preferably lyophilized. In the context of the present invention “the solution supernatant” or the “fermented supernatant” may be also defined as “fermented product” and may also include fractions thereof and/or metabolic components thereof. The fermented product according to the invention is preferably obtained at a non-canonical fermentation temperature. The fermentation product used in the present invention may be obtained through the fermentation of different substrates such as sodium lactate or prebiotic ingredient (GOS,FOS, etc), plant extracts (e.g. Aloe, chamomile, mallow, Echinacea). The fermented product or composition according to invention may be lyophilized according to any method known to the skilled in the art. The fermented supernatant may be a dry powder, e.g. obtained by lyophilization, freeze-drying, granulation, spray drying. The pharmaceutical composition according to the invention is formulated to be administered to a subject in a therapeutically effective amount, depending on the type of subject, severity of the disease and route of administration. Typically, the therapeutically effective amount of the fermented product is about 0.01-10 mg/day, preferably 1 mg/day. The administration is e.g. carried out with 1-2 administrations/die of 0.5-5 g of fermented product or composition as defined above, at the concentration of 0.01-5% by weight of the final composition. In a further preferred embodiment, the composition comprises at least an additional ingredient, preferably selected from vitamin C, vitamin C derivatives, vitamin E, vitamin E derivatives, tocopherols, vitamin A, vitamin A derivatives, retinal, retinoic acid, vitamin D3, Vitamin K, Vitamin B1, B3, B5, B6, B12 and chemical elements such as zinc or essential amino acids, prebiotic ingredients and plant extracts (echinacea, mallow, chamomile, aloe, etc.) In the context of the present invention, the term fermented supernatant also comprises fractions thereof. In the context of the present invention, when referring to specific DNA sequences, it is intended that it is comprised within the invention also RNA molecules identical to said polynucleotides, except for the fact that the RNA sequence contains uracil instead of thymine and the backbone of the RNA molecule contains ribose instead of deoxyribose, RNA sequence complementary to the sequences herein disclosed, functional fragments, mutants and derivatives thereof, proteins encoded therefrom, functional fragments, mutants and derivatives thereof. The term “complementary” sequence refers to a polynucleotide, which is non-identical to the sequence, but either has a base sequence to the first sequence complementary or encodes the same amino acid sequence as the first sequence. A complementary sequence may include DNA and RNA polynucleotides. The term “functional” or “functional” may be understood as capable of maintaining the same activity. “Fragments” are preferably long at least 10 aa., 20 aa., 30 aa., 40 a., 50 aa., 60 aa., 70 aa., 80 aa., 90 aa., 100 aa., 150 aa., 200 aa., 300 aa., 400 aa., 500 aa., 600 aa., 700 aa., 800 aa., 900 aa., 1000 aa., 1200 aa., 1400 aa., 1600 aa., 1800 aa. or 2000 aa. “Derivatives” may be recombinant or synthetic. The term “derivative” as used herein in relation to a protein means a chemically modified protein or an analogue thereof, wherein at least one substituent is not present in the unmodified protein or an analogue thereof, i.e. a protein that has been covalently modified. Typical modifications are amides, carbohydrates, alkyl groups, acyl groups, esters and the like. As used herein, the term “derivatives” also refers to longer or shorter polynucleotides/proteins and/or having e.g. a percentage of identity of at least 85%, 86%, 87%, 88%, 89%, 90%, 91%, 92%, 93%, 94%, 95%, 96%, 97%, 98%, more preferably of at least 99% with the sequences herein disclosed. In the present invention “at least 70% identity” means that the identity may be at least 70%, or 75%, or 80%, or 85% or 90% or 95% or 100% sequence identity to referred sequences. This applies to all the mentioned % of identity. Preferably, the % of identity relates to the full length of the referred sequence. Sequence alignment to determine percental sequence identity may be determined according to any method known to the skilled in the art, for example using public domain software such as BLAST, BLAST-2, ALIGN o Megalign (DNASTAR) software. In the context of the present invention a carrier may be any vehicle or composition involved in delivery of the fermented product into the subject or that facilitate the storage of the composition. The present invention finds application in the treatment of humans and in veterinary. The derivative of the invention also includes “functional mutants” of the polypeptides, which are polypeptides that may be generated by mutating one or more amino acids in their sequences and that maintain their activity. Indeed, the polypeptide of the invention, if required, can be modified in vitro and/or in vivo, for example by glycosylation, myristoylation, amidation, carboxylation or phosphorylation, and may be obtained, for example, by synthetic or recombinant techniques known in the art. In the present invention “functional” is intended for example as “maintaining their activity” e.g. immunomodulatory activity or anti-inflammatory activity. Also within the scope of the subject invention are polynucleotides which have the same nucleotide sequences of a polynucleotide exemplified herein except for nucleotide substitutions, additions, or deletions within the sequence of the polynucleotide, as long as these variant polynucleotides retain substantially the same relevant functional activity as the polynucleotides specifically exemplified herein (e.g., they encode a protein having the same amino acid sequence or the same functional activity as encoded by the exemplified polynucleotide). Thus, the polynucleotides disclosed herein should be understood to include mutants, derivatives, variants and fragments, as discussed above, of the specifically exemplified sequences. The subject invention also contemplates those polynucleotide molecules having sequences which are sufficiently homologous with the polynucleotide sequences of the invention so as to permit hybridization with that sequence under standard stringent conditions and standard methods (Maniatis, T. et al, 1982). Polynucleotides described herein can also be defined in terms of more particular identity and/or similarity ranges with those exemplified herein. The sequence identity will typically be greater than 60%, preferably greater than 75%, more preferably greater than 80%, even more preferably greater than 90%, and can be greater than 95%. The identity and/or similarity of a sequence can be of 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% or greater as compared to a sequence exemplified herein. According to the present invention, the pH of the fermentation medium is adjusted to be within the range of 5-7.5 prior to the fermentation. It is preferable that the pH be adjusted within the above-mentioned range, for example, by diluting with distilled water, etc., without using a pH adjustor after suitably processing the vegetative raw material, or by suitably adjusting the kind or amount of the vegetative raw material. If it is necessary to use a pH adjustor, one which is generally used for foods may be added as long as it does not deteriorate the effect of the present invention, and the kind thereof is not particularly limited. Examples of a preferable acid include citric acid, potassium sorbate, and examples of a preferable base include potassium carbonate. If the pH adjustor used is in a form of crystals, it is preferable to use an aqueous solution thereof. It is preferable that Lactobacillus casei or paracasei as defined above is used for the fermentation of medium after being precultivated. Using conventional methods the precultivation may carry out. Fermentation of the medium using Lactobacillus casei or paracasei may be carried out by using conventional methods. For example, the above-mentioned precultivated product may be inoculated so as to cultivate said Lactobacillus casei or paracasei. The amount of inoculation is preferably in the range of 0.1 to 10% by volume, the temperature during the cultivation is in the range of 4 to 40° C., preferably of 37° C. and the time period for the cultivation is preferably 16-30 hours. According to the present invention, the pH of the fermented product at the end of the fermentation is 5.5 or greater and less than 7.0. The composition according to the invention also comprises a fermented product of Lactobacillus casei or paracasei having the same bacteriological properties as those of the CNCM I-5220 strain. A further object of the invention is a fermented supernatant of Lactobacillus casei or paracasei species, as defined above, for use in the treatment and/or prevention of skin and/or dermatological diseases related to inflammation, allergic reactions, contact hypersensitivity reactions and intolerance, excessive sebaceous secretion, exfoliation or microbial dysbiosis (modification of the microbiota composition), preferably of the epidermis. The fermentation product of the present invention is preferably obtained by fermentation using a CNCM I-5220 strain, or a mutant strain of the CNCM I-5220 strain having the same ability, or a strain isolated from Lactobacillus casei or paracasei having the same bacteriological properties as those of the CNCM I-5220 strain. In particular, the second fermentation is preferably performed under anaerobic conditions at a temperature of 4 to 40° C., preferably 4 to 37° C., in particular 4° C. The fermentation is desired to be performed at a pH of 4 to 8.0, preferably 5.0 to 7.0, particularly preferably about 6.5. It should be noted that the above-mentioned fermentation product is desirably contained at a concentration of 1 to 20% or more in terms of dry weight. The fermented product as described above is therefore used as an active ingredient in the preparation of compositions for dermatological and/or cosmetic use. These compositions can be used, for example, to prevent or reduce the phenomenon of skin aging, in particular wrinkles, and/or to improve the appearance of the skin or scalp (in particular reduction of sebaceous secretion, itching due to dandruff and rebalancing of the hair follicle's microbiota). The compositions of the invention comprise the fermented product as described above and other dermatologically acceptable additional components. The definition “dermatologically acceptable” as used herein indicates that the compositions or the described components are suitable for application on human skin or on the scalp without toxicity risk, incompatibility, instability, allergic response, and the like. The fermented product as described above may be used in compositions appropriately formulated in an acceptable dermatological vehicle. The dermatologically acceptable vehicle can be an aqueous or hydroalcoholic solution, an emulsion water-in-oil, an emulsion oil-in-water, a microemulsion, an aqueous gel, an anhydrous gel, a serum or a dispersion of vesicles. The compositions may include, for example, vegetable oils, such as oil sweet almonds, olive, sunflower and/or beeswax. Moreover the compositions may include hyaluronic acid and/or glycolic acid and/or serum and sun protection factor. The compositions may include different additional ingredients, which can be active ingredients, functional agents or agents normally used in cosmetic products, products for personal care or for pharmaceutical use of topical or transdermal type. The decision to include an additional ingredient and its choice depends on the specific application and product formulation. A wide variety of additional ingredients that can be added into the compositions of the invention are mentioned in CTFA Cosmetic Ingredient Handbook, 10th edition, 2004 (published by Cosmetic, Toiletry and Fragrance Association, Inc, Washinghton, D.C.). Additional ingredients include amino sugars, vitamin B3 derivatives, dehydroacetic acid (DHA), phytosterols, salicylic acid, ascorbic acid and derivatives, sunscreens, keratolytic, anti-oxidant and anti-radical agents, antimicrobial agents, antibacterial agents, antifungals, thickening agents, anti-odorants and fatty acids. The compositions of the invention may comprise at least one vehicle acceptable from the dermatological point of view, which is chosen accordingly to the final formulation of the product. Preferably the vehicle is present at a concentration between 50% and 99.99%, more preferably between 60% and 99.9%, even more preferably between 70% and 98% by composition weight. The compositions of the invention may be obtained by any suitable method known to those skilled in the art for the manufacture of a cosmetic and/or dermatological composition. Said compositions may for topical applications. They may be present in different formulations, including creams, lotions, ointments, milks, gels, emulsions, suspensions, anhydrous preparations, lotions for scalp treatment, creams or lotions for skin or hair care, sunscreens, transdermal patches, spray formulations and soaps. Said compositions may be also in the form of lip sticks or, in general, of products suitable for face makeup. The invention will be illustrated by means of non-limiting examples with reference to following figures.

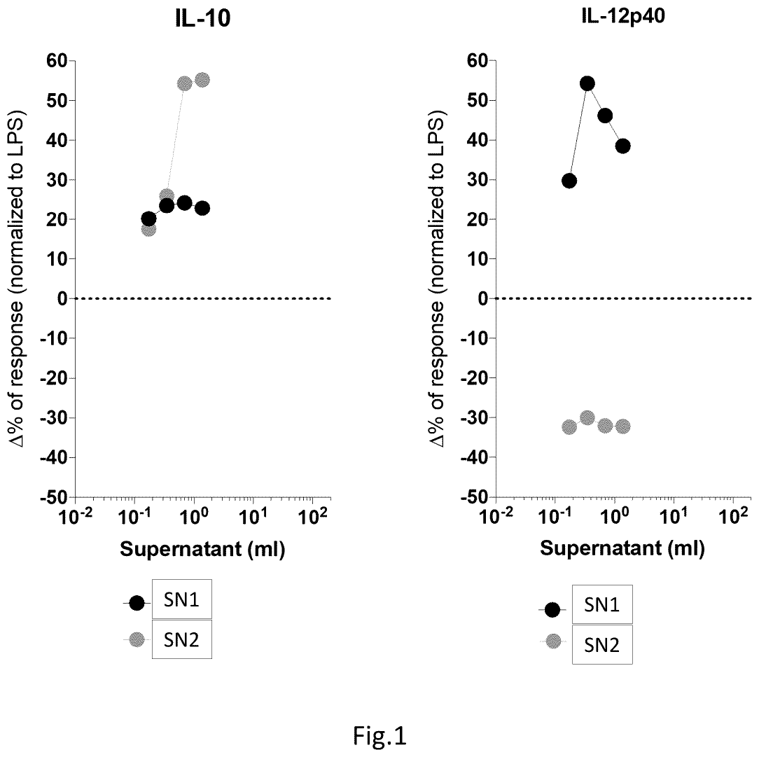

FIG. 1 PBMCs were stimulated with LPS (100 ng/ml) and treated with SN1 (supernatant of first fermentation) or its control (broth medium), or SN2 (supernatant of second fermentation) or its control PBS for 24 h. Concentrations of IL-10 and IL-12p40 were determined by ELISA. The delta % of response (normalized to LPS) was calculated as the net effect of each fermented product versus each relative control medium.

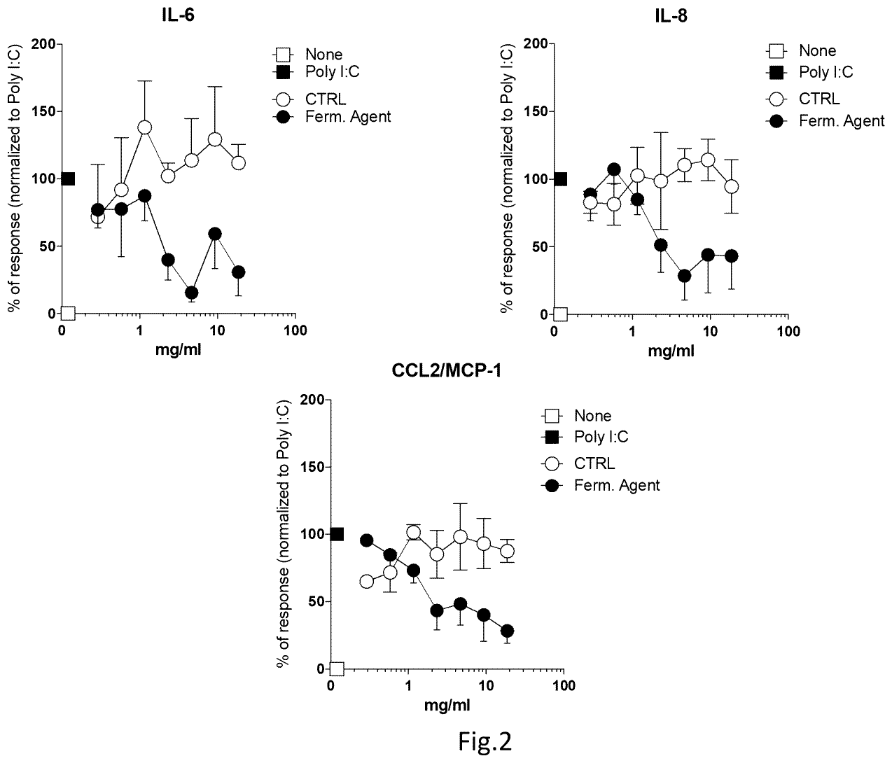

FIG. 2 HaCat cells were stimulated by Poly I:C (1 μg/ml) and treated with the Postbiotic and the relative control containing Mannitol and sodium lactate for 24 h. IL-6, IL-8 and CCL2-MCP-1level were determined by ELISA. Mean of 3 experiments it is shown.

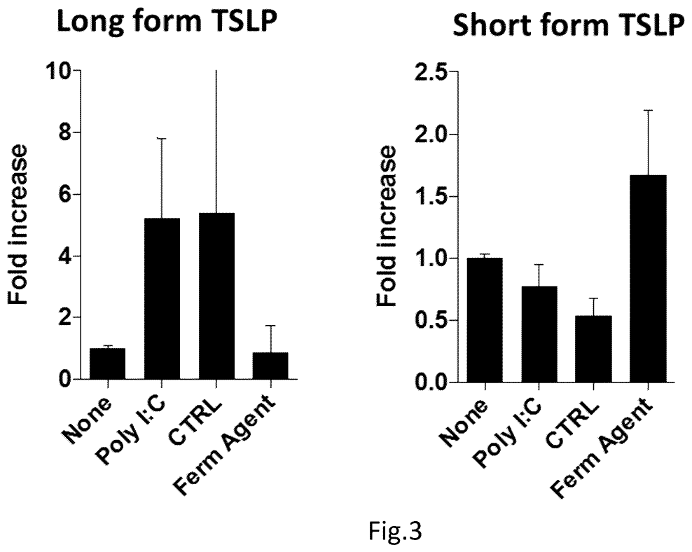

FIG. 3 HaCat cells were stimulated by Poly I:C (1 μg/ml) and treated with the Postbiotic and the relative control containing Mannitol and sodium lactate for 6 h. Gene expression of TSLP forms, ITSLP and sTSLP, were evaluated by qPCR.

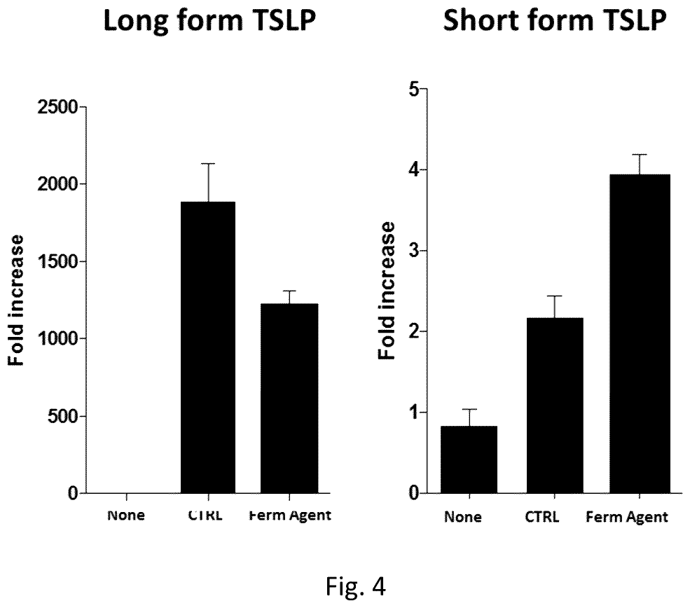

FIG. 4 NHEK cells were stimulated by Poly I:C (1 μg/ml) and treated with the Fermented Agent (Postbiotic) and the relative control containing Mannitol and sodium lactate for 6 h. Gene expression of TSLP forms, ITSLP and sTSLP, and its receptor, were evaluated by qPCR. Statistical comparisons were based used Mann-Whitney U Test. *p<0,01. Untreated and Fermented Agent (Postbiotic)-treated cells were compared with postbiotic Control. CTRL/Fermented Agent=1 mg/ml.

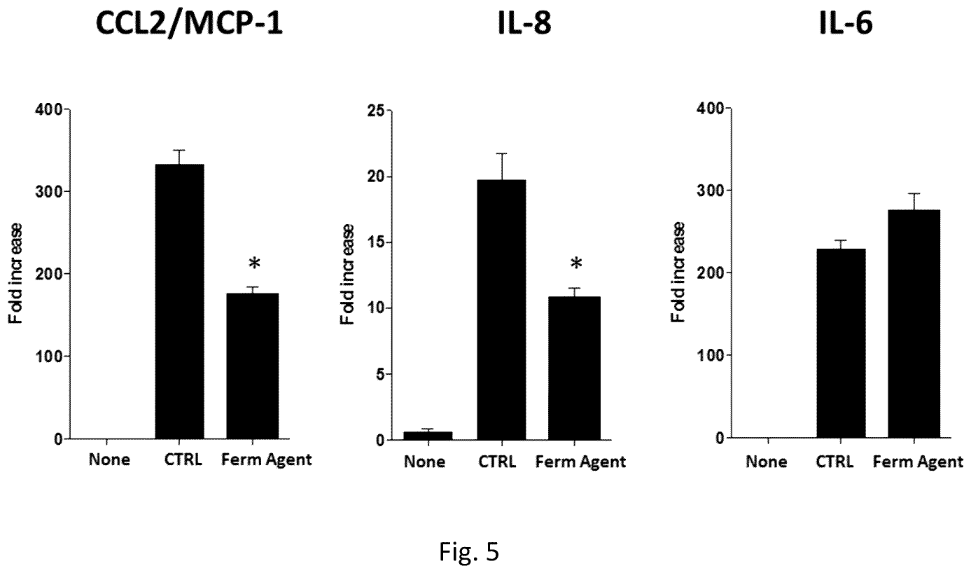

FIG. 5 NHEK cells were stimulated by Poly I:C (1 μg/ml) and treated with the Fermented Agent (Postbiotic) and the relative control containing Mannitol and sodium lactate for 6 h. Gene expression of CCL2/MCP-1, IL-6 and IL-8 were evaluated by qPCR. Statistical comparisons were based used Mann-Whitney U Test. *p<0.01. Untreated and Fermented Agent (Postbiotic)-treated cells were compared with postbiotic Control. CTRL/Fermented Agent=1 mg/ml.

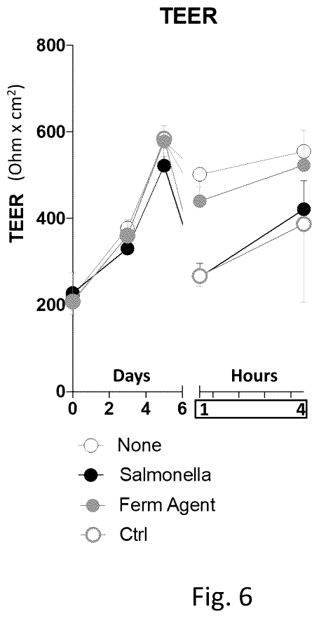

FIG. 6 Caco-2 epithelial cells were stimulated with Salmonella SL1344 (6×108 CFU/well) for 1 h and 30′ and treated with Fermented Agent (Postbiotic) and the relative control containing Mannitol and sodium lactate for the next 4 hours (CTRL/Fermented Agent=10 mg/ml). Trans-epithelial resistance was measured by chopstick electrode.

### EXAMPLES

### Material and Methods

### Fermentation Process

First Fermentation: Lactobacillus paracasei Biomass Preparation

An inoculum of Lactobacillus paracasei strain CNCM I-5220 of Institute Pasteur collection, were grown in MRS medium at a temperature of 37° C. then gently stirred to avoid oxygenation of the culture medium, as MRS medium. The biomass is then allowed to grow for about 12 to 36hours, preferably for about 24 hours, until the desired concentration of lactobacilli is reached comprise between 2×10\hat{ }8 and 5×10\hat{ }10 CFU/ml. The obtained bacterial suspension is centrifuged at 3500 rpm for 10 minutes to separate bacteria (biomass) from the supernatant to obtain SN1.

Second Fermentation: Lactobacillus paracasei Supernatant Preparation (Postbiotic)

The lactobacillus biomass, as defined above, is transferred to a minimum solution (saline, phosphate buffer, H2O, etc.) with or without sodium lactate (appr. 5 5 g/L) and allowed to ferment for 12 to 36 hours, preferably for about 24 hours with or without agitation. The bacteria suspension is then centrifuged to separate them from the, second fermented product (SN2, here also referred to as “fermented product”), and the latter is used for the preparation of the formulation for topical use.

The “postbiotic” used below, is the fermentation product (SN2) mentioned above obtained in the presence of sodium lactate.

### Preparation of the Topic Formulation

The fermentation product obtained from the first and/or the second fermentation (i.e. SN1 or SN2) is used in a topical formulation diluted to 0.01-10%, preferably to 0.5% in cream base made up with e.g. 60 g vegetable oil such as sweet almond oil, olive oil, sunflower oil and 3 gr of beeswax, hyaluronic acid (0.1%), glycolic acid, serum and sun protection factor (Spf20). The % of fermented product also depends on the concentration of bacteria obtained during the second fermentation.

### Fatty Acid Analysis by GC/MS

250 ml of samples (of SN1 or SN2) were lyophilized and suspended in 500 μL. Trans-esterification was carried out by adding 850 μL of chloroform, 150 μL of H2SO4 and 1 mL of methanol. Thus, samples were heated at 100° C. overnight and to stop the reaction and eliminate water 2 mL of a 100 mg/mL sodium bicarbonate solution and 1 g of 99% bicarbonate were added. Subsequently, fatty acids were extracted using chloroform. 1 μl of the extract was analyzed in GC MS using a C18 30 m column. The fragmentation spectrum interpretation was performed by comparison with theoretical spectra in the NIST database. The area of the peaks was interpolated with a calibrator (FAME, Sigma Aldrich) to perform a quantitative analysis.

### Peptide Analysis by MALDI-TOF

200 ml of samples (of SN1 or SN2) were lyophilized and suspended in 4 mL of 1% formic acid. 1 mL of concentrated samples were analyzed by HPLC-UV on RP C18 column using 0.1% formic acid (eluent A) and acetonitrile containing 0.1% formic acid (eluent B) as eluents (flow rate 0.5 mL/min; absorption wavelength 220 nm). Fractions were collected and analyzed by MALDI TOF mass spectrometry in positive ion mode, using α-Cyano-4-hydroxycinnamic acid as matrix. Signals of interest underwent to tandem mass spectrometry analysis using MALDI TOF/TOF. The fragmentation spectra were collected and interpreted. The peptide sequences confirmed by alignment with the BLAST program.

### Peripheral Blood Mononuclear (PBMC) Cells and Stimulation Conditions

Buffy coats were obtained from healthy donors having signed an informed consent for research use. PBMC were separated with Ficoll (GE Healthcare) gradient centrifugation and then resuspended and cultured in RPMI 1640 medium (Lonza) containing 10% fetal bovine serum (Gibco), 1% Glutamine, 1% pyruvate, 1% non essential AA and 1% Penicillin-Streptomycin. PBMCs were incubated with Lipopolysaccharides (LPS) from Escherichia coli O111: B4 (Sigma-Aldrich) in the presence or absence of serial dilution of 1.3% v/v liquid supernatant of first fermentation (SN1) or supernatant of second fermentation (SN2) for 24 h. Supernatants were tested for cytokine abundance by ELISA (R&D systems). The delta of normalized response to LPS of each fermented product was calculated by subtracting the effect of the media of fermentation (broth medium in case of SN1, PBS in case of SN2) from effect obtained by treatment with SN1 or SN2.

### Stimulation of Human Keratinocytes in the Presence or Absence of Postbiotic

Human keratinocytes cell line HaCat (Fornasa et al., 2015) was cultured in DMEM supplemented with 10% FBS, 1% Glutamine, 1% Penicillin-Streptomycin. Cells were stimulated with 1 μg/ml of Polyinosinic:polycytidylic acid (Poly I:C) and treated with serial dilution (factor 2) starting from concentration of 20 mg/ml of Postbiotic (i.e. lyophilized SN2 resuspended in water) and its relative control containing Mannitol and sodium lactate for 24 h. Then, supernatant was recovered and levels of inflammatory mediator IL-6, IL-8 and CCL2/MCP-1 were measured by ELISA technique (R&D systems).

### Gene Expression of the two Form of TSLP, ITSLP and sTSLP, was Evaluated by qPCR, After HaCat Cells Stimulation as Described Above, After 6 h

Total RNA was extracted from the cells treated as described, by RNeasy Mini Kits (Qiagen), adding a DNase digestion step to clean the contamination of genomic DNA. lug of RNA was reverse transcribed with the reverse transcriptase enzyme (Im-prom-II-reverse transcriptase, Promega), primers oligo (dT) (Invitrogen). Real time PCR (qPCR) was performed with Fast SYBR Green PCR kit on Applied Biosystems 7500 Fast Real Time PCR System (Applied Biosystems) with 20 ng of cDNA and specific primers for the two forms of TSLP (Fornasa et al., 2015). Gene expression was normalize to the expression the housekeeping gene, glyceraldehyde-3-phosphate dehydrogenase (GAPDH). The relative mRNA quantification was calculated by ΔΔCt method.

### Stimulation of Primary Normal Human Keratinocytes in the Presence or Absence of Postbiotic

Normal Human Epidermal primary keratinocytes (NHEKs, MatTek Corporation) were cultured in NEHK growth medium supplemented with 1% growth supplement (NHEK-GS, MatTek Corporation) and 50 μg/ml Gentamycin. Cells were stimulated with 1 μg/ml of Polyinosinic:polycytidylic acid (Poly I:C) and treated with 1 mg/ml of Postbiotic (i.e. lyophilized SN2 resuspended in water) and its relative control containing Mannitol and sodium lactate for 6 h. Then, gene expression of the two forms of TSLP, ITSLP and sTSLP, and inflammatory mediator IL-6, IL-8 and CCL2/MCP-1 was evaluated by qPCR. Total RNA was extracted from the cells treated as described, by RNeasy Mini Kits (Qiagen), adding a DNase digestion step to clean the contamination of genomic DNA. lug of RNA was reverse transcribed with the reverse transcriptase enzyme (Im-prom-II-reverse transcriptase, Promega), primers oligo (dT) (Invitrogen). Real time PCR (qPCR) was performed with Fast SYBR Green PCR kit on Applied Biosystems 7Flex Fast Real Time PCR System (Applied Biosystems) with 20 ng of cDNA and specific primers for the two forms of TSLP (Fornasa et al., 2015). Gene expression was normalized to the expression the housekeeping gene, glyceraldehyde-3-phosphate dehydrogenase (GAPDH). The relative mRNA quantification was calculated by ΔΔCt method.

### Stimulation of Human Epithelial Cells in the Presence or Absence of Postbiotic

Caco-2 cells (human epithelial colorectal adenocarcinoma cells) were maintained in DMEM supplemented with 10% FBS, 1% Glutamine, 1% Penicillin-Streptomycin. Experiments were performed seeding 6×10⁴ cells/well on polycarbonate membranes (Transwell 6.5 mm in diameter, 5 μm pore size) (Costar Corp). Caco-2 cells growth were monitored by measuring the trans-epithelial electrical resistance (TEER) until confluence by chopstick electrodes (Millicell-ERS, Millipore). TEER is a widely accepted quantitative technique to measure the integrity of tight junction dynamics in cell culture models of epithelial monolayers. Then, the cells were stimulated with 6×10⁸ colony forming units (CFU)/well Salmonella typhimurium during 1 h 30′ and then the cells were treated with Postbiotic (i.e. lyophilized SN2) and its relative control containing Mannitol and sodium lactate for the next 4 hours and the TEER was measured to evaluate the integrity of the monolayer at endpoint.

### Results

### Chemical and Functional Characterization of Fermented Processes Supernatant

In order chemically characterize the supernatants a quantification of fatty acid was performed by Gas Chromatography with Mass Spectrometric Detection (GC/MS). The inventors found that the fermented product (supernatant/SN) derived from the first fermentation process (SN1) and from the second fermentation process (SN2) had a different fatty acid profile (Table 1). Both supernatants contain oleic acid which is reduced in SN2 compare SN1. These results indicate that the composition of the supernatants is different. Moreover, supernatant was analyzed for bio-peptides content by MALDI-TOF technique. The inventors found that the SN1 and SN2 had also a different bio-peptides profile. In particular, for SN1 was not detected any signals by MALDI/TOF compare to SN2 (Table 2a) and relative fragmentation (Table 2b). These results further confirm that the components present into the two supernatants are different. In addition, the inventors have evaluated the immunomodulatory proprieties of the two supernatants on LPS-stimulated PBMC. Peripheral blood mononuclear cells (PBMC) are a heterogeneous cell population that includes myeloid as well as lymphoid immune cells. Supernatants, SN1 and SN2, were tested for their ability to modulate cytokines release in particular IL-12p40 and IL-10 by LPS-stimulated PBMC, which is a stimulation that mimicking innate immune activation. The inventors found that SN1 (fermented product derived from first fermentation) and SN2 (fermented product derived from second fermentation) have different immunodulatory profile.

The net effect of each fermented product was calculated subtracting the contribution of the relative fermentation media (see material and methods). The inventors found that SN2 led to a reduction of IL12-p40. Conversely, SN1 induces IL12-p40. In addition, SN2 significantly increase secretion of IL-10 (FIG. 1) compare to SN1. Those results indicate that SN2 has immunomodulatory proprieties characterized by the reduction of proinflammatory cytokine IL-12p40 and increase of anti-inflammatory cytokine IL-10 controlling activation of both innate and adaptive immune system compartment. These result, clearly demonstrate that the two supernatants are different both in term of chemical composition that biological/immunomodulatory function.

### The Effect of Postbiotic on Keratinocytes

The epithermal epithelial cells, keratinocytes, play an essential role to the formation of cutaneous barrier and in the orchestration of innate and adaptive immune response to external stimuli. Alterations of these responses are responsible for several epidermal diseases. In particular, increased secretion of IL-6 and IL-8, cytokines by keratinocytes, promotes a reduction of cornified-envelope-associated protein such as filagrin, loricrina and keratin (Noh et al., 2010) (Jin et al., 2014). Moreover, the release of chemotactic factors such as CCL2/MCP-1 will recruit cells of the immune system, amplifying the inflammatory response. Such immune response in combination with the long inflammatory form of TSLP, lfTSLP, and with an altered release of the constitutive form, sfTSLP, will favour the establishment of an allergic response, Th2, of itchy manifestations and a reduced antibacterial activity. The inventors have stimulated a human epidermal keratinocytes cell line, HaCaT, widely used and representative of primary human keratinocytes, with an inflammatory microbial derivative that mimics the double-stranded viral RNA, the Poly I:C, for 24 hours. The aforementioned stimulation induces the release of inflammatory cytokines as IL-6 and IL-8 and of chemotactic factor CCL2/MCP-1 (FIG. 2). The inventors have found that the presence of the Postbiotic during the stimulation significantly reduces, in dose-dependent manner, IL-6 and IL-8 cytokines and chemotactic factor

CCL2/MCP-1 levels (FIG. 2). The inventors have also stimulated HaCaT cells, as described above, for 6 hours, and observed an increase of RNA level of the lfTSLP (inflammatory) and not of the sfTSLP (homeostatic) (FIG. 3). The inventors have discovered that the presence of the Postbiotic during the stimulation reduces the levels of the cytokine lfTSLP (inflammatory) and increases the levels of the cytokine sfTSLP (homeostatic) (FIG. 3). These results indicate that the postbiotic (fermentation product of L. paracasei), acts on keratinocytes by modulating the release of immunological mediators, favouring the re-establishment of skin homeostasis status. The inventors have also stimulated primary human epidermal keratinocytes, NHEK, with an inflammatory microbial derivative that mimics the double-stranded viral RNA, the Poly I:C, for 6 hours. The inventors have observed an increase of RNA level of the lfTSLP (inflammatory) and the sfTSLP (homeostatic) (FIG. 4). The inventors have discovered that the presence of the Postbiotic during the stimulation reduces the levels of the cytokine lfTSLP (inflammatory) and increases the levels of the cytokine sfTSLP (homeostatic) (FIG. 4). These results indicate that the postbiotic (fermentation product of L. paracasei according to the invention), acts also on primary human epidermal keratinocytes by modulating the release of immunological mediators, favouring the re-establishment of skin homeostasis status. The aforementioned stimulation induces the up-regulation of inflammatory cytokines as IL-6 and IL-8 and of chemotactic factor CCL2/MCP-1 (FIG. 5). The inventors have found that the presence of the Postbiotic during the stimulation significantly reduces chemotactic factor CCL2/MCP-1 and IL-8 cytokines expressions without affecting IL-6 level (FIG. 4). These results indicate that the postbiotic according to the invention, acts on keratinocytes by modulating the release of immunological mediators, favoring the re-establishment of skin homeostasis status.

### The Effect of Postbiotic on Epithelial Barrier Integrity

Epithelial cells create a protective layer for both the outside and the inside cavities and lumen of the body. Epithelial cells are connected to each other via intercellular junctions and barrier integrity is essential for the physiological activities of the tissue. The tight junctions create fusion points between epithelial cells, that regulates diffusion and allows both of these cell layers to form semipermeable cellular barriers that separate apical (luminal) and basolateral (abluminal) sides in the body, controlling the transport processes to maintain homeostasis. The inventors have seeded Caco-2 (human colon adenocarcinoma cell line) in transwells and their growths were monitored by measuring the transepithelial electrical resistance (TEER). When the junctions are strong, it is more difficult for the current to pass between the cells, thus TEER is high; otherwise when juctions are compromised the resistence is lower. The inventors tested the protective properties against enteric pathogen Salmonella on monolayer of Caco-2 cells on transwells. The cells were infected with 6×10⁸ colony forming units (CFU)/well Salmonella typhimurium for 1 h 30′ and then the cells were treated with Postbiotic (i.e. lyophilized SN2) and its relative control containing Mannitol and sodium lactate for the next 4 hours. During all the experiment TEER was measured to evaluate the integrity of the monolayer. The stimulation of Caco-2 cells with Salmonella typhimurium causes a monolayer breakage thus registering low values of TEER. The inventors found that Postbiotic was able to maintain the monolayer integrity after the damage caused by Salmonella typhimurium insult (FIG. 3) shown by high values of TEER that were similar to the unstimulated cells.

### Tables

TABLE 1

| | | mg/L | | | mg/L |
|---|---|---|---|---|---|
| SN1 | oleic acid | 7.98 | SN2 | 7 hexadecenoic acid | 5.12 |
| | decanoic acid | 1.30 | | tridecanoic acid | 5.42 |
| | benzopropanoic acid | 2.69 | | vaccenic acid | 12.23 |
| | citric acid | 13.05 | | oleic acid | 3.21 |

Fatty acid profile quantification of the fermented product supernatant of Lactobacillus paracasei strain CNCM I-5220 obtained from (A) first fermentation (SN1) and (B) second fermentation (SN2) process.

TABLE 2
A

| SN1 | SN2 |
|---|---|
| m/z | m/z |
| NO | 357.07 |
| signal | 423.06 |
| | 373.05 |
| | 407.07 |
| | 439.04 |
| | 489.05 |
| | 505.03 |
| | 538.05 |
| | 557.1 |

B
SN2

| Fractions | m/z |
|---|---|
| 3 | 1193.2 |
| | 855.2 |
| | 756.38 |
| 4 | 524.06 |
| | 542.08 |
| | 664.12 |
| | 756.38 |
| | 644.37 |
| 5 | 756.43 |
| 6 | 610.34 |
| 12 | 1006.8 |
| 13 | 1393.2 |
| | 1153.77 |
| | 1033.68 |
| | 707.28 |
| | 855.54 |
| 15 | 1153.76 |
| | 1393.8 |
| | 1033.65 |
| | 855.34 |
| 16 | 1153.76 |
| | 1393.8 |
| | 1033.65 |
| | 855.34 |

C
SN2

| Fractions | peptides | sequences | Protein |
|---|---|---|---|
| 3 | 855.2 | PDLKDVG (SEQ ID NO: 19) | riboflavin byosintesis protein |
| | 1193.2 | LEIHLNAAQ (SEQ ID NO: 20) | type 2 isopentenyl-diphosphate |
| | 1193.2 | GTNFATIENA (SEQ ID NO: 21) | Delta-isomerase |
| 4 | 756.43 | GTDTRAE (SEQ ID NO: 22) | riboflavin byosintesis protein |
| 5 | 756.43 | GTDTRAE (SEQ ID NO: 23) | riboflavin byosintesis protein |
| 6 | 610.34 | YINAM (SEQ ID NO: 24) | type 2 isopentenyl-diphosphate |
| | | FMWQ (SEQ ID NO: 25) | Delta-isomerase |
| | | HLIQT (SEQ ID NO: 26) | |
| 12 | 1006.8 | TAFAALTEL (SEQ ID NO: 27) | riboflavin byosintesis protein |
| 13 | 707.28 | MEANML (SEQ ID NO: 28) | riboflavin byosintesis protein |
| | 1393.8 | NDYEKIAMGLLS (SEQ ID NO: 29) | |
| | 1033.6 | FIPDLKDV (SEQ ID NO: 30) | |
| | 1153.7 | MPTEQ----- (SEQ ID NO: 31) | tetratricopeptide repeat protein |
| | 855.2 | PDLKDVG (SEQ ID NO: 32) | riboflavin byosintesis protein |
| 15 | 855.34 | PDLKDVG (SEQ ID NO: 33) | riboflavin byosintesis protein |
| | 1393.8 | NDYEKIAMGLLS (SEQ ID NO: 34) | |
| | 1033.6 | FIPDLKDV (SEQ ID NO: 35) | |
| | 1153.7 | MPTEQ----- (SEQ ID NO: 36) | tetratricopeptide repeat protein |
| 16 | 855.34 | PDLKDVG (SEQ ID NO: 37) | riboflavin byosintesis protein |
| | 1393.8 | NDYEKIAMGLLS (SEQ ID NO: 38) | |
| | 1033.6 | FIPDLKDV (SEQ ID NO: 39) | |
| | 1153.7 | MPTEQ---- -(SEQ ID NO: 40) | tetratricopeptide repeat protein |
| | 1006.8 | KLTEATVM (SEQ ID NO: 41) | tetratricopeptide repeat protein |

Peptide profile of the fermented product supernatant of Lactobacillus paracasei strain CNCM I-5220 (first fermentation (SN1) and second fermentation (SN2) process) obtained from MALDI TOF mass spectrometry (A). Signals of interest from SN2 were profiled by MALDI TOF/TOF (B) mass spectrometry. (C) Peptides sequence profile of the fermented product supernatant of Lactobacillus paracasei strain CNCM I-5220 (second fermentation (SN2) process) obtained from MALDI TOF mass spectrometry. Fragmentation spectra of peptidic origin were interpreted and sequences confirmed by alignment with the BLAST program.

### Sequences

Included in the present invention are also nucleic acid sequences derived from the nucleotide sequences shown belove, e.g. functional fragments, mutants, derivatives, analogues, and sequences having a % of identity of at least 70% with the below sequences.

In the following are disclosed five gene sequences, representing specific core genes of L. paracasei and L. casei species.

SEQ ID 1
ATGAATCAAAAAGCTTTGAATCAATTTCCTGAACTTACCTACACAGAACA
AGTGTCGGTTGTTGGCGGCGATTTGTCAGTCGAAGTCATCATGAAAGGTA
TCTTCACCGGTATCTTTGATGCTGGGTACCAAGTGGGTCAGTCAATCGCA
AAATGGGTTAAGTAA
SEQ ID 2
ATGAAAGGTAAGCGGCAGCACTTACTTTTATATTTTGTTCTGGGTATGAT
GACAGGACTGGTGACGGCAGCGATTTTTCATATCATTTATGCCTGGCTTT
TTCATTGA
SEQ ID 3
ATGCCCAAAAGGGTCGATCAACATATACGTTCACGCCTTAAAGGCTTTAC
TTTAATTGAAGTGGTGGTCAGCCTGATTTTACTTGCGGCGGTCATGCTGT
TATGGCGACCGGTTTTATTGCATGTCACGCGGTTCACGCTTCAAGACCAT
GTGCTAATCACGTCATTGCAAGCAGAGCATGACTTGCAAATGTTTGTACG
AGATAAAAAGTTGCGGTCTGTGGCCTTAATGTCGGTAAGGGTGAGAAGTC
CCGAGAAAGCTTACACGATCAATTTTTATCAGACCAAACATTTTCGCGGT
ATGGTTCGTGTGATGGGATCTGAAAATGGGCATATGCCATTATTTACGCA
TCTAACCGGTGTCAATTTTAGCAAGGTAGCTCAAGGCTTTCGCTATCGTC
TGTATTTGACGACTTCGCAGAAGATTGACGGTGGGGTGCAAATCGATGAA
GATACGCGGTAG
SEQ ID 4
ATGGCCGCTGATTTCACCCAATTACAACAAGCCATTCGCTTGCTCAATGC
CCATACTCGAGCTGCTGATGAGCAAGCGTGGCAAGTGCTTTTTGATCGTT
GGCTGGCAACTTTATCCTCTGAAACTCGCCGGCAAATGCAAACAGTTCGG
TTTAATCATGCCCAATTGACGTTACTCACAACGCTGGATCAAAGCAGTCG
CAAACAACTGCGCAATCAGGATTTAACCGCTGCTGTTCCGTTCTCACAAG
GCCTAGTCTCACGCTATGTTGCTCGCCTTGTTCAATTAAACTTGCTGACA
AAATTATCCTTGCCCGACAATCGCAAGGCCTACATTGTTGCACTAACTCC
GCTTGGTCAACAAGTCGCTGCCTTACATCAGCAAATGCATCATCACACAA
ATGCTCAACTCGCCTCTGTACTTCATACCCTTGATCCACAAGATGTTCAA
ACTACCATTCAGGTACTCACAAAACTGACGGCTCAGCCTTTACATCCCAA
GTCTTAG
SEQ ID 5
ATGGGCGGTGTCATTTGTTACGCGGTGCCGGTCTTTTGGAAAAGAATACT
TCGCAGACACCTGATTCACGAGATTAAGACCCTGAATCAAGGATTGCAGT
TATCAAGCAAAGCCATGAGCCAATTAATTGATCCGGAAAATCCTTATATG
GTATTTGCTGATGAAAATGGTGAACTGGATTTTTCATTTTTGTGGCTAGG
CAACTTGCGTCAATTGAGGCGTGAACTGCGTCTAATTAAAGAACAGAAAG
CTAGGGTTTGA

In the following are disclosed unique sequences of L. paracasei B21060 with respect to the L. paracasei species publicly available in NCBI databases. SEQ ID 6 to 8 refers to gene sequences, whereas SEQ ID 9 to 18 refers to genome DNA sequences.

SEQ ID 6:
ATGAGTAAATATAAAGTTATTATTTGGGGATTAGGAAACGTTGGTCGTTCCGCAGTGAGAATGATCGC
GGAAAGACAAAATATTTTTGAATTGGTTGCAGCCGTTGACGTTGATCCAAAGAAGTTAGGTAAGGATG
CCGGAGAAGTCTTTGATTTTGACAAAGTCGGCGTCAAAGTTTCAGATGATATTGATGCAGCCTTGAAA
CTTCCAGCTGACATTGTGCTCGACTTTTGCCCAACGGAAATGGACAAACAAGGAACATTCATGCCTT
CTGCTATTCGACTCGCCAAATCGCTCGATGCCGGTAAAAACGTTATTACCACGATTCCGGTATATCAT
GTTCAAGACAGTCAGCCAGAAGTATATGAATATCTAAATGAACATGCTAAAGCACATAATGTTGCTTT
TGTACCATTTGGACTTTTGCCAGGCGATTATGCCTCATATATCCCACTAGTTTTGGCCGGGGCCATG
GGCCACGTGGATAAAATTGTTGTTCAATCCGGTGAAGATGACTGGCACAACACATCAGGCTGGGTC
GATGTCTTCTCATATGGCGGCGATATCAATAAATATCCAAAACCAGACTCAGACGAAGATCTCTTGGC
TAAGTTCATTTATGCTTATTATTCATCCGGCGTATACGAGATGGCCGATAGGATCGGTCTGAAATATG
ATACCTTCAAACCAGAGCATGAAGTCTTCACTGCACCCAAAGATTTGGAAACGATCAAGGGTACAGT
CAAAAAGGGCAGCATTTATGCCCACAGATTTACCATGGCACTTTACAACGGCAACGAACAAGTAGCC
GCCTTAAGATATGTTCATAAAGTTGATAATAAAGAGACACCAGAATTACCGATCAATAATACGATTCAT
ATTGAAGGCTTGCCGTCAGTCGATGCGCAGATCGATGGATTGATCCCAGAAAGAGAAGGCTACGTTT
CATCAGCCGCTCCAGCAGTCAACTTGATCCCTAGCATTCTCGAGACCGACAAGACAGGTTATGTTGA
AGTCTGCGACCTTCCAGTAGTGATTGCCAGGCCATTGGATATTGGCGCAAAAAAATTAGTCTAG
SEQ ID 7:
ATGGCAACCTATTCGCAGATAGAACTAGACATAATCAAATCATTTAAAGGGCTGATGAAAGACCATGA
ATTCACTGAGATCTCAATTAAAATGATCGCTGAAAAAGCCGATATCACTCGACGCGGCTTTTACAATC
ACTTCTTAGATAAATATGATCTTGTCAGTACCATCTTTGAGCATGATCTTTTTCCAACAGTCATCAGTT
TGACGAATATCAATGACTGGGATCAAGGGTCGCTGTTTATCGTGAATTATCTCCAAGACAATCGCGA
CTACTATAAAAAATTGTTGTCGCTTGAAGGACAAAACTGTTTACAGACAGACTTTTATAAATTGACTGA
GATGCAGATTGGGATCTTGATCCCAGAAATATTGGTCGGTAGGAAAATTTCTGACGAAGATCAGGCA
TTTTTAAGCGATTATTATTTTCACGCTTATATGGGACTGACTACCGAATGGGTCAAAGGTAAATATGGT
TTTTCAACTCAGGAGTTCGTTAAACGGTGGAAAGCCTTACTCAATAATTCAATGCATAATTATCTGGA
CAACTACGCTCGATGA
SEQ ID 8:
ATGAGTAAATATAAAGTTATTATTTGGGGATTAGGAAACGTTGGTCGTTCCGCAGTGAGAATGATCGC
GGAAAGACAAAATATTTTTGAATTGGTTGCAGCCGTTGACGTTGATCCAAAGAAGTTAGGTAAGGATG
CCGGAGAAGTCTTTGATTTTGACAAAGTCGGCGTCAAAGTTTCAGATGATATTGATGCAGCCTTGAAA
CTTCCAGCTGACATTGTGCTCGACTTTTGCCCAACGGAAATGGACAAACAAGGAACATTCATGCCTT
CTGCTATTCGACTCGCCAAATCGCTCGATGCCGGTAAAAACGTTATTACCACGATTCCGGTATATCAT
GTTCAAGACAGTCAGCCAGAAGTATATGAATATCTAAATGAACATGCTAAAGCACATAATGTTGCTTT
TGTACCATTTGGACTTTTGCCAGGCGATTATGCCTCATATATCCCACTAGTTTTGGCCGGGGCCATG
GGCCACGTGGATAAAATTGTTGTTCAATCCGGTGAAGATGACTGGCACAACACATCAGGCTGGGTC
GATGTCTTCTCATATGGCGGCGATATCAATAAATATCCAAAACCAGACTCAGACGAAGATCTCTTGGC
TAAGTTCATTTATGCTTATTATTCATCCGGCGTATACGAGATGGCCGATAGGATCGGTCTGAAATATG
ATACCTTCAAACCAGAGCATGAAGTCTTCACTGCACCCAAAGATTTGGAAACGATCAAGGGTACAGT
CAAAAAGGGCAGCATTTATGCCCACAGATTTACCATGGCACTTTACAACGGCAACGAACAAGTAGCC
GCCTTAAGATATGTTCATAAAGTTGATAATAAAGAGACACCAGAATTACCGATCAATAATACGATTCAT
ATTGAAGGCTTGCCGTCAGTCGATGCGCAGATCGATGGATTGATCCCAGAAAGAGAAGGCTACGTTT
CATCAGCCGCTCCAGCAGTCAACTTGATCCCTAGCATTCTCGAGACCGACAAGACAGGTTATGTTGA
AGTCTGCGACCTTCCAGTAGTGATTGCCAGGCCATTGGATATTGGCGCAAAAAAATTAGTCTAG
SEQ ID 9 Position 102558 . . . 102986
AAAAACGGCTTAGAACGCTCATATTTGCGTTCTAAGCCGTTTTTATCAGCATAGGTTCTTGACACCAA
TAAACATCTTTAGTAATTGATCAAATTTAGGCAATGTGCTTTTGTCGGTGAATGGCGATAGCCCTACC
GAAGCTTCAGCTGAGGTTCTTCTGAGCCACGCAAGCGAAGCGCGCTAGGGCAAGCCAACGGCGCG
CAGGCGAAGCCGGAGTTAAATGTGGCGCAGCCACACCTTTTTAGGGAGCAACGCGACCAGAATTTT
GTATGGGGTTTGGGAAGAGGTTCTCCCCAAGGTCTTTTGTGGTTATTAACAAGCAAAACACAAACAC
AAGCCTCGCGCGCGTTATATATACTTCTAAATACTTTTAAATACTTTAAGTACTTAGGGAGACGAGAAT
GGCTCAACCACGCGTTTAAATCGACT
SEQ ID 10 Position 103624 . . . 103864
Position
103624 .. 103864
ACGACCTCTCGACCACCCACTGCCTCACCAATCCCCAGGTGAACCGGGCCAAGGGCACTACCGAGC
AACCCGACCCCTATATCCCGGTGGGCGTGGTGAAGCAGACCGATGGGGGCATCGTGGTGCGGGGC
GCGCGGATGCTCTCCACGCTGCCCACGGOGGATGAGCTTTTAGTCTTCCCCAGCACTTTGCTCAAA
GAAGGGCCGGGAGCCGACAAGTACGCCGTGGCCTTCGCCATCCC
SEQ ID 11 Position 254291 . . . 261674
ATTGATCGCCTCCGGGTCACTTATATGTAACTAATAATACTCCCTTCTCTCTTTAGTTACAATAGGGTA
CAGCCTATCGAATCACTTACGCTTCCACTTTGAGATAACTTTTCGTTATTATTTATCAACGGCATTAAC
GATATCATTAACTGTTTGCAATGCATCGCTCAGTACACTAATTGGTGCTTGTTCAATATACTGCATGTG
TCGTTGTACAAAATCAAAGGTGTGAAATTGTAACGGATTCACGTACCCTTCTATTTTTTCAGTCTGAAT
CGGTACCATCAAGCCTGTTTCAGCTAAGCGATTATGTTTGGCATGCGTGATCGGACAAACCAACGCT
AATCCAGTCATTTTGGCATATTGTTGATTGCTAATAACGAGCGCTGGTCGTCTTTTTTGAATTTCATGA
CCCCGACTTGGCATGAAATCAATACTCACAACATCACCTTTGCGTGGTTGATAATGCCTAGTCCCACT
CACTTGGTAATACCTCGTTTTCTAACGTAATATCTTGTTGGTGTACTTGTTGCTTGTACCAATCACCTT
CAAATGGATTGCGGTGCTTCGGCAGATAAAGAATGCCACCATCATCACGTTGCTCAACTGTAAATTC
AGTTCCATCGGCGATTTTAATACTCTTTGGAATGGTTAATGTAATGGAATTGCCAACCCTTCTTGCTTT
AACTGTCATTGAAATCATCCTTTCGTATACACCGAGTATACACCAGCGCGAATGAATCTGCAACTCTT
GTGCCCTCTTGTGTACAAACACCACTGTCAATTTACTTTTGCCTATTGTGCTTTATCTCTTCTCGTTCT
GTCATTAGTATGCCACCAACACGGCCGACTTCATCCGGCTCACCTTTGATGACGCCTTGAATACCAT
CGATTTGCTTCCAAGTCTTTACTTCCAAGTTCTTCAAAGACTTTGATATCGTTTTTCGATGCCGGCAAA
CACTTCTTCATTCTGATGCCTTTCAAAGAATTCCTCTAGATCATTCATCAGAGATTCTCCTCACTTAAA
CCTAGTTGGCGTCAAATTCCATCTCAGCAATCGAATCTTCTAGGCTGTCCATTACTTCGTATGTTTCA
ATGAAGTTAACCCAGCTGTCATATGGATGTAGTTTGCTTTCTTTAATGCTAGTTCTAAGCTTTTCGTAC
ACGTTGTCAATAAACTGCTCAACCATCCTATTAACCGCACTATTAATAACCGCTTCGTTCCACTGCTTA
GATGCTGCACGCTTTCGATTTTGACTATGCTGATGGGACAGCGCCCGGCTTCTGAACAACGCTACTG
CCCTGCTGTTAGCATTCAGATAGGTTTCAAATTCTTCACGATCCATTACGTTTCCTCCTCAAAATAAGC
CTCATTTCATAGCACAGCTTCAGCAAAAGGCATGTCATCCTACATGCCTTTTTTCTGTTGCTCTTCAAT
ATCAGTATAAAACGTCCTGCCGCTTTAGGCAAACGTATGTTCGCTATTAAGAACATACGTTTGTATAA
TAACTATAAAAGATTTAAAGGAGGTCAATCGTATGGAAAACAATGTCCCGCGTGAAAAATGGCTTTAC
CCTGACCGCTGCATGAAGAAATGGCTGGGCTGGATTCTAAGCGACCATTCCGCCTATATGGAAGAA
GCGGCTATCTCAGAACAACCGGTGCTCCCAAAGCCTGAACAGACACAAGAAACCATTAATGGCGTAC
TCGAAGATGCTTGGCAAAACTCAAAAATTGTCGCAGTTCAAATCGGTACGCCATACGATGATCTTCTG
TTACCGGATATTGAAGGCGCCGTGATTGGTCATTGGGACGCTCAGGTTTATCTACAGCTTAAAACTG
GTGAGATGCAATCCATTAATGCAGCGGACATTCGCAATGTGCAACTGCTCAATCCAGATCGGTGGTG
GGCGTTAGTATGACGACACCATTAGATGATCCAACAAGGTTACCGGTACACGACATTATGTGCATTG
ACTGTAAGTCCTTTTACGCCTCAGTTGAAGCTATCAGACGCGGGATTCATCCGTTAGCCGCCGACAT
TGCTGTTCTCAGCAAAGGTAATTCTCCTGGCGGTTTGGTGCTGGCTGCTAGTCCCAACTGCAAAAAG
CGTTACCACGTAGGACTGAGTACACGCCGTTTTCAGCTAAGGGACGATATGTAGGTAGAACTTGCTG
AACCGCGGATGGCTAATTACATTCGCAAGAATTACGGTATCAATCGTATTTACCGTCAGTTTACTGAC
GATGCTCACTGGTCTCCCTATTCCGTTGACGAGTCCTTTATTGACGTTACCCACCCCCACAATCTCTT
CGGTTCTAATGAAGAAATTGCTACCCAAATACAGAAGAAGGTGTTTGATCAGTTTGGCATTGTCACAA
CAGTTGGCATTGGGCAAAATCCCCTATTGGCAAAATTAGCCCTAGATAACGAGGCTAAGAAGTCAAC
GCCTTGGCAAGCCACTTGGACTTACGATCGTGTGCCAGAAACAATATGGAAACTTGATGACTTGGTT
GATTTTTGGTCGATTGGTAATCGAACTGCCAAGAAGCTTAACGCGATTGGCCTTCATAATCTTTACGA
CTTGGCTCATGCAGACCGCGCCATTCTGCACCAAAGATTCGGTGTTCTCGGTGATGCCATGTACTTT
CACGCATGGGGTATTGATTACTCAGACTTAACCCGCCGCTACTTACCACGGGCCGAAAATAAAGGCT
ACGGCAATAGTCAGGTACTCATGCGTGATTACACTCAGGCAAGGGAGATTGAAGTCATGCTTAGCGA
GATTGCTGATCAAGTGGCTGGCCGAATTCGCCATCACCAAGTCCAAGGTGAGGTCATTAGCGTTGG
CATTGGTTATGCTGATGCAGAAGAAGCTGGCACCTCCGGTTTCGGTGCGCAAATGAAGATTGATCCC
ACAAATCGCACAGACGATTTAATTCGCGCTACTCGATTTCTCTTCCATAGTAAGTGGAACGGACACG
CTGTTAGAAATGTCTCAGTTCGCGTTAATCGCATCAGCCAAGCAAGTACAATGCAACTTAGTCTATTT
GAATCAGCAGAGAAGGAGGAAGCAAACGCGGCTCCTATGCTGTAATTACGGATAAAAGAATCACCAT
CATTAGGTTTTTCGTCTAACAATTTTAGGAAACTTCACTTTCTAGGTCGTAACTTTATTTTTGCAATCTA
GGGTTTTTTAAATATATACATTTTAGCTCGTTTGTGTTTAATATTATAATCACAACTATACCAATGATAA
ATGTCTAACATAAATATACAAACATGTTGACAGAAGCTCTTGAATACGTTTACAATTATTTCGTTCAGG
CGAGCTTTGTTTTTGAAAAAGTATTAATACAAGATAACTAGGTTAGTGGCTGTTGAATTAGGCCCCCG
ATTTCGGGACCACGACAGTCACTTGATACTCGATTTTTATCGTTTGCTGGCTTGATCGTACATTGAAC
GAAATTGGTACAGAAAAAAGAGCTAAGAGCCGCTCCAAATTAGCCAAAACGATTGCGGCGTCAATGC
TTACGGCGATCGTTTCTGCAGTTTTAGCTGTTACCAGATCAAGTCCTAGTTTCCCTTTGATGAAGGCA
AACTCACGCTCGATCTCACCTCGTCGATTTTCGGCTTGTCGGTCTGCCTTACGTTTGGCCGGATCGA
CCTGCTTCGGCCGACGGCCCAATCTAGGACCGCTAAGTTTGATCCCAAGATCTGCGCACAGCCCGA
TATTCGCCCGAGTCCGATAAAGCGTATCAGCCAAGATCTCATCCGGGTATGTACCATACGTGTCAAA
ATAATGGTCGATCGTTGCTGGTAAGTCAGCACTTTCGTTAAACGCATTGAACGCAAACCGTTCAACG
GCCACGACGCCATGACTGATCGATACGTCGATCTTGGGCCCGAATTCGACCGGATCCTTTGCTTTGC
CGCGAATGATCGGTCGGATCGCTGGTTGATCAAGGCTTACGATCCGATCCGCGACTCGGTGAGTGT
GCTGTCGATACATTTCAGTTTGTTGCTCATACAATTTTCGAATGATCGTTAATCGTTGTGTCTGCCGTT
GATTCAATTGCCCGCCTTGTGCTTGCAGTTCTTTGACGTAACGCAAGTCACGTCGGATGTACTGTAAT
TGAGCCTTGATCTGCTTATGGGTCGTTTTCACCCAACGGGGGGGTTTACGTGAAAAGGCGGTCCAC
GTTTGGTGGGCCTTGCGCTTATAGGTACGCGGCGGTTTGACCGCTAATTGCTTGGCCATGGCTGCG
ATGAATCGCTCTAAATTGAGCCGCGCCTGATTGAGTAGCTGCGTATCCTGCGGATACTTGATCTTTA
CTGGGACCGCAGTCGCATCAGTGATCAAGATCTTCTGATGGCCAAGTTTAGCTTGGAGGCGATCGC
GGACAAAATCGCTAATGATGTTCGTGATCAACTCGGAAAGCGGCGCGATCCGGCGCCTGAAATAGG
ACAGCACCGAAAATGAAAACGGTGCTTGCGGCTGATACTCTGGCAGGCCAATAAAATACTGATAAGC
CGGTGTATCGCGGATCGCTGCGACTAACTCACGGTCCGATAGCTGAGTGCGCTGCTTGATCAGTTG
GGCGCCATAAAGCAGCCGAAAGGGTTTACCTGCCCATCCTAAGTTAGACGGGAAAGCCAATTGGTA
CGCCTCTTCTAGTTGCGGCCACGGAACTTGGTCGGCCAGTTGGACCCACTCGTTATCTGGACTTAAT
GGGGTGCTTAAGCCGCTACCAAACGATTTGATCGATAATTGAACGGCTTTTCGACGATAAACCATGA
TCCATGCCTCCGATAGGGTCGTGTCAAATGCAAACGAAATGAGCACGATCCGTAAATTCATATGCAT
TCATTATACGACGATAACGGGTTCAACTCGCATCAAATGTGGTTATATCAAATTATTCAACAGCCACT
AGGTTAAGATCTTCATTTAAGTGATATTCATTTGCAAGCAATTGAAAATTACTCATCACGAAGAGGATT
TCATTGGCCATATTGGATAGCACGCAAATCACTTGCTTTAAGAAAATCAGTTCCTTTAATGAGTCTCTT
AAAGGACGGGGCTCTCACTTGTACTCACAATCAATGTTAACTGGAGATCAACAATATGGTCATAGATT
CCCATAATAACATTGACTTGACTATCTAAAAGAGGCTTCTAACTTTGATATTGGTGGGGTTATTGGTT
GCTTGGCTGTAAGCAGATAATCTTAACTTGGGTTATTTTCATTGTGTTGTAAAGACATTTGTTATAAAG
GCCGAAGTTATCGCTTTGACTTGTAATAAATTATTTTTGATTGAGATATCAGAAAATAAACGGGGGATA
ATAATGAAAAAGATTATTAGGATTGTTCTTTGTGTTGTTAGTTGCGTTAGTATCATGGTCGGATCGCTT
GGGTTCTATTCAACTCCAAAGATCGTTAAAGCCGACAGTACATCTGTTACGGATGTCGACATTAATAC
CTATATTTCTAGCATGACACTTGATCAAAAAATTGGACAAATGTTTGTAGCACGAACCTCACAAGATA
CTGATAAAGCTCGTGCTGATATAGCAAAATATAATCTTGGCGGGCTGATTGTTTATGGTGTTGATTTC
ACTAGTGTTAAAGGGACAACAGCTACAGAAGCTCAGAATAACTTCAAGATGAAGATGCAAGGCTTTC
AAAACTCGGCAAGTCTGCCACTATTGATTGGTGTTGATCAAGAAGGAGGGGCAGTCTCACGCTTATC
ACAAAATCCTCTAATTGCCAACGGCAGAAGTTTTCCTTCACCACAAATGGCTTATGCTAATGGTGGAA
TGACCAATGTAACAAAAGAAGCTAGTGAAGTCGGAACTATTCTAAAAAATCTGGGCATTAACTGGAAC
TATGCACCAGTTGCCGACAGTACGCCTGACACCTCTAGTTTTATTTATGGTAGAACCTTTGGTCAAGA
TTACTTGGCTACTGCAAACTATATTACGAATGTGATCCCTGCGTGGCAAAATGCTGGCATTGCCGCA
ACTCTCAAGCATTTCCCTGGTTATGGATCCGCGATTGATACGCATACGGATTTTGCAGTCGTTACAAA
GTCTAAGGAGGATTTTGAAAAAGAGGACTTGCTTCCCTTTAAGTCCGGTATTACAGCAGGGGCAGAT
TCTGTAATGATTGCACATATAGTAATGCAAGCTGTTGACCCAGTGTATCCAGCATCATTATCACGGAA
GGTCGTTACCGATTTGTTGCGTAATGAACTTGGGTATAATGGCTTAATAATTACCGATGCATTGGAAA
TGGGGGCCATCAAGCAATTTGCTCAAGAACATGATCAAGTTCCTGTTGATGTTCTTGCTGTTGAAGCA
GGGAATGATTGCATCATGAATAACGATTATGAAACCGCTATTCCACAGATTCATGCAGCAGTAACTAA
TGGAACTATTAAGGAATCAGAAATCAATGAACACGTTTTCCGTATTCTTGATCTCAAACGCAAATTAG
GGTTGTTAACTAAAGGACAACTTCAGCAAAAAAAAGTTCAGGTTGACAATGTTTCCTACAGCAGTGAC
AACAAAAAGGCAACTGTGAGTGGAACAGTTGTTGATAGTGATTGGCAAGTTGGAGAACCATTATCGG
TTAAAGACTCGACTGGGAAGGTCATTATTACCGCAGACGTTGGTGCCGGTGGTAAGTTTACTTTCGA
TGTTCCTACTAAGTCCCAAGAACAAGTATTAACTCTGACTACTAATTTACCCAACATCGCTGATTCTCA
AATAACTATTAAGGCTGTGAGTTCATCGAATACTAACAAAGCTTTGCTAGAAAACTTGATCAACGCTG
CTGAACAGTTGGATAGTAATCAATATACTGTCAAGTCGTGGGAAGAATTACAAACTAAACTAACTGAA
TCAAAATCGATTCTGAACAATGATAGTGCTACACAAGATCAAGTAGACGCTTCCGTTAATGCTCTACA
AATTGCCCTTAAGCAATTAGTTCCTGTATCAAATAGCGGAAATAATGGTCAAAGCTCTAATGATAGCA
GTAACCAAAGTTCATCTAGCAGTAGTGGCAAAGAATCATCCAGCAATAGCAATGCCAATATTACTAGT
AAGGATCAGTCAGCTAAGGATTCAAATACGAGGCCTAAAGACCATAGTCTTTTGCCAAGTACAGGTG
AACGGGTGATGACGGGAATTTCTGTTCTAGGGGTAATTTTAATAGCTTGTGTGACTATATTATATATTC
GGAAAAAAGGACGCAGCTTTTAATTAGTCTCTGCGTCAACTGGCGTTAAAAACTAGATTGAAGTAATA
AAGTTACCACCTGGAAAGAGGCATGCTCATTGCTTGCAAGGGTGTCGACGTGTAATAGAAAAGTTGG
GG
SEQ ID 12 Position 325750 . . . 327159
TGGCGTGGGCAACGTGCACGTTTTCTAGTCGCTAAACTGTGAACAATGCTCGTGCTAAATGCAAAAC
TGAGCAAGGAGATGAACTATAAGCGGGGGACCCTTTGCTATTGAGGAGGAAGGCGAAGTAGAGAAA
GAGCGGTGATTTGAACTCGAAACAGCGGCGCCGCAGGCTAGCAGCACTGTTAGATTAATCGCCAGC
AGCAGATACTAGACAGCTTCTTAAAGGCTTGATAATAGCGTTGCGCCATTTCAATGGAACTAGTGGTC
AAAATCGCATTGTAGTTGCCATGACCCAAACTCGTTTTACGCGGGCCTTTTTGTAAAATATATTGAAC
AACTTGGTTAATATGTTCATCGGTCTCAAAGTCAGCCGGAGTTAAATACGTTTCTTCCAAGTCCTTCA
CTGACATCGCTTGAATCTTGGCTTGAATTTTCGCTTCATCAGCCGTACTCAGCAGGCGGCCTTGCTT
ATCACGACGAGCTTTAACGCGCTGAGTTTCTTTTTCAAGGGCCCGGGTTATTAAAGCGTCTTTACCAA
TCGTTGTCACGTGTTCCACATTAAATGGTAACACTGCTTGGTCCTCTAAGGCGTCTCGCAAGTTATAA
ACATGACAGACTTTACCAAATAGCTCCTCCGTCGTAACTGCTAGATCACCTTTGAGCTGTTTCTTATTT
TCATTAAAAATGGGGGTGCCAGTGTAACCATACCAGTTACTATTGATAAACGCTGCTCGAATTTCCTT
TTGCATCTTACCAAACTGCGACCGGTGGACTTCTTCAACAAAGAAGATCACCCGTTGCTTTAAAGTCT
TACTAAAGCGGGATTGCTTACCGGTTGCCAGCTGGACTTGCGTTTTTTTGACCGCCCGATGGAGCTT
TTGAATCGAGGTGACCAAGACCTTACCGTCATTTTGTTGCAATTTACGCATTAAATCACCGGTGTTTT
GGGCTTCGTTAATGGCAATATCATCATTGGCAGCATAGGCACTAAAGTTGCTGGTTGTCTGTTCGTCT
AAATCCCGCCGGTCAACTAAGAAGATGACCTTATCGACACCAGGATCTTGCGCAGCTAATTTAGCGG
TTTTATATGAGGTGAGTGTTTTACCAGAACCCGTGGTATGCCAAACGAAACCATCCTGATGGTCATGA
ATCCGGTGCATCACGGCTTCAATCGCATAAATCTGGTAAGGCCGTAAGAGAATTAAGCTTTGCCGCT
CTTGGTCGATGACTGTATATTCACTGACCATTTTGTGGGCCATGGGAATATTAAGGACTTGGCGCGT
GAACGCTAACCCGTTTTCCACGGGGTGATTATCCCGCGTCCGCCAATTGAACAAAAAGGCTTTATTG
AAATGATCCGGTTCGGCATTCGCAAAATACGCCGTACTATCCGGCGTCATAATCACAAAC
SEQ ID 13 Position 328723 . . . 329314
CGATCTAAAAGCTAAGTTATTTTCCAAGATATCAAACAACTTCTTAACCCAAGAATCTTCCACACATAG
GACAATAATGAATCCAAATAGATTCAGCTTTTTCCTGCAAACCGGGATCAGTATAAACGTCCAGTACC
GGATAATCACGCATTAAGTTCAGCTGCCAATGGGTATCATCTAAATTAAAAAGATCCGATTTAGTGTC
TCCCCTTACTACATTATGGCAATACACACAACTGTTGTTATACATGCTTCCTTGCTTTTTGATTTTAAA
CTCCTCCATTTTGCATATTATAAGAAGATTACTTCTACTTGATATATAGATGCTTTCCTTGCGAGGGTA
AGTCAGACAAGGAAGCATTTCTAACTTGAGATACTTAAGCTTGTCTCAATAGATGTAGATAGCGGCTC
CCCAATCGGATATTAACAGCTCAACTAGTCAAACCAGATATATAAATGTGACACAAGCTGGAATATAT
ATCATTATCTAGATAATTCAAATTGAGCTAATAAAATCAATAAAGAAAATTTTAAATAACATTATTTTATA
AACCCCTTTAGGATTTTCCCGATTTGATATTCTACGTATGTT
SEQ ID 14 Position 2002858 . . . 2005090
GAGTATCCAAAAATACGACGGGTATTTGAATAGGATACTTATTAAGCGAGAATGGTATTGGAAATCTG
TGGCAGCCACTCAGCGGAACCATACCTTTATCCCAACCCCACGCAAAAAAAACATCAAGTAATCCGT
CAGATATGATGACTTAATTGTGGGACAGTTCTAATATGAAGAAAACAGGTTAGATAATTGGGGTGAAA
AGATGGCAACCTATTCGCAGATAGAACTAGACATAATCAAATCATTTAAAGGGCTGATGAAAGACCAT
GAATTCACTGAGATCTCAATTAAAATGATCGCTGAAAAAGCCGATATCACTCGACGCGGCTTTTACAA
TCACTTCTTAGATAAATATGATCTTGTCAGTACCATCTTTGAGCATGATCTTTTTCCAACAGTCATCAG
TTTGACGAATATCAATGACTGGGATCAAGGGTCGCTGTTTATCGTGAATTATCTCCAAGACAATCGCG
ACTACTATAAAAAATTGTTGTCGCTTGAAGGACAAAACTGTTTACAGACAGACTTTTATAAATTGACTG
AGATGCAGATTGGGATCTTGATCCCAGAAATATTGGTCGGTAGGAAAATTTCTGACGAAGATCAGGC
ATTTTTAAGCGATTATTATTTTCACGCTTATATGGGACTGACTACCGAATGGGTCAAAGGTAAATATG
GTTTTTCAACTCAGGAGTTCGTTAAACGGTGGAAAGCCTTACTCAATAATTCAATGCATAATTATCTG
GACAACTACGCTCGATGAATTACACAGATTGGATTAAATGAGAAAGATGTTACATTTGTGCCAATATG
TGAATTGATAAATATTTCACAAGGAACTATTCTTTCCCTGTAAACGAAAGTTGACTTGAAAGGAGTTAG
TTCTGATGAGTAAATATAAAGTTATTATTTGGGGATTAGGAAACGTTGGTCGTTCCGCAGTGAGAATG
ATCGCGGAAAGACAAAATATTTTTGAATTGGTTGCAGCCGTTGACGTTGATCCAAAGAAGTTAGGTAA
GGATGCCGGAGAAGTCTTTGATTTTGACAAAGTCGGCGTCAAAGTTTCAGATGATATTGATGCAGCC
TTGAAACTTCCAGCTGACATTGTGCTCGACTTTTGCCCAACGGAAATGGACAAACAAGGAACATTCAT
GCCTTCTGCTATTCGACTCGCCAAATCGCTCGATGCCGGTAAAAACGTTATTACCACGATTCCGGTA
TATCATGTTCAAGACAGTCAGCCAGAAGTATATGAATATCTAAATGAACATGCTAAAGCACATAATGTT
GCTTTTGTACCATTTGGACTTTTGCCAGGCGATTATGCCTCATATATCCCACTAGTTTTGGCCGGGGC
CATGGGCCACGTGGATAAAATTGTTGTTCAATCCGGTGAAGATGACTGGCACAACACATCAGGCTGG
GTCGATGTCTTCTCATATGGCGGCGATATCAATAAATATCCAAAACCAGACTCAGACGAAGATCTCTT
GGCTAAGTTCATTTATGCTTATTATTCATCCGGCGTATACGAGATGGCCGATAGGATOGGTCTGAAAT
ATGATACCTTCAAACCAGAGCATGAAGTCTTCACTGCACCCAAAGATTTGGAAACGATCAAGGGTAC
AGTCAAAAAGGGCAGCATTTATGCCCACAGATTTACCATGGCACTTTACAACGGCAACGAACAAGTA
GCCGCCTTAAGATATGTTCATAAAGTTGATAATAAAGAGACACCAGAATTACCGATCAATAATACGAT
TCATATTGAAGGCTTGCCGTCAGTCGATGCGCAGATCGATGGATTGATCCCAGAAAGAGAAGGCTAC
GTTTCATCAGCCGCTCCAGCAGTCAACTTGATCCCTAGCATTCTCGAGACCGACAAGACAGGTTATG
TTGAAGTCTGCGACCTTCCAGTAGTGATTGCCAGGCCATTGGATATTGGCGCAAAAAAATTAGTCTA
GACTAGGCTTTCGAAGCTGCTTTGACCATTAAGGTTGGAGTAGCTTTTTCATTTGCAAGTAAATCATT
ACGGCTTGTGTATACGGTATACAAAATGGAGAAAACGCTGACTAGTTTATAAATCATTGAGACTTAAC
GGCCGGATAAATGCTGATCTGATTATAGAAATAACAACAAAAAGGCCACGCTAAAAATCATATTAATT
ATAATCGGGAAATTTATTAATAATATTCAAGAAAAATAAAAACCGTGGGTACATTATTTAAAA
SEQ ID 15 Position 2262750 . . . 2268615
TTTGAAACTAAGACGAAAGCTGCCATGTCAAACAAAGCCGCCATAAATGCCACTGTCACA
GATCCATCAGCCGCAATGCCAGCATCTTGCTGAAGTTCTTTAACAGCATTAAGGGTGTTA
TTCGTGAACTCATTATTAAAGTCTACTGGACTAATCCCTTTGCACCAGAAAGCCCCTTGA
ATGAGTTGAGCAATGTTCCCCTTATATCCTGGCTTCAGACTACCTACAACAGGTGCTAAG
GCGTTTTTGGTCGTCTCGCCAAAGCCTTCACCAATAGCACTAATACCGATTTCGTGCTGT
AATCCCATTCTTAGGCTATAAATTGTTGGCCATCCCGTTTGCCCGTTTTCTGGAGCTGCG
ACAAAGCCAGGAACGCTACCATACGTTTTGTTGAGCCATTTTTGAACGGCTCGTACTGCT
TCATCTGCCATTTTAAAGTCTCCTTTTTTGTTTTAGACAGCACGTCTGCCGTCACAAAAA
GCAAACATATGTTCGGATTCATTTCATCTCTTCAAAGCTTCGAAAGGCAACCCTGATCCA
CAAATAATCCTTTTATTTTGAACTTAGCAAAAAAATGAGGCCCTCACATAGTGTTGAAGT
TGCCTCATTCTTAATGTCTATATTTAAAGTATTGCCACAACGATGGATCATCGAACGCTC
ATGGACTTGGTTAGACATGTATCGACGACTATGAAAATGTGGGCGCAAGCTCAATTTCAC
CTTCCAAATGTTTGTGCTATCTCATTTAGCGCTGGTTTTTTTAGGATAGACTAGACAAGG
ACTAATAATTTCTCAAGAATCCCGCAACTCCACTATTCATTCGTCGAAATCCCCACTGAT
ACTCTTGTCCTTGCACGTTCGACCAAGCAAGAATGTTTATTCCGATAACCGAATTGTTGC
CATCAAGTAATGGACCTCCCGACATGCCATGATACGAATTAATTTGTTGTGAAATATATA
TTCCTAGTGGGTCCTCTGAAAAGGGTGTGACAGTCCCACTTGATTGAACCATGACTCCTT
GAAGTTCGTACCCTGATTGCGGATCGCCAGGGAATCCAATGGATCTTGCTGCCATCGTAT
CAGCAGGGTTCGTATTTAAATTAAGACCCGCAGGCATACTACCAGACTTCATAGAGACAA
TTGCAGCCCCGTAATCATTCGAAGTAGCTGTTGAATTATTAATCCATGCCTGTGGCACTA
TCAATCTATTCAATACTCCGTAACCGACCCCTTGATGATTTGCTTGACTATCACCAAAGT
TAATAATTCCTCCAGAAATATAATGACCATCATATAACATGTGTGCTGCTGTCCCTATAC
GGTCTACTCCAATGCTAAATCCAGTACCGCCAGAAGTTCCACTGCTCAGCTCTGTGCCAT
TTGAGTTAGAGATGATTTTTTTATACGAACTGTCAAGGTATGGTGAGTTAATGACCATGA
CACCATTTGCCATCGAAAACCACGTGCTCAAAACCCCAACGGAACTGTACGGCGCCGAAT
TGGGGTTTGACACAGGTGATACCGTCCTCACAGATAGATGATTAGACGATTTATTCAGTT
TCGCAAGATATTCGGACGTAATTCCTTGAACTTTCTTTCCTTCTTTTAAATCCTGATATT
GATCAGGTGTATAAGCTTTGACACTCCCTTTAAAATCGGGATAAGAATACTGATATTGTC
GAATGATTTCATTCAAAAACTGTTGTGTTGTCGTCTGGTTAGTCAAAACAATATCATTCT
TTGGTAAAACATGGCTATTCGCTGCCAAAGGATTTGCCATACTATCAGCACTTACACTTA
CCGTTTGAACTTGAATTATCGCTAAGGCTGCAGCTATCATAGTGATATATGCCCATACTT
TTCGCAATTTAATTCCCCCTTTTTCTTAAAATGAAACCGCATTCACGGAGGCTTGTCAAT
GCTTTTAAAAAACAAACGTTACTTTTGGCTCATCTTGGCTGTCAGCATAATTGGAGTAAT
TGTTCTTGCGGTGTTATGGCGCATGAACCCTGAAGGAACGGCCTCAAATAAGTTTGAACG
TCCCACCATTACTATTAAAAAAGTCAAACTTATTAAGCACAGTAACAGTATTGCTGTCAC
ATTTGCTACCTCTCCAAAAAGCAAATATACGATAAGTGATCTTAAAGAGAATCAACTTTC
TTCTGGCATTTCGAATAAAAGAGAAAATACCGTTTCGGAATTAAAGCCCTCCTCCTCTAA
GCTTGCAATACGGGTTAAGCATAACAATAATATACAAACCAAAGTGGTTTCTGTTCCCAT
TGGTTATCATATTATGAAAAGTGCCATTTCAAGAAAGCCAATTCCTATGGGAGAAGAGTT
TAAGTACAATGGAAAGTCGCATGTTTTATTTAGCATGACCATTACCCCTAAAAAACAAAA
CAAGAACAGTATAAAAAACACCACTGCTTTTAACATAACGGTTAAAAATGATCACTACCT
TGTCCCTGTCGTATTAGATACCAAATACCTTACTGTTTCTGATTCAGAAGGTAACTCATT
AAAAGTAAAGCCATTCTCAAAAATTTCTATTCCAGCAAAAAAGAAAAAGACCATTGCAAT
AACTATTGAGGGCGTTCCCGCAAGCTCTGCCAATGGTCTAGTTATAACGTATAATACTGT
CGATTTAGACTTACCAATCTCCTTTATAAATTCCTGAAATTACACTAACTGTCCCCCACC
TTGACAGTCAGTACACTCAAACTGTCTCTTATGCTTACAAACACGTAATTTAGGCGGTTT
TTAAGCAAAAGTCGTTAGTTTTCATAAATGTTATCTTATACTCTAATGAGATCTAGCTTG
TGATAATAAGGCTGTTTTTCTTTGACAGCCTTATTAAGCACACTAATCAATGTCAATTCG
AAGTTTTTGGTTTCCTACTTGGCCAACTTTGTTATCAGAAATTCCAAAACTCATTGCCTC
CCGCCACCATATATTTATCGAGCCATTTTGAAAATGAAAAATCGAAATATCGGTCTGCTT
CTATTCCGGGATGAGTTAGATATGATTTTCCTAACCGATACTTCTCTATATCAATATACA
TATCTCCGACATCCCTTATATGGAGAATGGGTACTTTATTTACAAAACTTTTAGGCAGTG
CCTCTTTTTCTTTAATCATTTTTCTAATTGAATAGACTTCACACGTATATCCCATAGGAA
TCCCTTCGATTGTTGTGTCAAATAAAAATAGTCCATTAGTAATCGAGAGAAACTCAATGT
AATCCTGTGGAAGGTTCCACCTTTTTATTTTTTCTATATCATCAGCGTGTGCAGGAGGTT
CTATCTTAAAAGAAACATTTTGCACATCTCCATCTAATTGGAATGTTGAGAGCGCTTTTT
CCCCATTTTTCGTCACCTTTATTAAAGAATTAATTCTTCGCCGAATTAGAGATTCCAAAT
GAGTTCCTCCTCAATAGTTGTTAAACCACGCGGTGATCAACCGATGATTTGGTGTTAACA
CCGGCATCAAATTATTAAAGTCATTTGTTCCGCCATAAACTCTCGGGCGAATATGATGCA
CTTCTCGAGAACTCCAAAAATCTGCAGATTGATTGCCATATGTTTCACTGAACGTTTTAA
TATAAATATATCTATCTTTTGATGACCAACTAGGAGATTTTGATAACTTAGTCCAGGTAG
TATTAATAGGTGTTTCTGCATCTTTTTTAGACACGGGATCAACATATTCAGGGAAATTCT
GTCCTATTTTATTTTGTAAATAGATGGCCGTTGGTGGGATTGGTTTTACATTAGCCGCTC
CATTAACTCCTATAAATCCAACAGCGCCAGCAACGTTAAAAAATGTGGTTTTGGCTGGCA
ATTCTGTAAAACCAAGTAATCCTGTTTGCAAACCGCCTATTTTGCTAATGGGTGCCAGCG
TATTGGCATTCACCGGACCTGCAAGGGTAGGCTCAGTATTAAATTCAATTTGCACATCAA
CTGCGGCTGGCGGGACACCCTCAATAGAATCAATCCAGAAATTGACTCTGAATTTTCCGG
ATATGTATTCTTCCGATAAGTGCCAGGTAATATTTGTAACAGGAACTTCTGCACGAGAAC
TTATGCCATTGCCATGACTATTATTCAGTACCACTTGTTTTCCAGAAGCGATAATTGGTT
GTCCATTTTCTGGATTACTTCTTGATAACTTGTCAGCATTGGTCAGTTGAGTATTGTTAT
CGCTAATACGGCTTTCAGTTGAAACAATTTGATTTAAAGATTGTGTTGTGTCTGCGGATA
CAATTGTTGAATACCCAAGCAAATTGACTAGAAAGAGCCCGAATATTAGCCCAATAACTT
TCCACTTTTTCACGCCTATTATCTTCTTTCCAAAGTTCTTCAGTGCCTGGCAATAACTGT
ATACATTGAGCAGTATAGTCGCTATTTTATAGCTGAACAACTCATAAAGCTCAATTATTA
TTAGCCTATAAAACCACTGCCTAAGTGAATTGATCTAGAACGAAGCACGCCGAAGAAGTC
GCTAAATGTGCTAAGAAAAATGTGCTTGAATAGCTCAAAAGTAATTAGCGTCTCCATTGA
AAATCCGTTATTTTTAAGTGATCTAGTGTTAACTATGAATCCCAAATAAAAAGCAAAATC
CGTAAATGCCAAATTTTCCTTTTTGACGTTTTTCTACTGTCGCGAGATTTGCAAGTGTAC
GTACACTTACGATGAATTGACAGAATCTCAGCTGCGCTGATCGTCAATTTTGTTTGGGGG
CACGCCCCCAATCCCCCTGTTATTTTGAAGGGAGGTGAGTCCCCCTTCAAAATCAAAATT
TAAACAGCATCTGCCGCCATCTTTTCGCTGACCTTCTCACGATGTTACACGTGGTGTTGA
CACCCACTTGCATTTAGAGTTTCATTCAAGTTGAACATTGTGTAATATATGAGTTGCATT
TGATAAACATATCAGTTGCTATTTGTGCAACTTTAAAGCTTCGGCTAATTCAACGTTCTG
TTAATTTACAAGCATCTCGACAGTTTCTGTTAAAGCAACATCTACGCTTCAATTCGAGCA
ACTCACTATACGTATGCCGAGTTGCAGACAAGCTACTATATAGCTGTACGCGCTGAAACA
CCAAAAATCGTTCGTTTATGCCCAATAAGCGAATAATCTTGCTCAGGTGTAGTAAAAAAC
TGTTTACGTGTAGTGAATGGCGCTAGCCCTTGTCGTAACTGGCATCATCCACGTGTAGTA
AAACGCGTTTTACTACACGTTCGTAATTTTTTCACGTGGAGTAAATGGCGTTTTACTACA
CCTTTTGACCCCAACGTGCTATCACGACAAACCAAACCGCACTGCGGTTTACCCCAATTT
TGGGGTCAGTTTTGCCTTATGCTCTTTCATGATTTTAGGCGCGTTCCAAGCAGTCTCAAA
AAGTGGTCGATCCAGGCGAGCCGATTTTTGAGAAGGATTGGATAGCAACTCAATTTATTT
TGATCTTTTGCTTGGAGAAAAACGTTCACGTTTTGACCAGGGCCGTCGCAACTGTTGACC
AAAACTCGTCCGGTAACGTGACGCTATTTAAACGCCGCGTTGGTTTGCTAGACGACCATT
CATCATCACCATTCAGGAGGTTTTTGAAATGACAAAGCAAGACGAAACACACCGGGTCAT
GTTCACTTTGACCGATCAGGCGATTGCAAAATTGAATCAGCTGGTCGCAAAAAAGCAACA
GGAAGTGAATCAAAATCCGGAACTGGCTAAGTACCATGTCAGCGTGACCAAATCAAATAT
CATTGAGGACTGGTTATCAAAGCAGTGAGTTTAAAAAGCGCTAAAGGGCCTGTACTAGCG
TTTCTTACTCTGGTGGGTATAATTAATGCTCTCTACATCAAAAACG
SEQ ID 16 Position 2776965 . . . 278971
GCCACGAACCTGTAGCCGTTTGGATGAAGCCATATAATACTGGACCAACCGCCGCAAATA
AGTAGCCGACACTTTGAGCAAAATCAGGAATACTAATCTACTTTGCCCTTAAAAAATCTT
GAGATGATCCATATCTTGTTTTGCCTTCATTACTGTAGTTGGTCATAAGAAGTGCCCTAC
ATTCATTAGATTACTTGTCTAATAATTGTAGGGCACTTGGGTTGAGAAAAATGATGTTAA
CTAAGAATGCAAACGAACTAAAATCTTTGCTTGCTTTTTATCCTTTTCTAAGGATTCAAT
TCCTTCTGAAACTAATTCATTTAATTCAATCTTTTTTGTAATGACCTGTTTGAATAGTGA
ACGATGGGTATCTATAATCTTAATTACTCGATCGAAGATATTGGCATATCCATAAGATGT
TAATAAACTACCACCTTTTTTAAGAAGAGCTCTAACATCTACAACTGGTGGATGTTGAAA
TAATGCAATCACGGTAACCTTGCCACCATTTTTAAGAGCCTGAATGGCACCAGTAAGTGT
GGGTTGTACACCGGCGCAATCAAACGCAATATCCACTCCCTGATTTTCCGTGATAGTGCT
GATAGCGTGAGCTAATGACTTTTGACTATCAGCACGTATTGGGTATTGAATTCCTAATTC
ATTTGCTAAATTCAAACGTTCCTCTGACATGTCATTTATTATGACGTGATGTGCACCAGA
AATTTGTGCTATTAAGGCCGTGAACAATCCAATTGGACCAGCACCTTGAATTAAAACATC
ATCTCCAGGAGACACTCGGCTTGCCATAACTGCCTGTGCAGCAACTGAAACTGGTTCAAC
TAAGGCCCCTAAATCAAGCGGAAAGCTAGCTGGTAAGAGATGTGCAAAGGTACTTTTTAC
ATTGCACTTTTCAGCTAAGCCACCGTTAGCCGAAAATCCTAAGAATCCTGCTGATTGATC
ACTACCTATAGCATGTTCACACCAATTATAATGACCAGAAAGACATTCCGGACATTTTCC
ACAAGCAATCATTGGTTCGACTGCAACTTTATCTCCAATTCTTAATTTAGATACTTGTTT
TCCAATTTTAGAAATCGTCCCAGAAAATTCATGACCAGGAATTAGCGGGGCTTGCATATG
GGTTAGCGGATGAGGTATTGTCGCCAAATCCATACCCTCTAAATATTCATGAATGTCACT
ACCGCAAATACCATTAAATGCAACCTCAATTTGAACTTCATCTGGTGCGGGATCAGGAAT
ATTTCTTTTTTCAAAGCGGATATCCTTAGGACCGTAAATAACAGCTGCCTTCACCATAGT
CATAGTGCTTCGCTTCCTTCATGTTCAATATAGCACAATCGTATATAAAATAGTGAATAG
ATTTCAGTAATGAAGTTACCATCTTGACTTAACAAAAACTTGCTAACTGATTATATGAGA
AACTTTTACTTGAAACATTTTTGGTGATTACCATTAATTCCCTCGGACATATTTTGAAAA
ACCCTATTTGATGCTGATTGCAAATTATTTTATGCGTATTTATTAAGGGTTTCTATGTTG
AAGTATATAGCAAACTTGTTCAAGTAACTGACTTTCACGTGGGCTTTAGCCAAGAGATGC
TGAGCAGCGAACCCAAGGGGTGTTACTCGCCCACGCAAAAAAGAAATCCAATTGCATTCC
AGTATGAGCGAGAAGCAAGCCATTAAGACGCTGATTCATGAACTCGCGCACAGTGAATTA
CATTGTGATCCGAAGTTAAAATTGGATCGTTCAACCATGGAATTGGAAGCTGAAAGTACC
GCGTTTATCGTTTGTCAACATTTGGGAATTGACACGAGTGATTATACGTTTCCTTACCTT
GCTGTTTGGTCGAAAGATAAGGATCTTTCCCAGCTCTCCAAAAGCTTAACGCGTATCCAA
TCCACCGTCGAAAAATTCAATAAAACCGTCGATCAAAACCTTGAAAAGATTCGTGAGAAA
CCGTTGACGCTTGATCAAAAAATAGAACGCGCTAAAACCATTGCGACAACGGAAAACATC
GCAAAAAAAGAGCAAGGGCTGGTGCAAGCAACGCAGGAGAAAACACGCTAACCCATTTGT
TGAATACTCTCACTCAAGAGGACACTCCAGCCCTTGATCACCCAAGAAAGGAATTACCAA
CATGAAAACCATTGACGAAATGAACGAATTCGATCGTGACATTATCTTACTTCACCGCAA
GTCTGTGAGCGAAGATACACCGCAGGCAATTCTTGTGAAAGTGAAACAGATTCGTAACGC
AATTGCCGACGAAAAGGGGGGTAAAGAAGATCCAATTGAGAAAGAATTTACACTCGAATG
TTACGACGAAGCAATCAGAAAACTAAGGGACCTTTCGGTCGCTGATTATCAGTTGTGGTT
GCGTCAAAACAAAGACCTGGAAGGGTTTGAATTTTGATTTTGAAGGGTGTCGTAGACCCC
TTCAAAATAACGGGGGATTGGGGGCGTGCCCCCAAAACAAAATTGACTATCAGCGGAGCT
GATATTGGATCAATTTATCGCAAGTGGACGTCCACTTGCAAATCTCGCGACAGTAGAAAA
AGCCCCAGAAAAGCAAATCTGAAAAAATGTAACAGGCACTTGATATCAAGTGCCTTATTG
TTTCTAGGATCGCTAAAAATAACAGGAGGTGGTTACATGAAGCAATCTGATGAACACCGC
ACGCGTTCAGTGAGAAGCACTGTGCGTATGACCCCAGAGGAGCGTGCTTGGGTTGATATG
AGAAGAGCCTCTGTCGGCAATCCAAAGTTCAATGCATTTGCCTGTCGCGCACTCACGACG
AGCAAGATCGTTCATGTACATTTTACTGATACTAAAAAGTTACTTAGACAGCTGTCAAGA
ATTGGGAATAAGGCTCCTATGCTGTAATTACGGACAAAAATAGTTTGTGCGATAATTACA
GCATAAGGGCCTCTAGGTCGGAGCCCAGGAGGCGGAGACCGCCGCACAGCCCAACCCCAC
GCCGAACCGGAGGCCAGCCCGCCCGCACCGCGGCCGCAATCATCCACCCAACGCCCCCCA
AGTTTTTGATAGCGGTAACAACGCCTGTGCGCTTGTCGTGGCCGGCCTTTTTTCATAAGG
TTGGAGGAGAAAGGAAGGGTGGTTATGGGCGCTTGGTATGAACACGCAATTATTTACCAA
ATCTATCCAAAATCGTTTCAAGACAGCAACGGCGACGGCATCGGGGACCTGAACGGGATC
CGGCAACGGATCCCGTACCTGCAAGCCCTCGGCATCAACACGGTGTGGCTGAACCCGATC
TTCGTCTCCCCACAGGTGGATAACGGCTACGATGTTGCCAATTACTTCGCCGTGGACGAA
ACCATGGGTACGATGGCCGACCTGGAGGCGCTGATCGCGGCTCTGCACGCGGCCGGCATC
CGTCTGATCATGGACTTTGTGCTAAACCACACCTCGGATCAGCACCCGTGGTTCCAGGAC
GCCATTCACGCCAAAAATAGTCTGTACCGCGACTACTACATTTTCTCTGGCCACGACGGG
CAGCTGCCAAACAACTGGGGCAGCTTCTTCGGCGGATCGGTTTGGGCGCCGGATCCGGCG
GGAACCGGGCAGTCGTATTTTCATCTGTTCGACCGGCGGATGCCGGATCTGAACTGGGCC
AATCCCGAGGTGCGGCGGGCGATGGGAGACGTCGCCACGTTCTGGCTCGGCAAGGGCATC
GACGGACTGCGGCTGGATGCCTTCATCCACATTGCCAAGGCCGATCTGGGGCAGGATTAC
CCCCTGGCTCCGGGGCAGCAGACGCCGGTGGTGGCGGAGCCGTTTTTCTCCAACCTCCCG
AAGGTGCAGGAATGGCTGCGGCCGTTCTGCGACCGGATCAAAACCGACTACCCCGACGCG
TTTCTGCTCGGCGAGGCGGCATCGGCCAACGTTAACCTGGCGGCGGATTACACCGCGCCT
AGCCAGCACCTGATGGACAGCGTGATCACGTTCCGCTACTTCACCGAGGACGAAAGCGGC
CTGGATCCGCGGCTGCCGGCGCAGTACCAGCCGCGGACGCTGGATTTCCCGGCGTTCAAG
CAAACCCAGGCGGTGTGGCAGCAGACCCTCGCCGGGGTGTCGATGCCGACGCTGTACTGG
GGCAACCACGACATGGCCCGGCTGGCGACGCGGGTGGCCAAAACCACCACCCAGGCGCGC
AGTCTGGCCATGCTGATGTACCTGCAGCGCGGCCTGCCGGTGATCTACTATGGCGAGGAG
CTCGGGCTACACAACCTGCAGTTCGATCACGTTGATCAGTTTGCGGACGTTTCGGTGGCG
CCGTTCGTGGCCGCGGTCGAGGCCACCGGGCAGTCGCGGAGCGCGGCGCTGGCCATGGTG
TCGGCGACGCACAAACTGCCGGCACGGGGGCCGATGCCTTGGACGACCGGGTTGCACCAG
GGCTTTTCCAATCACCTGCCGTGGCTGGTTGGGCGCAGCGAGGACGTGACCAGCGTGGCC
GCGCAGCAGGCCGATGAGGCCAGCATGCTGCACTTCTACCAAGCGCTGATTGCCCTGAAG
AAGCAGCCGCTGTTTCAGGCCGGGCATTACCGGCTGCTGACGACGGCGCCGAACCTGTAC
GTCTACGAACGCACGCTGGCCAGCCGGGGGGCCCTGGTGGCGGTGGCCTTGGATGAGCAA
GGCGCCACCTTCACCGTGCCTGAAGGCCTGACGACCGTGGCGCTGGCCGCCGGCGATTAC
CAACTCGAAGGTCAAACGCTCACGCTTGGCGCGAACGCCGGCGTGGTGTTAAACGAAAGG
GGAACTCGATAACCATGCAACTTGCAGCATTACGGCACCGCCCAGAAAGCGAAGATTGTT
TTTTGTACACTCCAGATGAGCTGCGGCTGCGGCTCCACACAGCCAAGGCCGACGTGCAGG
CGGTCATCGTACTGTACGGGGATCCGTATGTCACCGCGCCGAACCCGACCACCGGAGAAC
CGGAATTCGCCTACCAAGAGGCGGCGATGATCAAAACCGGCACCGGCCAAACCAGCGACT
ACTGGACCATCAGCCTGACCGCGCCTTATCACCGCCTGCAGTACCAGTTCCTGGTGACCG
GTCAGGACGGCAACACCGTCCTGCTCGGCGACCGCGGCTTGCGGGCCGACAGCGCCGCCA
ACCGCCGGGCCGATCTGTTCCGGGTGCCGTACTTCCACGCCATCGACACGGTACAGACGC
CGGCCTGGGTCAAGGAAACCGTGTGGTACCAGATATTCCCGGAACGCTTCGCCAACGGGG
ACAAGACGAACGACCCCAAGGGCACCAAGCCTTGGCGTCCGGOGGATCACCCGGGCCGTG
AGGATTACTACGGTGGCGACTTGCAAGGGGTGCTGGACCACCTGGACGACCTGCAGGCGC
TCGGCGTGAACGGGCTGTACTTCTGCCCGGTGTTCACGGCGATGTCGAATCACAAGTACG
ACACCATCGACTACTTCAACATCGACCCTGCGTTTGGCGACAAGGCCTTGTTCGCCGATC
TGGTCAACCAAGCGCACCGCCGCGGCATGCGGGTGATGCTGGACGCTGTGTTCAACCACA
TGGGCAGCCGCAGCATGCAGTGGCAAGACGTGCTGAAGTTCGGTCCGCAGTCGCGCTTCG
CCTCCTGGTTCCACATCAACCGTTTTCCGGCGGCGCCCTTCGCCGCGCCGGAACAGGGCG
GCGTGCCGCAGTACGACACCTTCGCCTTCGAACCGCACATGCCGAAGCTCGACACCAGCA
ACCCGGCGGTGCAGGACTACCTGCTTGAGGTGGCGACGTACTGGATCAAACAGTTCGACA
TCGACGCCTGGCGGCTGGATGTGGCCAACGAGGTGGACCATCACTTCTGGAAACGGTTCA
ATCAGGCAACCAAAGCGCTCAAGCCCGATTTCTTCGTGCTGGGCGAGGTCTGGCACTCCA
GCCAGCCGTGGCTTAACGGGGATGAGTTCGATGGGGTCATGAACTACGCGTTCACCGAGC
AGATCGAGGCCCACTTCCTGACCGGCAAGCTGAGTGCTCCTGAGCTGACGGCGGCGCTGA
CGGATCAGCTGATGCTGTACCGCGACCAAACCGACCAGGCGATGCTGAACATGCTGGACT
CGCATGACACCGCGCGGCTGCTAACGGTGGCCGGCGGCGACGAGGACCTGGCCCTGCAGG
CGCTGGCCTTCACCTTCCTGCAAACCGGGATGCCGTGCCTGTACTATGGCACGGAAATGG
GCATGGCCGGAGAAAACGATCCCGACTGCCGGCGGCCAATGGACTGGGCCCAGCTGAAGG
GCCCGATTTGGCAGCGTGTGCAGGCCCTTGTGACCTTCCGCCGCGCCCAGTCGGCAACGC
TAGGCACCGGCACCACGGCGCTGAGCGTGACCGCAGCCGGGCTGCTTAAGGTAACCCGCA
CAGGTGAGCACACCGTGACGGCGTATTTTAACACCACCAAGCAGATGGCGACACTGACCG
TCAGTCCATTACTGGCGCAGGGTTACGCCGGCCAGCGGCTGGCGCCAACCGGGTTTGCTG
TTATGGTTCAGTAAGATTATGTTAGCGGTAACAGGCAATTTGACCTTTTAAAAGCGTTTT
CATATTATCATAATCAAAAGTGTAGAAAAGTTCAGGTGGCGCAATTCACCTCCCGAAAGT
GAAGGATGCAAGATGAAACGGATATTTGAAATCGACCCGTGGCTGGTGCAAAGCCACCAA
TTGAACCCCAACGAGAAACGCCTGCAGGAAAGCATGACCGCCATCGGCAACGGCTACATG
GGTCTGCGCGGTAACTTCGAAGAAGGTTACAGCGGTGATCACCTGCAAGGCACGTACCTC
GGCGGCGTCTGGTTCCCAGATAAAACCGTCGTCGGTTGGTGGAAAAACGGCTACCCGGAT
TACTTCGGCAAGGCGATCAACGCGCCGAGCTTCATCGGCATGGCGCTCACCGTGAACGGC
GAGCGCGTCGATCTGGCCACCAGCGTCTACCGCGATTTCACCCTCACGCTTGACCTGCAC
CAGGGCCTGCTGACCCGGAGCTTCGTGTTCGAGGGCAAAAAGGCCACGGTGCGCTTCACC
TTCAAGCGTTTCCTCAGCAACGTAATCAAGGAGGCGGCGCTGGTGCAGCTCACCGCCGAA
AGCCTTGTCGGACCGGCCGAGCTGACGGTGGCCGCACAGCTCGACGGCAACGTCACGAAC
GAGGACAGCAACTACGACGAGCGCTTCTGGGCACCGCAGGGGGAAAACGCCGCGGCAGGC
ACCATCCAGCTGCAGACCAAGCCCAACCCGTTCGGGGTCCCGCAGTTCACGGTGCTGCTC
AAGCAAAGCCTGCGCCAAGGGGCAACCCTTTTACCCGGCACCGTGACCACCAGCACCGGC
CAGCTGACCAGCACGGTCACGCTGCCGCTGGCGCCAAACGTGCCGGTCCAGCTGGAAAAG
GACGTCATCGTGGTCACGAGCCGCGACGTCGCCCCTGAGGCCCAGGCCGAAGCGGCCGCG
GAGCTGATGACACAGCTGCAGGGCCAAAGCTTTGCGGCCCAGCTGGCGGCACACACCGCC
CTGTGGGCCAAGCGCTGGGCCCAAAGCGACGTGGTGATTGAAGGCGACGACGCGGCCCAG
CAGGGGATCCGCTTCAACCTCGCCCAGCTGTTCATGACCTATTACGGCGACGATAAGCGG
CTCAACGTGGGGCCGAAGGGTTTCACCGGCGAGAAGTACGGCGGGGCGACCTACTGGGAC
ACCGAGGCGTACGTGGTGCCGATGTACGTCGCCGCCACCCCTCCGGCCGTGACCCGGGCA
CTGCTGCAGTACCGGCACGACCAGCTGCCCGGCGCCTACCACAACGCCCAGCAGCAGGGG
CTCAAAGGGGCCTTGTTCCCGATGGTGACCTTCAACGGCATCGAGTGCCACAATGAATGG
GAAATCACCTTCGAGGAGCTGCACCGTAACGCAGCGGTCGCCTTCGCGATTTACCAGTAC
ACGGCCTACACCGGCGATGAAAGCTACGTCAACCACGACGGCATGGAGGTGCTGGTGGGC
ATCAGCCGCTTCTGGGCGGACCGGGTCCACTTCTCCAAGCGCGCCGGCAAGTACATGATC
CACGGCGTCACCGGGCCGAACGAGTACGAAAACAACGTCAACAACAACTGGTACACCAAC
ACGATGGCCGCCTGGTGCCTGGAGTACACGCTGGCCCGGCTGCCGAAGGCCGATGCCGCC
ATTCAGGCCAAGCTGGCCGTGAGCGCCGCCGAGCAGCGCCAGTGGCAGGACATTATCGAC
CACATGTACTATCCGGAGGACAAGAAGCTGGGCATCTTCGTCCAGCACGACACCTTCCTG
GATAAGGACCTGCGGCCGGCAAGCTCGATTCCGGCCGACCAGCGGCCAATCAACCAGCAC
TGGTCCTGGGACCGAATCCTGCGGTCGCCGTTCATCAAGCAGGCGGATGTGCTCCAGGGC
CTGTACTTCCTGAACAATCGCTTCACCCGCGAGCAGAAGGAACGCAATTTTGACTTCTAC
GAGCCGCTGACGGTGCACGAAAGCTCGCTGAGTGCCTCGATTCACGCGGTGCTGGCCGCC
GAGCTCGGTAAGCAGGATAAGGCCGTTGAACTCTATCAGCGTACGGCTCGTCTGGACCTG
GACAACTACAACAACGATACGGCAGACGGTCTGCACATCACCTCGATGACCGGCGGCTGG
CTGGCTATCGTGCAGGGCTTCGCCGGCATGCGCTACGACCACGATCAGCTGCGGTTCGAT
CCGTTCCTGCCGAAGCAGTGGCAGGGTTACCAGTTCCGCATCAACTACCGCGGCCGGGTG
ATCCAGGTCGCGGTGGGGAAAACCGTTGCAGTGACCCTGCTGGCCGGCCCGCCGCTGACC
GTCATGGTTGCCGGCCAGCCGCAGCATTTGGAGGTGAGCGCGCATGCTTAAAGGATTGCT
GTTCGACCTCGACGGCGTCTTGACCGACTCGGCCAAGTTCCACCTGCAGGCCTGGAGCCA
GCTGGCCACCCAGCTGGGCATCACCCTGACGCCGGCCGAGCGCGAAGGCCTGCGCGGCCG
CTCGCGGCTGGACTCGCTGAACCTGATTTTGGCGGCAGGCGCCCAGGAAGACCGGTTCAG
TGCCGCAGAGAAAACGGCGCTAACCGACCAGAAGAACGCGGTGTACCTGAAGCTGATTCA
GACGATGACGCCGGTGGACATCCTGCCGGGCATTCCGCAACTGCTGAAGGACGCGCAGGC
GGCCGGCCTGAAAATGGCAATCGCCTCGGCGTCGCGGAACGCCCCGACAATTCTTGACCA
CCTGGGCCTGGCCGCCAGTTTCGACGCCATCGTCGATCCGGCGACCCTGCACCGCGGCAA
GCCCGACCCGGAGATCTACCAGCAGGCGCAAGCGCTGCTGGGGCTCCAGGCCGCCGAGGT
GATCGGCTTCGAGGATGCCTCGGCCGGGGTCGCCGCCATCAAAGCGGCCGGTCAGTTCGC
GGTTGGCATCGGGGATGCCCGGCTTCTGGCCGCAGCGGATTACCTAGTGAAAGACACGGC
GGCCCTGCAGCTGAGCCAGTTGCAAGCGGCGTTCGCCAAAGAAAGTGGGGAGACTAATGG
TTGAAATCGACTTGGACCACCTCTACAAGAAGTACGACGACGGCGAGGATTACTCGGTGG
TGGACTTCGACCTTCACATCAAGGATAAGGAGTTCATCGTGTTCGTCGGCCCCTCGGGCT
GCGGGAAGTCCACCACGCTGCGTATGATTGCGGGGCTGGAGGACATTACCAAAGGCGAGC
TGAAAATCGACGATAAGGTGATGAACGACGTGGCCCCCAAGGACCGCAACATCGCCATGG
TGTTTCAGAACTACGCCTTGTACCCGCACATGTCAGTGTACGACAACATGGCGTTCGGCC
TAAAGCTACGGCACTACAAGAAGGAGGACATCGACAAACGCGTGCAAAACGCGGCGGAGA
TCCTCGGCCTGAAGCCGTTTCTCGACCGGAAGCCGGCCGCCTTGTCCGGGGGCCAGCGGC
AGCGGGTGGCCTTGGGCCGGGCCATCGTCCGCGACGCCCCAATTTTCCTGATGGATGAGC
CGTTGTCGAACCTGGACGCGAAGCTGCGGGTGTCCATGCGGGCGGAAATCGCCAAGCTCC
ACCAGCGCCTGAACACCACCACGATTTACGTGACCCACGACCAAACCGAGGCCATGACTA
TGGCCGACCGGGTTGTCGTCATGTCCGTTGGCCACGTGCAGCAGATTGGCACCCCGGCCG
AGATTTACCAGAACCCGCGGAACCAGTTCGTGGCCGGGTTCATCGGGTCGCCGGCGATGA
ACTTCTTCAACATGACCTACCAGGACGGCTTCGTCAGCGACGGCCAAAGCATTCGCCTCA
AAGTGCCGGAAGGCCGGGCGAAGATTCTGGACGACCAAGGGTACAACGGCAAGGAAGTCG
TGTTCGGCATCCGGCCGGAGGACATCCATTCGGAGGAGGCCTTCCTGGAGACCTGGCCGG
ACGCGGTTATCAGCTCAACCGTGTCGGTGTCAGAGCTCCTGGGCGCCACCGAGCAGCTTT
ACCTGAAGGCGGATGACACCGAGTACGTTGCCAACGTCAACGCGCGCGACTTCCACAATC
CCGGGGATCATGTGAAAATGGGCTTCGACGTCAACAAGGCGCACTTCTTCAACAAGGACA
CGACCATGGCCATCGTGGCTAAGCCGATTCCGCTGGAAGGCTGAGGAGGTGAGTGCATGA
CCCCATGGTGGCAGCAAGCCGTCATTTACCAGATCTACCCGAAGAGTTTTCAGGACAGCA
ACGGGGATGGCATCGGCGATTTGCCGGGGATTACCAGTCGCCTTGATTACCTTAAGCGGC
TGGGCGTCGATGCCCTTTGGCTGAGCCCAGTGTATGTGTCGCCCGGCGAGGACAACGGCT
ACGACATCGCGGACTACGAGGCCATCGATCCCCAGTTCGGGACGATGGCCGACATGGACG
CCTTGATCGCCGCCGCCAAGCAGCGCG
SEQ ID 17 Position 2793833 . . . 2794809
CCCGCGATTTTGGCGTGATTGGCTTCGACGGGGTATTCCTGGACCAGGTGTCCAACCCCA
AGCTGACCACGGTGAAGCAGCCCGTGCAGCGCCTCGGCGAACTGCTGGCCCGCATGCTCC
TGCAGAAGGTGGCACAGTCCGGCGCCCAACAGGGGGAGCTGCTGGTCGATCCTGAGCTGA
TTGCTCGGGACACGACGCGAAAGTAGATCGGATTTCAACTGTCCTTACCGCTATGGTAGG
GCCAGTTTTTAGGCTCTATGTCAAATCTAATTCATAGCTAATAGTTGATTTGGCAACGCC
TAAGGCGTCAGCCATATCTTGGTAAGTATGATGGCCTTCACTGACCAGTTGAGCTAGCGC
ACCACGTTGAAAACGTGATAAAGTAGAAGTACCCAAAGTAATCACTCCTTATAGCTGGTT
GGAATTAACTACTCCATTGTAAGAGATTGCTTTGGGCCTTTTTTTATTTTTGTTCGGATT
AATTATAGAATTTGTCTAATTAGTTGAAAATTCTTAGGGTTGCCCATATATCTTTTAGTC
TGGTCATTAGTTTTTATGTTTGATCTGCTTTTTTCTGATCGCAAACACCCACAACTGCGA
GTGAGTCCTTTTTGAAGTCGTTGACTGTCAACAATACATTTATTTCCACATTGACATTGA
CAGAGCCAAAGCGCGTTGCCATTAGAACTGOTTCCAAAAAAGCTAATAACTGTGAGTCGC
CCAAACGTTTGATTAGCTAAATCGATACGCTTTTGCATACTAATCCTCCCGCTTGATAAG
AAGGTACTTAAATAGTTGCTTTCAATTGATCTAATCGCCATTGGCACCATGAAATAAAGG
CTAATTCGTCAATCTTTGGAATGCCATAGGTTCTAGCATACGTTAACTTTTGAGTGGTGA
GTAGTTGATCATAGGGTTTACTAATAATACCAACCACAAGGATATCGACTTTTTTGTCAA
TCCCGTTGACAGGCTTT
SEQ ID 18 Position 2967081 . . . 2968319
AAATACGCAAAAGAACCCGACGAGAGTTAAGTCTCATCGGGTTCTCAGTCGTGGATGAAT
TAGAAGCATTGTTAGCTGCATAACCTTCAACATAGGATCAATAGCTGGTTAGATGGTCAT
CTCTCAGACTGTTTGCACCAGATCCAGGCAAACGTGTTTATATCCTTGGTCATACTCAGG
ATAGATGGGCATTGTGAGTGCAACAGGACTTAGTTGCTTGTATGCTAGGCAATGTTGGCC
TTGATACAGAGGATCACTCTGTTTCTGATCTGGATGATAACCTCGTTTTTCCATTTCAGG
ACGCTGTCCCAATAATTGTTGCCCTGGGAGTTTGTCGAATTAAGTCTTGGTGCCACAAAC
ACACGCTACGGCTTCTTTTCTTGCAAACTGATCTTAGTATTTAGGGCAGTTGCATGATTA
CGGAATTGAACCATTTTATAATGAATCGTCTTTTCTATAAGTCTATAGAAAGTGCAGGTA
ATGGCATTTTCTCCAGATCGGATTGTCTAATCAATTTAATTGATTTTTTTGGTGTGTTTG
ATTATATTGCTTTTGCAAAGGTACAATATACCTTTTCTCTGCTGCCTTGCGAGCAGCGAT
GGCATCCTCCATATGAACATATACACGATTTAGGACAAGATGGCCTTGAAAGTACAGCCT
TGCGACCCACTTTTGAGCAGTTTTATCCCAACTAACTCCGATAACGCCAGATTTGTTATT
GGACCGTTTAAGTGTAGAAGCAACTAAATTTGTTCGATTAATTAGTTGAAAATTCTTGGG
ATTGCCCATGTATTTTTTAGTCTGGTTATTAGCTTTTATGTTTGATCTGCTTATTTCTGA
TCGCAAACACCCACAACTGCGAGTGAGTCCTTTTTGAAGTCGTTGACTGTCAACGATACA
TTTATTTCCACATTGACATTGACAGAGCCAAAGCGCGTTTCCATTAGAACTGCTTCCAAA
AAAACTAATGACTGTGAGTCGTCCAAACGTTTGATTGGCCAAATCGATACGCTTTTGCAT
ACTAATCTCCCCGTTTGATAAGAAGGTACTTAAAGAGTTGTTTTCAATTGATCTAGTCGC
CATTGGCACCATGAAATAAAGGCTAATTCGTCAATCTTTGGAATGCCATAGGTTCTAGCA
TACGTTAACTTTTGAGTGGTAAGTAGTTGATCATAGGGTTTGCTAATAATACCAACCACA
AGGATATCGACATTTTTGTCAATCCCGTTGACAGGTTTT

### REFERENCES

Bjerkan, L., Schreurs, O., Engen, S.A., Jahnsen, F.L., Baekkevold, E.S., Blix, I.J., and Schenck, K. (2015). The short form of TSLP is constitutively translated in human keratinocytes and has characteristics of an antimicrobial peptide. Mucosal Immunol 8, 49-56.
Capone, K.A., Dowd, S.E., Stamatas, G.N., and Nikolovski, J. (2011). Diversity of the human skin microbiome early in life. J Invest Dermatol 131, 2026-2032.
Cho, I., and Blaser, M.J. (2012). The human microbiome: at the interface of health and disease. Nat Rev Genet 13, 260-270.
Dominguez-Bello, M.G., Costello, E.K., Contreras, M., Magris, M., Hidalgo, G., Fierer, N., and Knight, R. (2010). Delivery mode shapes the acquisition and structure of the initial microbiota across multiple body habitats in newborns. Proc Natl Acad Sci USA 107, 11971-11975.
Ehlers, S., Kaufmann, S.H., and Participants of the 99 Dahlem, C. (2010). Infection, inflammation, and chronic diseases: consequences of a modern lifestyle. Trends Immunol 31, 184-190.
Fornasa, G., Tsilingiri, K., Caprioli, F., Botti, F., Mapelli, M., Meller, S., Kislat, A., Homey, B., Di Sabatino, A., Sonzogni, A., et al. (2015). Dichotomy of short and long thymic stromal lymphopoietin isoforms in inflammatory disorders of the bowel and skin. The Journal of allergy and clinical immunology 136, 413-422.
Hanabuchi, S., Ito, T., Park, W.R., Watanabe, N., Shaw, J.L., Roman, E., Arima, K., Wang, Y.H., Voo, K.S., Cao, W., and Liu, Y.J. (2010). Thymic stromal lymphopoietin-activated plasmacytoid dendritic cells induce the generation of FOXP3+ regulatory T cells in human thymus. J Immunol 184, 2999-3007.
Jang, Y., Jeong, S.H., Park, Y.H., Bae, H.C., Lee, H., Ryu, W.I., Park, G.H., and Son, S.W. (2013). UVB induces HIF-1alpha-dependent TSLP expression via the JNK and ERK pathways. J Invest Dermatol 133, 2601-2608.
Jin, S.H., Choi, D., Chun, Y.J., and Noh, M. (2014). Keratinocyte-derived IL-24 plays a role in the positive feedback regulation of epidermal inflammation in response to environmental and endogenous toxic stressors. Toxicol Appl Pharmacol 280, 199-206.
Kong, H.H., Oh, J., Deming, C., Conlan, S., Grice, E.A., Beatson, M.A., Nomicos, E., Polley, E.C., Komarow, H.D., Program, N.C.S., et al. (2012). Temporal shifts in the skin microbiome associated with disease flares and treatment in children with atopic dermatitis. Genome Res 22, 850-859.
Naik, S., Bouladoux, N., Wilhelm, C., Molloy, M.J., Salcedo, R., Kastenmuller, W., Deming, C., Quinones, M., Koo, L., Conlan, S., et al. (2012). Compartmentalized control of skin immunity by resident commensals. Science 337, 1115-1119.
Noh, M., Yeo, H., Ko, J., Kim, H.K., and Lee, C.H. (2010). MAP17 is associated with the T-helper cell cytokine-induced down-regulation of filaggrin transcription in human keratinocytes. Exp Dermatol 19, 355-362.
Schaper, K., Rossbach, K., Kother, B., Stark, H., Kietzmann, M., Werfel, T., and Gutzmer, R. (2016). Stimulation of the histamine 4 receptor upregulates thymic stromal lymphopoietin (TSLP) in human and murine keratinocytes. Pharmacol Res 113, 209-215.
Siracusa, M.C., Saenz, S.A., Hill, D.A., Kim, B.S., Headley, M.B., Doering, T.A., Wherry, E.J., Jessup, H.K., Siegel, L.A., Kambayashi, T., et al. (2011). TSLP promotes interleukin-3-independent basophil haematopoiesis and type 2 inflammation. Nature 477, 229-233.
Volpe, E., Pattarini, L., Martinez-Cingolani, C., Meller, S., Donnadieu, M.H., Bogiatzi, S.I., Fernandez, M.I., Touzot, M., Bichet, J.C., Reyal, F., et al. (2014). Thymic stromal lymphopoietin links keratinocytes and dendritic cell-derived IL-23 in patients with psoriasis. J Allergy Clin Immunol 134, 373-381.
Watanabe, N., Wang, Y.H., Lee, H.K., Ito, T., Cao, W., and Liu, Y.J. (2005). Hassall's corpuscles instruct dendritic cells to induce CD4+CD25+ regulatory T cells in human thymus. Nature 436, 1181-1185.
Wilson, S.R., The, L., Batia, L.M., Beattie, K., Katibah, G.E., McClain, S.P., Pellegrino, M., Estandian, D.M., and Bautista, D.M. (2013). The epithelial cell-derived atopic dermatitis cytokine TSLP activates neurons to induce itch. Cell 155, 285-295.
Yockey, L.J., Demehri, S., Turkoz, M., Turkoz, A., Ahern, P.P., Jassim, O., Manivasagam, S., Kearney, J.F., Gordon, J.I., and Kopan, R. (2013). The absence of a microbiota enhances TSLP expression in mice with defective skin barrier but does not affect the severity of their allergic inflammation. J Invest Dermatol 133, 2714-2721.
Ziegler, S.F. (2012). Thymic stromal lymphopoietin and allergic disease. The Journal of allergy and clinical immunology 130, 845-852.

1. A composition comprising a postbiotic second fermented supernatant of a Lactobacillus species selected from the group consisting of L. casei and L. paracasei, wherein the postbiotic second fermented supernatant is produced by a method comprising:
(i) fermenting a first culture of L. casei or L. paracasei to form a bacterial suspension comprising a first biomass of the L. casei or the L. paracasei and a first fermented supernatant;
(ii) separating the L. casei or the L. paracasei first biomass from the first fermented supernatant;
(iii) fermenting the L. casei or the L. paracasei biomass from step (ii) in a minimum solution consisting of water, saline, phosphate buffer and a lactate salt to form a further fermented biomass comprising the L. casei or the L. paracasei and a second fermented supernatant; and
(iv) separating the L. casei or the L. paracasei from the further fermented biomass from step (iii) to obtain the postbiotic second fermented supernatant;
wherein the L. casei comprises in its DNA genome a DNA sequence 100% identical to one of the sequences selected from the group consisting of SEQ ID NO: 2 and SEQ ID NO: 5; and
wherein the L. paracasei comprises in its DNA genome a DNA sequence 100% identical to one of the sequences selected from the group consisting of SEQ ID NOs: 1-18.

2. The composition according to claim 1, wherein the second fermented supernatant is prepared at a temperature of 0° C.-10° C.

3. The composition according to claim 1 wherein the second fermented supernatant is a dry powder.

4. The composition according to claim 1, wherein the first and the second fermented supernatants, when analyzed by MALDI TOF mass spectrometry, have the following peptide signal profile:

| SN1 | SN2 |
|---|---|
| m/z | m/z |
| NO signal | 357.07 |
| | 423.06 |
| | 373.05 |
| | 406.07 |
| | 439.04 |
| | 489.05 |
| | 505.03 |
| | 538.05 |
| | 557.1. |

5. The composition according to claim 1 wherein the second fermented supernatant, when analyzed by MALDI TOF/TOF mass spectrometry, has the following peptide signal profile:
SN2

| Fractions | m/z |
|---|---|
| 3 | 1193.2 |
| | 855.2 |
| | 756.38 |
| 4 | 524.06 |
| | 542.08 |
| | 664.12 |
| | 756.38 |
| | 644.37 |
| 5 | 756.43 |
| 6 | 610.34 |
| 12 | 1006.8 |
| 13 | 1393.2 |
| | 1153.77 |
| | 1033.68 |
| | 707.28 |
| | 855.54 |
| 15 | 1153.76 |
| | 1393.8 |
| | 1033.65 |
| | 855.34 |
| 16 | 1153.76 |
| | 1393.8 |
| | 1033.65 |
| | 855.34. |

6. The composition according to claim 1, wherein:
(a) the fermentations in steps (i) and (iii) are conducted at a temperature ranging from 4 to 40° C. for 12 to 36 hours;
(b) the first biomass is separated from the first supernatant in step (ii) by centrifugation; and
(c) the L. casei or L. paracasei is separated from the second fermented supernatant in step (iv) by centrifugation.

7. The composition according to claim 6, wherein the Lactobacillus species is L. paracasei.

8. The composition according to claim 7 wherein the L. paracasei is the strain deposited according to the Budapest Treaty with the accession no. CNCM I-5220.

9. The composition according to claim 1 wherein the postbiotic second fermented supernatant is present at 0.1-1% w/w in the composition.

10. The composition according to claim 1, wherein the lactate salt is sodium lactate, calcium lactate, or potassium lactate.

11. The composition according to claim 1, wherein the postbiotic second fermented supernatant comprises 2-6 mg/L of 7-hexadecenoic acid, 2-6 mg/L of tridecanoic acid, 10-15 mg/L of vaccenic acid, and/or 3-5 mg/L oleic acid.

12. The composition according to claim 1, wherein the composition is selected from the group consisting of a cream, an emulsion, a dispersion, a gel, an ointment, a lotion, and a serum.

13. The composition according to claim 1, wherein: the first fermented supernatant comprises 3-9 mg/L of oleic acid, 1-3 mg/L of decanoic acid, 1-4 mg/L of benzopropanoic acid, and/or 10-15 mg/L of citric acid; and the second fermented supernatant comprises 2-6 mg/L of 7 hexadecenoic acid, 2-6 mg/L of tridecanoic acid, 10-15 mg/L vaccenic acid, and/or 3-5 mg/L oleic acid.

14. The composition according to claim 1, wherein the Lactobacillus species is L. casei that comprises in its DNA genome a DNA sequence 100% identical to one of the sequences selected from the group consisting of SEQ ID NOs: 2 and 5.

15. The composition according to claim 1, wherein the Lactobacillus species is L. paracasei that comprises in its DNA genome a DNA sequence 100% identical to one of the sequences selected from the group consisting of SEQ ID NOs: 1-18.


SEQUENCE LISTING (41 sequences)

SEQ ID NO: 1 - DNA, 165 bp, Lacticaseibacillus paracasei
  source 1..165: mol_type = genomic DNA
atgaatcaaaaagctttgaatcaatttcctgaacttacctacacagaacaagtgtcggttgttggcggcgatttgtcagtcgaagtcatcatgaaaggtatcttcaccggtatctttgatgctgggtaccaagtgggtcagtcaatcgcaaaatgggttaagtaa

SEQ ID NO: 2 - DNA, 108 bp, Lacticaseibacillus casei
  source 1..108: mol_type = genomic DNA
atgaaaggtaagcggcagcacttacttttatattttgttctgggtatgatgacaggactggtgacggcagcgatttttcatatcatttatgcctggctttttcattga

SEQ ID NO: 3 - DNA, 462 bp, Lacticaseibacillus paracasei
  source 1..462: mol_type = genomic DNA
atgcccaaaagggtcgatcaacatatacgttcacgccttaaaggctttactttaattgaagtggtggtcagcctgattttacttgcggcggtcatgctgttatggcgaccggttttattgcatgtcacgcggttcacgcttcaagaccatgtgctaatcacgtcattgcaagcagagcatgacttgcaaatgtttgtacgagataaaaagttgcggtctgtggccttaatgtcggtaagggtgagaagtcccgagaaagcttacacgatcaatttttatcagaccaaacattttcgcggtatggttcgtgtgatgggatctgaaaatgggcatatgccattatttacgcatctaaccggtgtcaattttagcaaggtagctcaaggctttcgctatcgtctgtatttgacgacttcgcagaagattgacggtggggtgcaaatcgatgaagatacgcggtag

SEQ ID NO: 4 - DNA, 507 bp, Lacticaseibacillus paracasei
  source 1..507: mol_type = genomic DNA
atggccgctgatttcacccaattacaacaagccattcgcttgctcaatgcccatactcgagctgctgatgagcaagcgtggcaagtgctttttgatcgttggctggcaactttatcctctgaaactcgccggcaaatgcaaacagttcggtttaatcatgcccaattgacgttactcacaacgctggatcaaagcagtcgcaaacaactgcgcaatcaggatttaaccgctgctgttccgttctcacaaggcctagtctcacgctatgttgctcgccttgttcaattaaacttgctgacaaaattatccttgcccgacaatcgcaaggcctacattgttgcactaactccgcttggtcaacaagtcgctgccttacatcagcaaatgcatcatcacacaaatgctcaactcgcctctgtacttcatacccttgatccacaagatgttcaaactaccattcaggtactcacaaaactgacggctcagcctttacatcccaagtcttag

SEQ ID NO: 5 - DNA, 261 bp, Lacticaseibacillus casei
  source 1..261: mol_type = genomic DNA
atgggcggtgtcatttgttacgcggtgccggtcttttggaaaagaatacttcgcagacacctgattcacgagattaagaccctgaatcaaggattgcagttatcaagcaaagccatgagccaattaattgatccggaaaatccttatatggtatttgctgatgaaaatggtgaactggatttttcatttttgtggctaggcaacttgcgtcaattgaggcgtgaactgcgtctaattaaagaacagaaagctagggtttga

SEQ ID NO: 6 - DNA, 1077 bp, Lacticaseibacillus paracasei
  source 1..1077: mol_type = genomic DNA
atgagtaaatataaagttattatttggggattaggaaacgttggtcgttccgcagtgagaatgatcgcggaaagacaaaatatttttgaattggttgcagccgttgacgttgatccaaagaagttaggtaaggatgccggagaagtctttgattttgacaaagtcggcgtcaaagtttcagatgatattgatgcagccttgaaacttccagctgacattgtgctcgacttttgcccaacggaaatggacaaacaaggaacattcatgccttctgctattcgactcgccaaatcgctcgatgccggtaaaaacgttattaccacgattccggtatatcatgttcaagacagtcagccagaagtatatgaatatctaaatgaacatgctaaagcacataatgttgcttttgtaccatttggacttttgccaggcgattatgcctcatatatcccactagttttggccggggccatgggccacgtggataaaattgttgttcaatccggtgaagatgactggcacaacacatcaggctgggtcgatgtcttctcatatggcggcgatatcaataaatatccaaaaccagactcagacgaagatctcttggctaagttcatttatgcttattattcatccggcgtatacgagatggccgataggatcggtctgaaatatgataccttcaaaccagagcatgaagtcttcactgcacccaaagatttggaaacgatcaagggtacagtcaaaaagggcagcatttatgcccacagatttaccatggcactttacaacggcaacgaacaagtagccgccttaagatatgttcataaagttgataataaagagacaccagaattaccgatcaataatacgattcatattgaaggcttgccgtcagtcgatgcgcagatcgatggattgatcccagaaagagaaggctacgtttcatcagccgctccagcagtcaacttgatccctagcattctcgagaccgacaagacaggttatgttgaagtctgcgaccttccagtagtgattgccaggccattggatattggcgcaaaaaaattagtctag

SEQ ID NO: 7 - DNA, 561 bp, Lacticaseibacillus paracasei
  source 1..561: mol_type = genomic DNA
atggcaacctattcgcagatagaactagacataatcaaatcatttaaagggctgatgaaagaccatgaattcactgagatctcaattaaaatgatcgctgaaaaagccgatatcactcgacgcggcttttacaatcacttcttagataaatatgatcttgtcagtaccatctttgagcatgatctttttccaacagtcatcagtttgacgaatatcaatgactgggatcaagggtcgctgtttatcgtgaattatctccaagacaatcgcgactactataaaaaattgttgtcgcttgaaggacaaaactgtttacagacagacttttataaattgactgagatgcagattgggatcttgatcccagaaatattggtcggtaggaaaatttctgacgaagatcaggcatttttaagcgattattattttcacgcttatatgggactgactaccgaatgggtcaaaggtaaatatggtttttcaactcaggagttcgttaaacggtggaaagccttactcaataattcaatgcataattatctggacaactacgctcgatga

SEQ ID NO: 8 - DNA, 1077 bp, Lacticaseibacillus paracasei; identical to SEQ ID NO: 6
  source 1..1077: mol_type = genomic DNA

SEQ ID NO: 9 - DNA, 429 bp, Lacticaseibacillus paracasei
  source 1..429: mol_type = genomic DNA
aaaaacggcttagaacgctcatatttgcgttctaagccgtttttatcagcataggttcttgacaccaataaacatctttagtaattgatcaaatttaggcaatgtgcttttgtcggtgaatggcgatagccctaccgaagcttcagctgaggttcttctgagccacgcaagcgaagcgcgctagggcaagccaacggcgcgcaggcgaagccggagttaaatgtggcgcagccacacctttttagggagcaacgcgaccagaattttgtatggggtttgggaagaggttctccccaaggtcttttgtggttattaacaagcaaaacacaaacacaagcctcgcgcgcgttatatatacttctaaatacttttaaatactttaagtacttagggagacgagaatggctcaaccacgcgtttaaatcgact

SEQ ID NO: 10 - DNA, 241 bp, Lacticaseibacillus paracasei
  source 1..241: mol_type = genomic DNA
acgacctctcgaccacccactgcctcaccaatccccaggtgaaccgggccaagggcactaccgagcaacccgacccctatatcccggtgggcgtggtgaagcagaccgatgggggcatcgtggtgcggggcgcgcggatgctctccacgctgcccacggcggatgagcttttagtcttccccagcactttgctcaaagaagggccgggagccgacaagtacgccgtggccttcgccatccc

SEQ ID NO: 11 - DNA, 7384 bp, Lacticaseibacillus paracasei
  source 1..7384: mol_type = genomic DNA
attgatcgcctccgggtcacttatatgtaactaataatactcccttctctctttagttacaatagggtacagcctatcgaatcacttacgcttccactttgagataacttttcgttattatttatcaacggcattaacgatatcattaactgtttgcaatgcatcgctcagtacactaattggtgcttgttcaatatactgcatgtgtcgttgtacaaaatcaaaggtgtgaaattgtaacggattcacgtacccttctattttttcagtctgaatcggtaccatcaagcctgtttcagctaagcgattatgtttggcatgcgtgatcggacaaaccaacgctaatccagtcattttggcatattgttgattgctaataacgagcgctggtcgtcttttttgaatttcatgaccccgacttggcatgaaatcaatactcacaacatcacctttgcgtggttgataatgcctagtcccactcacttggtaatacctcgttttctaacgtaatatcttgttggtgtacttgttgcttgtaccaatcaccttcaaatggattgcggtgcttcggcagataaagaatgccaccatcatcacgttgctcaactgtaaattcagttccatcggcgattttaatactctttggaatggttaatgtaatggaattgccaacccttcttgctttaactgtcattgaaatcatcctttcgtatacaccgagtatacaccagcgcgaatgaatctgcaactcttgtgccctcttgtgtacaaacaccactgtcaatttacttttgcctattgtgctttatctcttctcgttctgtcattagtatgccaccaacacggccgacttcatccggctcacctttgatgacgccttgaataccatcgatttgcttccaagtctttacttccaagttcttcaaagactttgatatcgtttttcgatgccggcaaacacttcttcattctgatgcctttcaaagaattcctctagatcattcatcagagattctcctcacttaaacctagttggcgtcaaattccatctcagcaatcgaatcttctaggctgtccattacttcgtatgtttcaatgaagttaacccagctgtcatatggatgtagtttgctttctttaatgctagttctaagcttttcgtacacgttgtcaataaactgctcaaccatcctattaaccgcactattaataaccgcttcgttccactgcttagatgctgcacgctttcgattttgactatgctgatgggacagcgcccggcttctgaacaacgctactgccctgctgttagcattcagataggtttcaaattcttcacgatccattacgtttcctcctcaaaataagcctcatttcatagcacagcttcagcaaaaggcatgtcatcctacatgccttttttctgttgctcttcaatatcagtataaaacgtcctgccgctttaggcaaacgtatgttcgctattaagaacatacgtttgtataataactataaaagatttaaaggaggtcaatcgtatggaaaacaatgtcccgcgtgaaaaatggctttaccctgaccgctgcatgaagaaatggctgggctggattctaagcgaccattccgcctatatggaagaagcggctatctcagaacaaccggtgctcccaaagcctgaacagacacaagaaaccattaatggcgtactcgaagatgcttggcaaaactcaaaaattgtcgcagttcaaatcggtacgccatacgatgatcttctgttaccggatattgaaggcgccgtgattggtcattgggacgctcaggtttatctacagcttaaaactggtgagatgcaatccattaatgcagcggacattcgcaatgtgcaactgctcaatccagatcggtggtgggcgttagtatgacgacaccattagatgatccaacaaggttaccggtacacgacattatgtgcattgactgtaagtccttttacgcctcagttgaagctatcagacgcgggattcatccgttagccgccgacattgctgttctcagcaaaggtaattctcctggcggtttggtgctggctgctagtcccaactgcaaaaagcgttaccacgtaggactgagtacacgccgttttcagctaagggacgatatgtaggtagaacttgctgaaccgcggatggctaattacattcgcaagaattacggtatcaatcgtatttaccgtcagtttactgacgatgctcactggtctccctattccgttgacgagtcctttattgacgttacccacccccacaatctcttcggttctaatgaagaaattgctacccaaatacagaagaaggtgtttgatcagtttggcattgtcacaacagttggcattgggcaaaatcccctattggcaaaattagccctagataacgaggctaagaagtcaacgccttggcaagccacttggacttacgatcgtgtgccagaaacaatatggaaacttgatgacttggttgatttttggtcgattggtaatcgaactgccaagaagcttaacgcgattggccttcataatctttacgacttggctcatgcagaccgcgccattctgcaccaaagattcggtgttctcggtgatgccatgtactttcacgcatggggtattgattactcagacttaacccgccgctacttaccacgggccgaaaataaaggctacggcaatagtcaggtactcatgcgtgattacactcaggcaagggagattgaagtcatgcttagcgagattgctgatcaagtggctggccgaattcgccatcaccaagtccaaggtgaggtcattagcgttggcattggttatgctgatgcagaagaagctggcacctccggtttcggtgcgcaaatgaagattgatcccacaaatcgcacagacgatttaattcgcgctactcgatttctcttccatagtaagtggaacggacacgctgttagaaatgtctcagttcgcgttaatcgcatcagccaagcaagtacaatgcaacttagtctatttgaatcagcagagaaggaggaagcaaacgcggctcctatgctgtaattacggataaaagaatcaccatcattaggtttttcgtctaacaattttaggaaacttcactttctaggtcgtaactttatttttgcaatctagggttttttaaatatatacattttagctcgtttgtgtttaatattataatcacaactataccaatgataaatgtctaacataaatatacaaacatgttgacagaagctcttgaatacgtttacaattatttcgttcaggcgagctttgtttttgaaaaagtattaatacaagataactaggttagtggctgttgaattaggcccccgatttcgggaccacgacagtcacttgatactcgatttttatcgtttgctggcttgatcgtacattgaacgaaattggtacagaaaaaagagctaagagccgctccaaattagccaaaacgattgcggcgtcaatgcttacggcgatcgtttctgcagttttagctgttaccagatcaagtcctagtttccctttgatgaaggcaaactcacgctcgatctcacctcgtcgattttcggcttgtcggtctgccttacgtttggccggatcgacctgcttcggccgacggcccaatctaggaccgctaagtttgatcccaagatctgcgcacagcccgatattcgcccgagtccgataaagcgtatcagccaagatctcatccgggtatgtaccatacgtgtcaaaataatggtcgatcgttgctggtaagtcagcactttcgttaaacgcattgaacgcaaaccgttcaacggccacgacgccatgactgatcgatacgtcgatcttgggcccgaattcgaccggatcctttgctttgccgcgaatgatcggtcggatcgctggttgatcaaggcttacgatccgatccgcgactcggtgagtgtgctgtcgatacatttcagtttgttgctcatacaattttcgaatgatcgttaatcgttgtgtctgccgttgattcaattgcccgccttgtgcttgcagttctttgacgtaacgcaagtcacgtcggatgtactgtaattgagccttgatctgcttatgggtcgttttcacccaacggcggggtttacgtgaaaaggcggtccacgtttggtgggccttgcgcttataggtacgcggcggtttgaccgctaattgcttggccatggctgcgatgaatcgctctaaattgagccgcgcctgattgagtagctgcgtatcctgcggatacttgatctttactgggaccgcagtcgcatcagtgatcaagatcttctgatggccaagtttagcttggaggcgatcgcggacaaaatcgctaatgatgttcgtgatcaactcggaaagcggcgcgatccggcgcctgaaataggacagcaccgaaaatgaaaacggtgcttgcggctgatactctggcaggccaataaaatactgataagccggtgtatcgcggatcgctgcgactaactcacggtccgatagctgagtgcgctgcttgatcagttgggcgccataaagcagccgaaagggtttacctgcccatcctaagttagacgggaaagccaattggtacgcctcttctagttgcggccacggaacttggtcggccagttggacccactcgttatctggacttaatggggtgcttaagccgctaccaaacgatttgatcgataattgaacggcttttcgacgataaaccatgatccatgcctccgatagggtcgtgtcaaatgcaaacgaaatgagcacgatccgtaaattcatatgcattcattatacgacgataacgggttcaactcgcatcaaatgtggttatatcaaattattcaacagccactaggttaagatcttcatttaagtgatattcatttgcaagcaattgaaaattactcatcacgaagaggatttcattggccatattggatagcacgcaaatcacttgctttaagaaaatcagttcctttaatgagtctcttaaaggacggggctctcacttgtactcacaatcaatgttaactggagatcaacaatatggtcatagattcccataataacattgacttgactatctaaaagaggcttctaactttgatattggtggggttattggttgcttggctgtaagcagataatcttaacttgggttattttcattgtgttgtaaagacatttgttataaaggccgaagttatcgctttgacttgtaataaattatttttgattgagatatcagaaaataaacgggggataataatgaaaaagattattaggattgttctttgtgttgttagttgcgttagtatcatggtcggatcgcttgggttctattcaactccaaagatcgttaaagccgacagtacatctgttacggatgtcgacattaatacctatatttctagcatgacacttgatcaaaaaattggacaaatgtttgtagcacgaacctcacaagatactgataaagctcgtgctgatatagcaaaatataatcttggcgggctgattgtttatggtgttgatttcactagtgttaaagggacaacagctacagaagctcagaataacttcaagatgaagatgcaaggctttcaaaactcggcaagtctgccactattgattggtgttgatcaagaaggaggggcagtctcacgcttatcacaaaatcctctaattgccaacggcagaagttttccttcaccacaaatggcttatgctaatggtggaatgaccaatgtaacaaaagaagctagtgaagtcggaactattctaaaaaatctgggcattaactggaactatgcaccagttgccgacagtacgcctgacacctctagttttatttatggtagaacctttggtcaagattacttggctactgcaaactatattacgaatgtgatccctgcgtggcaaaatgctggcattgccgcaactctcaagcatttccctggttatggatccgcgattgatacgcatacggattttgcagtcgttacaaagtctaaggaggattttgaaaaagaggacttgcttccctttaagtccggtattacagcaggggcagattctgtaatgattgcacatatagtaatgcaagctgttgacccagtgtatccagcatcattatcacggaaggtcgttaccgatttgttgcgtaatgaacttgggtataatggcttaataattaccgatgcattggaaatgggggccatcaagcaatttgctcaagaacatgatcaagttcctgttgatgttcttgctgttgaagcagggaatgattgcatcatgaataacgattatgaaaccgctattccacagattcatgcagcagtaactaatggaactattaaggaatcagaaatcaatgaacacgttttccgtattcttgatctcaaacgcaaattagggttgttaactaaaggacaacttcagcaaaaaaaagttcaggttgacaatgtttcctacagcagtgacaacaaaaaggcaactgtgagtggaacagttgttgatagtgattggcaagttggagaaccattatcggttaaagactcgactgggaaggtcattattaccgcagacgttggtgccggtggtaagtttactttcgatgttcctactaagtcccaagaacaagtattaactctgactactaatttacccaacatcgctgattctcaaataactattaaggctgtgagttcatcgaatactaacaaagctttgctagaaaacttgatcaacgctgctgaacagttggatagtaatcaatatactgtcaagtcgtgggaagaattacaaactaaactaactgaatcaaaatcgattctgaacaatgatagtgctacacaagatcaagtagacgcttccgttaatgctctacaaattgcccttaagcaattagttcctgtatcaaatagcggaaataatggtcaaagctctaatgatagcagtaaccaaagttcatctagcagtagtggcaaagaatcatccagcaatagcaatgccaatattactagtaaggatcagtcagctaaggattcaaatacgaggcctaaagaccatagtcttttgccaagtacaggtgaacgggtgatgacgggaatttctgttctaggggtaattttaatagcttgtgtgactatattatatattcggaaaaaaggacgcagcttttaattagtctctgcgtcaactggcgttaaaaactagattgaagtaataaagttaccacctggaaagaggcatgctcattgcttgcaagggtgtcgacgtgtaatagaaaagttgggg

SEQ ID NO: 12 - DNA, 1410 bp, Lacticaseibacillus paracasei
  source 1..1410: mol_type = genomic DNA
tggcgtgggcaacgtgcacgttttctagtcgctaaactgtgaacaatgctcgtgctaaatgcaaaactgagcaaggagatgaactataagcgggggaccctttgctattgaggaggaaggcgaagtagagaaagagcggtgatttgaactcgaaacagcggcgccgcaggctagcagcactgttagattaatcgccagcagcagatactagacagcttcttaaaggcttgataatagcgttgcgccatttcaatggaactagtggtcaaaatcgcattgtagttgccatgacccaaactcgttttacgcgggcctttttgtaaaatatattgaacaacttggttaatatgttcatcggtctcaaagtcagccggagttaaatacgtttcttccaagtccttcactgacatcgcttgaatcttggcttgaattttcgcttcatcagccgtactcagcaggcggccttgcttatcacgacgagctttaacgcgctgagtttctttttcaagggcccgggttattaaagcgtctttaccaatcgttgtcacgtgttccacattaaatggtaacactgcttggtcctctaaggcgtctcgcaagttataaacatgacagactttaccaaatagctcctccgtcgtaactgctagatcacctttgagctgtttcttattttcattaaaaatgggggtgccagtgtaaccataccagttactattgataaacgctgctcgaatttccttttgcatcttaccaaactgcgaccggtggacttcttcaacaaagaagatcacccgttgctttaaagtcttactaaagcgggattgcttaccggttgccagctggacttgcgtttttttgaccgcccgatggagcttttgaatcgaggtgaccaagaccttaccgtcattttgttgcaatttacgcattaaatcaccggtgttttgggcttcgttaatggcaatatcatcattggcagcataggcactaaagttgctggttgtctgttcgtctaaatcccgccggtcaactaagaagatgaccttatcgacaccaggatcttgcgcagctaatttagcggttttatatgaggtgagtgttttaccagaacccgtggtatgccaaacgaaaccatcctgatggtcatgaatccggtgcatcacggcttcaatcgcataaatctggtaaggccgtaagagaattaagctttgccgctcttggtcgatgactgtatattcactgaccattttgtgggccatgggaatattaaggacttggcgcgtgaacgctaacccgttttccacggggtgattatcccgcgtccgccaattgaacaaaaaggctttattgaaatgatccggttcggcattcgcaaaatacgccgtactatccggcgtcataatcacaaac

SEQ ID NO: 13 - DNA, 592 bp, Lacticaseibacillus paracasei
  source 1..592: mol_type = genomic DNA
cgatctaaaagctaagttattttccaagatatcaaacaacttcttaacccaagaatcttccacacataggacaataatgaatccaaatagattcagctttttcctgcaaaccgggatcagtataaacgtccagtaccggataatcacgcattaagttcagctgccaatgggtatcatctaaattaaaaagatccgatttagtgtctccccttactacattatggcaatacacacaactgttgttatacatgcttccttgctttttgattttaaactcctccattttgcatattataagaagattacttctacttgatatatagatgctttccttgcgagggtaagtcagacaaggaagcatttctaacttgagatacttaagcttgtctcaatagatgtagatagcggctccccaatcggatattaacagctcaactagtcaaaccagatatataaatgtgacacaagctggaatatatatcattatctagataattcaaattgagctaataaaatcaataaagaaaattttaaataacattattttataaacccctttaggattttcccgatttgatattctacgtatgtt

SEQ ID NO: 14 - DNA, 2233 bp, Lacticaseibacillus paracasei
  source 1..2233: mol_type = genomic DNA
gagtatccaaaaatacgacgggtatttgaataggatacttattaagcgagaatggtattggaaatctgtggcagccactcagcggaaccatacctttatcccaaccccacgcaaaaaaaacatcaagtaatccgtcagatatgatgacttaattgtgggacagttctaatatgaagaaaacaggttagataattggggtgaaaagatggcaacctattcgcagatagaactagacataatcaaatcatttaaagggctgatgaaagaccatgaattcactgagatctcaattaaaatgatcgctgaaaaagccgatatcactcgacgcggcttttacaatcacttcttagataaatatgatcttgtcagtaccatctttgagcatgatctttttccaacagtcatcagtttgacgaatatcaatgactgggatcaagggtcgctgtttatcgtgaattatctccaagacaatcgcgactactataaaaaattgttgtcgcttgaaggacaaaactgtttacagacagacttttataaattgactgagatgcagattgggatcttgatcccagaaatattggtcggtaggaaaatttctgacgaagatcaggcatttttaagcgattattattttcacgcttatatgggactgactaccgaatgggtcaaaggtaaatatggtttttcaactcaggagttcgttaaacggtggaaagccttactcaataattcaatgcataattatctggacaactacgctcgatgaattacacagattggattaaatgagaaagatgttacatttgtgccaatatgtgaattgataaatatttcacaaggaactattctttccctgtaaacgaaagttgacttgaaaggagttagttctgatgagtaaatataaagttattatttggggattaggaaacgttggtcgttccgcagtgagaatgatcgcggaaagacaaaatatttttgaattggttgcagccgttgacgttgatccaaagaagttaggtaaggatgccggagaagtctttgattttgacaaagtcggcgtcaaagtttcagatgatattgatgcagccttgaaacttccagctgacattgtgctcgacttttgcccaacggaaatggacaaacaaggaacattcatgccttctgctattcgactcgccaaatcgctcgatgccggtaaaaacgttattaccacgattccggtatatcatgttcaagacagtcagccagaagtatatgaatatctaaatgaacatgctaaagcacataatgttgcttttgtaccatttggacttttgccaggcgattatgcctcatatatcccactagttttggccggggccatgggccacgtggataaaattgttgttcaatccggtgaagatgactggcacaacacatcaggctgggtcgatgtcttctcatatggcggcgatatcaataaatatccaaaaccagactcagacgaagatctcttggctaagttcatttatgcttattattcatccggcgtatacgagatggccgataggatcggtctgaaatatgataccttcaaaccagagcatgaagtcttcactgcacccaaagatttggaaacgatcaagggtacagtcaaaaagggcagcatttatgcccacagatttaccatggcactttacaacggcaacgaacaagtagccgccttaagatatgttcataaagttgataataaagagacaccagaattaccgatcaataatacgattcatattgaaggcttgccgtcagtcgatgcgcagatcgatggattgatcccagaaagagaaggctacgtttcatcagccgctccagcagtcaacttgatccctagcattctcgagaccgacaagacaggttatgttgaagtctgcgaccttccagtagtgattgccaggccattggatattggcgcaaaaaaattagtctagactaggctttcgaagctgctttgaccattaaggttggagtagctttttcatttgcaagtaaatcattacggcttgtgtatacggtatacaaaatggagaaaacgctgactagtttataaatcattgagacttaacggccggataaatgctgatctgattatagaaataacaacaaaaaggccacgctaaaaatcatattaattataatcgggaaatttattaataatattcaagaaaaataaaaaccgtgggtacattatttaaaa

SEQ ID NO: 15 - DNA, 5866 bp, Lacticaseibacillus paracasei
  source 1..5866: mol_type = genomic DNA
tttgaaactaagacgaaagctgccatgtcaaacaaagccgccataaatgccactgtcacagatccatcagccgcaatgccagcatcttgctgaagttctttaacagcattaagggtgttattcgtgaactcattattaaagtctactggactaatccctttgcaccagaaagccccttgaatgagttgagcaatgttccccttatatcctggcttcagactacctacaacaggtgctaaggcgtttttggtcgtctcgccaaagccttcaccaatagcactaataccgatttcgtgctgtaatcccattcttaggctataaattgttggccatcccgtttgcccgttttctggagctgcgacaaagccaggaacgctaccatacgttttgttgagccatttttgaacggctcgtactgcttcatctgccattttaaagtctccttttttgttttagacagcacgtctgccgtcacaaaaagcaaacatatgttcggattcatttcatctcttcaaagcttcgaaaggcaaccctgatccacaaataatccttttattttgaacttagcaaaaaaatgaggccctcacatagtgttgaagttgcctcattcttaatgtctatatttaaagtattgccacaacgatggatcatcgaacgctcatggacttggttagacatgtatcgacgactatgaaaatgtgggcgcaagctcaatttcaccttccaaatgtttgtgctatctcatttagcgctggtttttttaggatagactagacaaggactaataatttctcaagaatcccgcaactccactattcattcgtcgaaatccccactgatactcttgtccttgcacgttcgaccaagcaagaatgtttattccgataaccgaattgttgccatcaagtaatggacctcccgacatgccatgatacgaattaatttgttgtgaaatatatattcctagtgggtcctctgaaaagggtgtgacagtcccacttgattgaaccatgactccttgaagttcgtaccctgattgcggatcgccagggaatccaatggatcttgctgccatcgtatcagcagggttcgtatttaaattaagacccgcaggcatactaccagacttcatagagacaattgcagccccgtaatcattcgaagtagctgttgaattattaatccatgcctgtggcactatcaatctattcaatactccgtaaccgaccccttgatgatttgcttgactatcaccaaagttaataattcctccagaaatataatgaccatcatataacatgtgtgctgctgtccctatacggtctactccaatgctaaatccagtaccgccagaagttccactgctcagctctgtgccatttgagttagagatgatttttttatacgaactgtcaaggtatggtgagttaatgaccatgacaccatttgccatcgaaaaccacgtgctcaaaaccccaacggaactgtacggcgccgaattggggtttgacacaggtgataccgtcctcacagatagatgattagacgatttattcagtttcgcaagatattcggacgtaattccttgaactttctttccttcttttaaatcctgatattgatcaggtgtataagctttgacactccctttaaaatcgggataagaatactgatattgtcgaatgatttcattcaaaaactgttgtgttgtcgtctggttagtcaaaacaatatcattctttggtaaaacatggctattcgctgccaaaggatttgccatactatcagcacttacacttaccgtttgaacttgaattatcgctaaggctgcagctatcatagtgatatatgcccatacttttcgcaatttaattccccctttttcttaaaatgaaaccgcattcacggaggcttgtcaatgcttttaaaaaacaaacgttacttttggctcatcttggctgtcagcataattggagtaattgttcttgcggtgttatggcgcatgaaccctgaaggaacggcctcaaataagtttgaacgtcccaccattactattaaaaaagtcaaacttattaagcacagtaacagtattgctgtcacatttgctacctctccaaaaagcaaatatacgataagtgatcttaaagagaatcaactttcttctggcatttcgaataaaagagaaaataccgtttcggaattaaagccctcctcctctaagcttgcaatacgggttaagcataacaataatatacaaaccaaagtggtttctgttcccattggttatcatattatgaaaagtgccatttcaagaaagccaattcctatgggagaagagtttaagtacaatggaaagtcgcatgttttatttagcatgaccattacccctaaaaaacaaaacaagaacagtataaaaaacaccactgcttttaacataacggttaaaaatgatcactaccttgtccctgtcgtattagataccaaataccttactgtttctgattcagaaggtaactcattaaaagtaaagccattctcaaaaatttctattccagcaaaaaagaaaaagaccattgcaataactattgagggcgttcccgcaagctctgccaatggtctagttataacgtataatactgtcgatttagacttaccaatctcctttataaattcctgaaattacactaactgtcccccaccttgacagtcagtacactcaaactgtctcttatgcttacaaacacgtaatttaggcggtttttaagcaaaagtcgttagttttcataaatgttatcttatactctaatgagatctagcttgtgataataaggctgtttttctttgacagccttattaagcacactaatcaatgtcaattcgaagtttttggtttcctacttggccaactttgttatcagaaattccaaaactcattgcctcccgccaccatatatttatcgagccattttgaaaatgaaaaatcgaaatatcggtctgcttctattccgggatgagttagatatgattttcctaaccgatacttctctatatcaatatacatatctccgacatcccttatatggagaatgggtactttatttacaaaacttttaggcagtgcctctttttctttaatcatttttctaattgaatagacttcacacgtatatcccataggaatcccttcgattgttgtgtcaaataaaaatagtccattagtaatcgagagaaactcaatgtaatcctgtggaaggttccacctttttattttttctatatcatcagcgtgtgcaggaggttctatcttaaaagaaacattttgcacatctccatctaattggaatgttgagagcgctttttccccatttttcgtcacctttattaaagaattaattcttcgccgaattagagattccaaatgagttcctcctcaatagttgttaaaccacgcggtgatcaaccgatgatttggtgttaacaccggcatcaaattattaaagtcatttgttccgccataaactctcgggcgaatatgatgcacttctcgagaactccaaaaatctgcagattgattgccatatgtttcactgaacgttttaatataaatatatctatcttttgatgaccaactaggagattttgataacttagtccaggtagtattaataggtgtttctgcatcttttttagacacgggatcaacatattcagggaaattctgtcctattttattttgtaaatagatggccgttggtgggattggttttacattagccgctccattaactcctataaatccaacagcgccagcaacgttaaaaaatgtggttttggctggcaattctgtaaaaccaagtaatcctgtttgcaaaccgcctattttgctaatgggtgccagcgtattggcattcaccggacctgcaagggtaggctcagtattaaattcaatttgcacatcaactgcggctggcgggacaccctcaatagaatcaatccagaaattgactctgaattttccggatatgtattcttccgataagtgccaggtaatatttgtaacaggaacttctgcacgagaacttatgccattgccatgactattattcagtaccacttgttttccagaagcgataattggttgtccattttctggattacttcttgataacttgtcagcattggtcagttgagtattgttatcgctaatacggctttcagttgaaacaatttgatttaaagattgtgttgtgtctgcggatacaattgttgaatacccaagcaaattgactagaaagagcccgaatattagcccaataactttccactttttcacgcctattatcttctttccaaagttcttcagtgcctggcaataactgtatacattgagcagtatagtcgctattttatagctgaacaactcataaagctcaattattattagcctataaaaccactgcctaagtgaattgatctagaacgaagcacgccgaagaagtcgctaaatgtgctaagaaaaatgtgcttgaatagctcaaaagtaattagcgtctccattgaaaatccgttatttttaagtgatctagtgttaactatgaatcccaaataaaaagcaaaatccgtaaatgccaaattttcctttttgacgtttttctactgtcgcgagatttgcaagtgtacgtacacttacgatgaattgacagaatctcagctgcgctgatcgtcaattttgtttgggggcacgcccccaatccccctgttattttgaagggaggtgagtcccccttcaaaatcaaaatttaaacagcatctgccgccatcttttcgctgaccttctcacgatgttacacgtggtgttgacacccacttgcatttagagtttcattcaagttgaacattgtgtaatatatgagttgcatttgataaacatatcagttgctatttgtgcaactttaaagcttcggctaattcaacgttctgttaatttacaagcatctcgacagtttctgttaaagcaacatctacgcttcaattcgagcaactcactatacgtatgccgagttgcagacaagctactatatagctgtacgcgctgaaacaccaaaaatcgttcgtttatgcccaataagcgaataatcttgctcaggtgtagtaaaaaactgtttacgtgtagtgaatggcgctagcccttgtcgtaactggcatcatccacgtgtagtaaaacgcgttttactacacgttcgtaattttttcacgtggagtaaatggcgttttactacaccttttgaccccaacgtgctatcacgacaaaccaaaccgcactgcggtttaccccaattttggggtcagttttgccttatgctctttcatgattttaggcgcgttccaagcagtctcaaaaagtggtcgatccaggcgagccgatttttgagaaggattggatagcaactcaatttattttgatcttttgcttggagaaaaacgttcacgttttgaccagggccgtcgcaactgttgaccaaaactcgtccggtaacgtgacgctatttaaacgccgcgttggtttgctagacgaccattcatcatcaccattcaggaggtttttgaaatgacaaagcaagacgaaacacaccgggtcatgttcactttgaccgatcaggcgattgcaaaattgaatcagctggtcgcaaaaaagcaacaggaagtgaatcaaaatccggaactggctaagtaccatgtcagcgtgaccaaatcaaatatcattgaggactggttatcaaagcagtgagtttaaaaagcgctaaagggcctgtactagcgtttcttactctggtgggtataattaatgctctctacatcaaaaacg

SEQ ID NO: 16 - DNA, 11007 bp, Lacticaseibacillus paracasei
  source 1..11007: mol_type = genomic DNA
gccacgaacctgtagccgtttggatgaagccatataatactggaccaaccgccgcaaataagtagccgacactttgagcaaaatcaggaatactaatctactttgcccttaaaaaatcttgagatgatccatatcttgttttgccttcattactgtagttggtcataagaagtgccctacattcattagattacttgtctaataattgtagggcacttgggttgagaaaaatgatgttaactaagaatgcaaacgaactaaaatctttgcttgctttttatccttttctaaggattcaattccttctgaaactaattcatttaattcaatcttttttgtaatgacctgtttgaatagtgaacgatgggtatctataatcttaattactcgatcgaagatattggcatatccataagatgttaataaactaccaccttttttaagaagagctctaacatctacaactggtggatgttgaaataatgcaatcacggtaaccttgccaccatttttaagagcctgaatggcaccagtaagtgtgggttgtacaccggcgcaatcaaacgcaatatccactccctgattttccgtgatagtgctgatagcgtgagctaatgacttttgactatcagcacgtattgggtattgaattcctaattcatttgctaaattcaaacgttcctctgacatgtcatttattatgacgtgatgtgcaccagaaatttgtgctattaaggccgtgaacaatccaattggaccagcaccttgaattaaaacatcatctccaggagacactcggcttgccataactgcctgtgcagcaactgaaactggttcaactaaggcccctaaatcaagcggaaagctagctggtaagagatgtgcaaaggtactttttacattgcacttttcagctaagccaccgttagccgaaaatcctaagaatcctgctgattgatcactacctatagcatgttcacaccaattataatgaccagaaagacattccggacattttccacaagcaatcattggttcgactgcaactttatctccaattcttaatttagatacttgttttccaattttagaaatcgtcccagaaaattcatgaccaggaattagcggggcttgcatatgggttagcggatgaggtattgtcgccaaatccataccctctaaatattcatgaatgtcactaccgcaaataccattaaatgcaacctcaatttgaacttcatctggtgcgggatcaggaatatttcttttttcaaagcggatatccttaggaccgtaaataacagctgccttcaccatagtcatagtgcttcgcttccttcatgttcaatatagcacaatcgtatataaaatagtgaatagatttcagtaatgaagttaccatcttgacttaacaaaaacttgctaactgattatatgagaaacttttacttgaaacatttttggtgattaccattaattccctcggacatattttgaaaaaccctatttgatgctgattgcaaattattttatgcgtatttattaagggtttctatgttgaagtatatagcaaacttgttcaagtaactgactttcacgtgggctttagccaagagatgctgagcagcgaacccaaggggtgttactcgcccacgcaaaaaagaaatccaattgcattccagtatgagcgagaagcaagccattaagacgctgattcatgaactcgcgcacagtgaattacattgtgatccgaagttaaaattggatcgttcaaccatggaattggaagctgaaagtaccgcgtttatcgtttgtcaacatttgggaattgacacgagtgattatacgtttccttaccttgctgtttggtcgaaagataaggatctttcccagctctccaaaagcttaacgcgtatccaatccaccgtcgaaaaattcaataaaaccgtcgatcaaaaccttgaaaagattcgtgagaaaccgttgacgcttgatcaaaaaatagaacgcgctaaaaccattgcgacaacggaaaacatcgcaaaaaaagagcaagggctggtgcaagcaacgcaggagaaaacacgctaacccatttgttgaatactctcactcaagaggacactccagcccttgatcacccaagaaaggaattaccaacatgaaaaccattgacgaaatgaacgaattcgatcgtgacattatcttacttcaccgcaagtctgtgagcgaagatacaccgcaggcaattcttgtgaaagtgaaacagattcgtaacgcaattgccgacgaaaaggcgggtaaagaagatccaattgagaaagaatttacactcgaatgttacgacgaagcaatcagaaaactaagggacctttcggtcgctgattatcagttgtggttgcgtcaaaacaaagacctggaagggtttgaattttgattttgaagggtgtcgtagaccccttcaaaataacgggggattgggggcgtgcccccaaaacaaaattgactatcagcggagctgatattggatcaatttatcgcaagtggacgtccacttgcaaatctcgcgacagtagaaaaagccccagaaaagcaaatctgaaaaaatgtaacaggcacttgatatcaagtgccttattgtttctaggatcgctaaaaataacaggaggtggttacatgaagcaatctgatgaacaccgcacgcgttcagtgagaagcactgtgcgtatgaccccagaggagcgtgcttgggttgatatgagaagagcctctgtcggcaatccaaagttcaatgcatttgcctgtcgcgcactcacgacgagcaagatcgttcatgtacattttactgatactaaaaagttacttagacagctgtcaagaattgggaataaggctcctatgctgtaattacggacaaaaatagtttgtgcgataattacagcataagggcctctaggtcggagcccaggaggcggagaccgccgcacagcccaaccccacgccgaaccggaggccagcccgcccgcaccgcggccgcaatcatccacccaacgccccccaagtttttgatagcggtaacaacgcctgtgcgcttgtcgtggccggccttttttcataaggttggaggagaaaggaagggtggttatgggcgcttggtatgaacacgcaattatttaccaaatctatccaaaatcgtttcaagacagcaacggcgacggcatcggggacctgaacgggatccggcaacggatcccgtacctgcaagccctcggcatcaacacggtgtggctgaacccgatcttcgtctccccacaggtggataacggctacgatgttgccaattacttcgccgtggacgaaaccatgggtacgatggccgacctggaggcgctgatcgcggctctgcacgcggccggcatccgtctgatcatggactttgtgctaaaccacacctcggatcagcacccgtggttccaggacgccattcacgccaaaaatagtctgtaccgcgactactacattttctctggccacgacgggcagctgccaaacaactggggcagcttcttcggcggatcggtttgggcgccggatccggcgggaaccgggcagtcgtattttcatctgttcgaccggcggatgccggatctgaactgggccaatcccgaggtgcggcgggcgatgggagacgtcgccacgttctggctcggcaagggcatcgacggactgcggctggatgccttcatccacattgccaaggccgatctggggcaggattaccccctggctccggggcagcagacgccggtggtggcggagccgtttttctccaacctcccgaaggtgcaggaatggctgcggccgttctgcgaccggatcaaaaccgactaccccgacgcgtttctgctcggcgaggcggcatcggccaacgttaacctggcggcggattacaccgcgcctagccagcacctgatggacagcgtgatcacgttccgctacttcaccgaggacgaaagcggcctggatccgcggctgccggcgcagtaccagccgcggacgctggatttcccggcgttcaagcaaacccaggcggtgtggcagcagaccctcgccggggtgtcgatgccgacgctgtactggggcaaccacgacatggcccggctggcgacgcgggtggccaaaaccaccacccaggcgcgcagtctggccatgctgatgtacctgcagcgcggcctgccggtgatctactatggcgaggagctcgggctacacaacctgcagttcgatcacgttgatcagtttgcggacgtttcggtggcgccgttcgtggccgcggtcgaggccaccgggcagtcgcggagcgcggcgctggccatggtgtcggcgacgcacaaactgccggcacgggggccgatgccttggacgaccgggttgcaccagggcttttccaatcacctgccgtggctggttgggcgcagcgaggacgtgaccagcgtggccgcgcagcaggccgatgaggccagcatgctgcacttctaccaagcgctgattgccctgaagaagcagccgctgtttcaggccgggcattaccggctgctgacgacggcgccgaacctgtacgtctacgaacgcacgctggccagccggcgggccctggtggcggtggccttggatgagcaaggcgccaccttcaccgtgcctgaaggcctgacgaccgtggcgctggccgccggcgattaccaactcgaaggtcaaacgctcacgcttggcgcgaacgccggcgtggtgttaaacgaaaggggaactcgataaccatgcaacttgcagcattacggcaccgcccagaaagcgaagattgttttttgtacactccagatgagctgcggctgcggctccacacagccaaggccgacgtgcaggcggtcatcgtactgtacggggatccgtatgtcaccgcgccgaacccgaccaccggagaaccggaattcgcctaccaagaggcggcgatgatcaaaaccggcaccggccaaaccagcgactactggaccatcagcctgaccgcgccttatcaccgcctgcagtaccagttcctggtgaccggtcaggacggcaacaccgtcctgctcggcgaccgcggcttgcgggccgacagcgccgccaaccgccgggccgatctgttccgggtgccgtacttccacgccatcgacacggtacagacgccggcctgggtcaaggaaaccgtgtggtaccagatattcccggaacgcttcgccaacggggacaagacgaacgaccccaagggcaccaagccttggcgtccggcggatcacccgggccgtgaggattactacggtggcgacttgcaaggggtgctggaccacctggacgacctgcaggcgctcggcgtgaacgggctgtacttctgcccggtgttcacggcgatgtcgaatcacaagtacgacaccatcgactacttcaacatcgaccctgcgtttggcgacaaggccttgttcgccgatctggtcaaccaagcgcaccgccgcggcatgcgggtgatgctggacgctgtgttcaaccacatgggcagccgcagcatgcagtggcaagacgtgctgaagttcggtccgcagtcgcgcttcgcctcctggttccacatcaaccgttttccggcggcgcccttcgccgcgccggaacagggcggcgtgccgcagtacgacaccttcgccttcgaaccgcacatgccgaagctcgacaccagcaacccggcggtgcaggactacctgcttgaggtggcgacgtactggatcaaacagttcgacatcgacgcctggcggctggatgtggccaacgaggtggaccatcacttctggaaacggttcaatcaggcaaccaaagcgctcaagcccgatttcttcgtgctgggcgaggtctggcactccagccagccgtggcttaacggggatgagttcgatggggtcatgaactacgcgttcaccgagcagatcgaggcccacttcctgaccggcaagctgagtgctcctgagctgacggcggcgctgacggatcagctgatgctgtaccgcgaccaaaccgaccaggcgatgctgaacatgctggactcgcatgacaccgcgcggctgctaacggtggccggcggcgacgaggacctggccctgcaggcgctggccttcaccttcctgcaaaccgggatgccgtgcctgtactatggcacggaaatgggcatggccggagaaaacgatcccgactgccggcggccaatggactgggcccagctgaagggcccgatttggcagcgtgtgcaggcccttgtgaccttccgccgcgcccagtcggcaacgctaggcaccggcaccacggcgctgagcgtgaccgcagccgggctgcttaaggtaacccgcacaggtgagcacaccgtgacggcgtattttaacaccaccaagcagatggcgacactgaccgtcagtccattactggcgcagggttacgccggccagcggctggcgccaaccgggtttgctgttatggttcagtaagattatgttagcggtaacaggcaatttgaccttttaaaagcgttttcatattatcataatcaaaagtgtagaaaagttcaggtggcgcaattcacctcccgaaagtgaaggatgcaagatgaaacggatatttgaaatcgacccgtggctggtgcaaagccaccaattgaaccccaacgagaaacgcctgcaggaaagcatgaccgccatcggcaacggctacatgggtctgcgcggtaacttcgaagaaggttacagcggtgatcacctgcaaggcacgtacctcggcggcgtctggttcccagataaaaccgtcgtcggttggtggaaaaacggctacccggattacttcggcaaggcgatcaacgcgccgagcttcatcggcatggcgctcaccgtgaacggcgagcgcgtcgatctggccaccagcgtctaccgcgatttcaccctcacgcttgacctgcaccagggcctgctgacccggagcttcgtgttcgagggcaaaaaggccacggtgcgcttcaccttcaagcgtttcctcagcaacgtaatcaaggaggcggcgctggtgcagctcaccgccgaaagccttgtcggaccggccgagctgacggtggccgcacagctcgacggcaacgtcacgaacgaggacagcaactacgacgagcgcttctgggcaccgcagggggaaaacgccgcggcaggcaccatccagctgcagaccaagcccaacccgttcggggtcccgcagttcacggtgctgctcaagcaaagcctgcgccaaggggcaacccttttacccggcaccgtgaccaccagcaccggccagctgaccagcacggtcacgctgccgctggcgccaaacgtgccggtccagctggaaaaggacgtcatcgtggtcacgagccgcgacgtcgcccctgaggcccaggccgaagcggccgcggagctgatgacacagctgcagggccaaagctttgcggcccagctggcggcacacaccgccctgtgggccaagcgctgggcccaaagcgacgtggtgattgaaggcgacgacgcggcccagcaggggatccgcttcaacctcgcccagctgttcatgacctattacggcgacgataagcggctcaacgtggggccgaagggtttcaccggcgagaagtacggcggggcgacctactgggacaccgaggcgtacgtggtgccgatgtacgtcgccgccacccctccggccgtgacccgggcactgctgcagtaccggcacgaccagctgcccggcgcctaccacaacgcccagcagcaggggctcaaaggggccttgttcccgatggtgaccttcaacggcatcgagtgccacaatgaatgggaaatcaccttcgaggagctgcaccgtaacgcagcggtcgccttcgcgatttaccagtacacggcctacaccggcgatgaaagctacgtcaaccacgacggcatggaggtgctggtgggcatcagccgcttctgggcggaccgggtccacttctccaagcgcgccggcaagtacatgatccacggcgtcaccgggccgaacgagtacgaaaacaacgtcaacaacaactggtacaccaacacgatggccgcctggtgcctggagtacacgctggcccggctgccgaaggccgatgccgccattcaggccaagctggccgtgagcgccgccgagcagcgccagtggcaggacattatcgaccacatgtactatccggaggacaagaagctgggcatcttcgtccagcacgacaccttcctggataaggacctgcggccggcaagctcgattccggccgaccagcggccaatcaaccagcactggtcctgggaccgaatcctgcggtcgccgttcatcaagcaggcggatgtgctccagggcctgtacttcctgaacaatcgcttcacccgcgagcagaaggaacgcaattttgacttctacgagccgctgacggtgcacgaaagctcgctgagtgcctcgattcacgcggtgctggccgccgagctcggtaagcaggataaggccgttgaactctatcagcgtacggctcgtctggacctggacaactacaacaacgatacggcagacggtctgcacatcacctcgatgaccggcggctggctggctatcgtgcagggcttcgccggcatgcgctacgaccacgatcagctgcggttcgatccgttcctgccgaagcagtggcagggttaccagttccgcatcaactaccgcggccgggtgatccaggtcgcggtggggaaaaccgttgcagtgaccctgctggccggcccgccgctgaccgtcatggttgccggccagccgcagcatttggaggtgagcgcgcatgcttaaaggattgctgttcgacctcgacggcgtcttgaccgactcggccaagttccacctgcaggcctggagccagctggccacccagctgggcatcaccctgacgccggccgagcgcgaaggcctgcgcggccgctcgcggctggactcgctgaacctgattttggcggcaggcgcccaggaagaccggttcagtgccgcagagaaaacggcgctaaccgaccagaagaacgcggtgtacctgaagctgattcagacgatgacgccggtggacatcctgccgggcattccgcaactgctgaaggacgcgcaggcggccggcctgaaaatggcaatcgcctcggcgtcgcggaacgccccgacaattcttgaccacctgggcctggccgccagtttcgacgccatcgtcgatccggcgaccctgcaccgcggcaagcccgacccggagatctaccagcaggcgcaagcgctgctggggctccaggccgccgaggtgatcggcttcgaggatgcctcggccggggtcgccgccatcaaagcggccggtcagttcgcggttggcatcggggatgcccggcttctggccgcagcggattacctagtgaaagacacggcggccctgcagctgagccagttgcaagcggcgttcgccaaagaaagtggggagactaatggttgaaatcgacttggaccacctctacaagaagtacgacgacggcgaggattactcggtggtggacttcgaccttcacatcaaggataaggagttcatcgtgttcgtcggcccctcgggctgcgggaagtccaccacgctgcgtatgattgcggggctggaggacattaccaaaggcgagctgaaaatcgacgataaggtgatgaacgacgtggcccccaaggaccgcaacatcgccatggtgtttcagaactacgccttgtacccgcacatgtcagtgtacgacaacatggcgttcggcctaaagctacggcactacaagaaggaggacatcgacaaacgcgtgcaaaacgcggcggagatcctcggcctgaagccgtttctcgaccggaagccggccgccttgtccgggggccagcggcagcgggtggccttgggccgggccatcgtccgcgacgccccaattttcctgatggatgagccgttgtcgaacctggacgcgaagctgcgggtgtccatgcgggcggaaatcgccaagctccaccagcgcctgaacaccaccacgatttacgtgacccacgaccaaaccgaggccatgactatggccgaccgggttgtcgtcatgtccgttggccacgtgcagcagattggcaccccggccgagatttaccagaacccgcggaaccagttcgtggccgggttcatcgggtcgccggcgatgaacttcttcaacatgacctaccaggacggcttcgtcagcgacggccaaagcattcgcctcaaagtgccggaaggccgggcgaagattctggacgaccaagggtacaacggcaaggaagtcgtgttcggcatccggccggaggacatccattcggaggaggccttcctggagacctggccggacgcggttatcagctcaaccgtgtcggtgtcagagctcctgggcgccaccgagcagctttacctgaaggcggatgacaccgagtacgttgccaacgtcaacgcgcgcgacttccacaatcccggggatcatgtgaaaatgggcttcgacgtcaacaaggcgcacttcttcaacaaggacacgaccatggccatcgtggctaagccgattccgctggaaggctgaggaggtgagtgcatgaccccatggtggcagcaagccgtcatttaccagatctacccgaagagttttcaggacagcaacggggatggcatcggcgatttgccggggattaccagtcgccttgattaccttaagcggctgggcgtcgatgccctttggctgagcccagtgtatgtgtcgcccggcgaggacaacggctacgacatcgcggactacgaggccatcgatccccagttcgggacgatggccgacatggacgccttgatcgccgccgccaagcagcgcg

SEQ ID NO: 17 - DNA, 977 bp, Lacticaseibacillus paracasei
  source 1..977: mol_type = genomic DNA
cccgcgattttggcgtgattggcttcgacggggtattcctggaccaggtgtccaaccccaagctgaccacggtgaagcagcccgtgcagcgcctcggcgaactgctggcccgcatgctcctgcagaaggtggcacagtccggcgcccaacagggggagctgctggtcgatcctgagctgattgctcgggacacgacgcgaaagtagatcggatttcaactgtccttaccgctatggtagggccagtttttaggctctatgtcaaatctaattcatagctaatagttgatttggcaacgcctaaggcgtcagccatatcttggtaagtatgatggccttcactgaccagttgagctagcgcaccacgttgaaaacgtgataaagtagaagtacccaaagtaatcactccttatagctggttggaattaactactccattgtaagagattgctttgggcctttttttatttttgttcggattaattatagaatttgtctaattagttgaaaattcttagggttgcccatatatcttttagtctggtcattagtttttatgtttgatctgcttttttctgatcgcaaacacccacaactgcgagtgagtcctttttgaagtcgttgactgtcaacaatacatttatttccacattgacattgacagagccaaagcgcgttgccattagaactgcttccaaaaaagctaataactgtgagtcgcccaaacgtttgattagctaaatcgatacgcttttgcatactaatcctcccgcttgataagaaggtacttaaatagttgctttcaattgatctaatcgccattggcaccatgaaataaaggctaattcgtcaatctttggaatgccataggttctagcatacgttaacttttgagtggtgagtagttgatcatagggtttactaataataccaaccacaaggatatcgacttttttgtcaatcccgttgacaggcttt

SEQ ID NO: 18 - DNA, 1239 bp, Lacticaseibacillus paracasei
  source 1..1239: mol_type = genomic DNA
aaatacgcaaaagaacccgacgagagttaagtctcatcgggttctcagtcgtggatgaattagaagcattgttagctgcataaccttcaacataggatcaatagctggttagatggtcatctctcagactgtttgcaccagatccaggcaaacgtgtttatatccttggtcatactcaggatagatgggcattgtgagtgcaacaggacttagttgcttgtatgctaggcaatgttggccttgatacagaggatcactctgtttctgatctggatgataacctcgtttttccatttcaggacgctgtcccaataattgttgccctgggagtttgtcgaattaagtcttggtgccacaaacacacgctacggcttcttttcttgcaaactgatcttagtatttagggcagttgcatgattacggaattgaaccattttataatgaatcgtcttttctataagtctatagaaagtgcaggtaatggcattttctccagatcggattgtctaatcaatttaattgatttttttggtgtgtttgattatattgcttttgcaaaggtacaatataccttttctctgctgccttgcgagcagcgatggcatcctccatatgaacatatacacgatttaggacaagatggccttgaaagtacagccttgcgacccacttttgagcagttttatcccaactaactccgataacgccagatttgttattggaccgtttaagtgtagaagcaactaaatttgttcgattaattagttgaaaattcttgggattgcccatgtattttttagtctggttattagcttttatgtttgatctgcttatttctgatcgcaaacacccacaactgcgagtgagtcctttttgaagtcgttgactgtcaacgatacatttatttccacattgacattgacagagccaaagcgcgtttccattagaactgcttccaaaaaaactaatgactgtgagtcgtccaaacgtttgattggccaaatcgatacgcttttgcatactaatctccccgtttgataagaaggtacttaaagagttgttttcaattgatctagtcgccattggcaccatgaaataaaggctaattcgtcaatctttggaatgccataggttctagcatacgttaacttttgagtggtaagtagttgatcatagggtttgctaataataccaaccacaaggatatcgacatttttgtcaatcccgttgacaggtttt

SEQ ID NO: 19 - protein, 7 aa, synthetic construct
  REGION 1..7: note = peptide
PDLKDVG

SEQ ID NO: 20 - protein, 9 aa, synthetic construct
  REGION 1..9: note = peptide
LEIHLNAAQ

SEQ ID NO: 21 - protein, 10 aa, synthetic construct
  REGION 1..10: note = peptide
GTNFATIENA

SEQ ID NO: 22 - protein, 7 aa, synthetic construct
  REGION 1..7: note = peptide
GTDTRAE

SEQ ID NO: 23 - protein, 7 aa, synthetic construct
  REGION 1..7: note = peptide
GTDTRAE

SEQ ID NO: 24 - protein, 5 aa, synthetic construct
  REGION 1..5: note = peptide
YINAM

SEQ ID NO: 25 - protein, 4 aa, synthetic construct
  REGION 1..4: note = peptide
FMWQ

SEQ ID NO: 26 - protein, 5 aa, synthetic construct
  REGION 1..5: note = peptide
HLIQT

SEQ ID NO: 27 - protein, 9 aa, synthetic construct
  REGION 1..9: note = peptide
TAFAALTEL

SEQ ID NO: 28 - protein, 6 aa, synthetic construct
  REGION 1..6: note = peptide
MEANML

SEQ ID NO: 29 - protein, 12 aa, synthetic construct
  REGION 1..12: note = peptide
NDYEKIAMGLLS

SEQ ID NO: 30 - protein, 8 aa, synthetic construct
  REGION 1..8: note = peptide
FIPDLKDV

SEQ ID NO: 31 - protein, 10 aa, synthetic construct
  REGION 1..10: note = peptide
  REGION 6..10: note = misc_feature - Xaa can be any naturally occurring amino acid
MPTEQXXXXX

SEQ ID NO: 32 - protein, 7 aa, synthetic construct
  REGION 1..7: note = peptide
PDLKDVG

SEQ ID NO: 33 - protein, 7 aa, synthetic construct
  REGION 1..7: note = peptide
PDLKDVG

SEQ ID NO: 34 - protein, 12 aa, synthetic construct
  REGION 1..12: note = peptide
NDYEKIAMGLLS

SEQ ID NO: 35 - protein, 8 aa, synthetic construct
  REGION 1..8: note = peptide
FIPDLKDV

SEQ ID NO: 36 - protein, 10 aa, synthetic construct
  REGION 1..10: note = peptide
  REGION 6..10: note = misc_feature - Xaa can be any naturally occurring amino acid
MPTEQXXXXX

SEQ ID NO: 37 - protein, 7 aa, synthetic construct
  REGION 1..7: note = peptide
PDLKDVG

SEQ ID NO: 38 - protein, 12 aa, synthetic construct
  REGION 1..12: note = peptide
NDYEKIAMGLLS

SEQ ID NO: 39 - protein, 8 aa, synthetic construct
  REGION 1..8: note = peptide
FIPDLKDV

SEQ ID NO: 40 - protein, 10 aa, synthetic construct
  REGION 1..10: note = peptide
  REGION 6..10: note = misc_feature - Xaa can be any naturally occurring amino acid
MPTEQXXXXX

SEQ ID NO: 41 - protein, 8 aa, synthetic construct
  REGION 1..8: note = peptide
KLTEATVM